(19) United States
(12) Patent Application Publication
(10) Pub. No.: US 2023/0132823 A1
(43) Pub. Date: May 4, 2023

(54) BI-FUNCTIONAL COMPOUNDS AND METHODS FOR TARGETED UBIQUITINATION OF ANDROGEN RECEPTOR

(71) Applicant: MONTELINO THERAPEUTICS, INC., Southborough, MA (US)

(72) Inventors: Jenny DESANTIS, Foligno (IT); Roy Joseph VAZ, Southborough, MA (US); Michela ELEUTERI, Montefalco (IT)

(21) Appl. No.: 17/990,658

(22) Filed: Nov. 19, 2022

Related U.S. Application Data
(63) Continuation of application No. PCT/US2021/033039, filed on May 18, 2021.
(60) Provisional application No. 63/026,449, filed on May 18, 2020.
(60) Provisional application No. 63/050,735, filed on Jul. 10, 2020.
(60) Provisional application No. 63/183,371, filed on May 3, 2021.

(51) Int. Cl. C07D 417/14

(52) U.S. Cl. CPC C07D 417/14

(57) ABSTRACT

The present invention relates to bi-functional compounds which function to recruit endogenous proteins to an E3 ubiquitin ligase for degradation, and methods for using same. More specifically, the present disclosure provides specific proteolysis targeting chimera (PROTAC) molecules which find utility as modulators of targeted ubiquitination of a variety of polypeptides and other proteins, in particular the androgen receptor of a slice variant of AR which lacks the LBD, labelled as AR-V7, which are then degraded and/or otherwise inhibited by the compounds as described herein.

### CROSS-REFERENCE TO RELATED APPLICATIONS

This application is an international PCT Application which claims priority to and the benefit of U.S. Provisional Application Ser. No. 63/026,449, filed on May 18, 2020, U.S. Provisional Application Ser. No. 63/050,735, filed on Jul. 10, 2020, and U.S. Provisional Application Ser. No. 63/183,371, filed on May 3, 2021, the contents of each of which are hereby incorporated by reference in their entireties.

### FIELD OF THE INVENTION

This invention relates to therapeutic compounds and compositions, and methods for their use in the treatment of various indications, including various cancers. In particular, the invention relates to therapies and methods of treatment for cancers such as prostate cancer.

### BACKGROUND OF THE INVENTION

Prostate cancer is the most commonly diagnosed malignancy in males in the United States and the second leading cause of male cancer mortality. Numerous studies have shown that the androgen receptor (AR) is central not only to the development of prostate cancer, but also the progression of the disease to the castration resistance state (Taplin, M. E. et al., J. Clin. Oncol. 2003 21:2673-8; and Tilley, W. D. et al., Cancer Res. 1994 54:4096-4102). Thus, effective inhibition of human AR remains one of the most effective therapeutic approaches to the treatment of advanced, metastatic prostate cancer.

Androgens are also known to play a role in female cancers. One example is ovarian cancer where elevated levels of androgens are associated with an increased risk of developing ovarian cancer (Helzlsouer, K. J. et al., JAMA 1995 274, 1926-1930; Edmondson, R. J. et al., Br. J. Cancer 2002 86, 879-885). Moreover, AR has been detected in a majority of ovarian cancers (Risch, H. A., J. Natl. Cancer Inst. 1998 90, 1774-1786; Rao, B. R. et al., Endocr. Rev. 1991 12, 14-26; Clinton, G. M. et al., Crit. Rev. Oncol. Hematol. 1997 25, 1-9).

AR belongs to the nuclear hormone receptor family that is activated by androgens such as testosterone and dihydrotestosterone. These androgens, as well as antagonists such as enzalutamide, compete with the androgens that bind to the ligand binding domain (LBD). AR possesses a modular organization characteristic of all nuclear receptors. It is comprised of an N-terminal domain (NTD), a central DNA binding domain (DBD), a short hinge region, and C-terminal domain that contains a hormone ligand binding pocket (the LBD, which also comprises the hormone binding site (HBS)) and the Activation Function-2 (AF2) site (Gao, W. Q. et al., Chem. Rev. 2005 105:3352-3370). The latter represents a hydrophobic groove on the AR surface which is flanked with regions of positive and negative charges—“charge clamps” that are significant for binding AR activation factors (Zhou, X. E. et al., J. Biol. Chem. 2010 285:9161-9171).

The activation of AR follows a well characterized pathway: in the cytoplasm, the receptor is associated with chaperone proteins that maintain agonist binding conformation of the AR (Georget, V. et al., Biochemistry 2002 41:11824-11831). Upon binding of an androgen, the AR undergoes a series of conformational changes, disassociation from chaperones, dimerization, and translocation into the nucleus (Fang, Y. F. et al., J. Biol. Chem. 1996 271:28697-28702; and Wong, C. I. et al., J. Biol. Chem. 1993 268:19004-19012) where it further interacts with co-activator proteins at the AF2 site (Zhou, X. E. et al. J. Biol. Chem. 2010 285:9161-9171). This event triggers the recruitment of RNA polymerase II and other factors to form a functional transcriptional complex with the AR.

In castration-resistant prostate cancer (CRPC), drug resistance can manifest through AR-LBD mutations that convert AR-antagonists into agonists or by expression of AR-variants lacking the LBD. AR is a major driver of prostate cancer and inhibition of its transcriptional activity using competitive antagonists such as enzalutamide and apalutamide remains a frontline therapy for prostate cancer management. Another therapy is abiraterone which is an inhibitor of cytochrome P450 17A1 that impairs AR signaling by depleting adrenal and intratumoral testosterone and dihydrotestosterone. Recent work (Antonarakis, E. S. et al., New Engl. J. Med. 2014 37, 1028-1038) has shown that patients on enzalutamide and abiraterone with a splice variant of AR, labelled as AR-V7, had lower PSA response rates, shorter PSA progression-free survival, and shorter overall survival.

AR-V7 lacks the LBD, which is the target of enzalutamide and testosterone, but AR-V7 remains constitutively active as a transcription factor. Accordingly, it is desirable to investigate other approaches to antagonize the AR receptor as well as AR-V7. The common domain between these two proteins is the DBD and compounds have been identified as discussed in Li, H. et al., J. Med. Chem. 2014 57, 6458-6467 (2014); Dalal, K. et al., Mol. Cancer Ther. 2017 vol. 16, 2281-2291; Xu, R. et al., Chem. Biol. & Drug Design 2018 91(1), 172-180; and WO 2015/120543.

Several methods are available for the manipulation of protein levels, including bi-functional proteolysis targeting chimeric molecules (PROTACs) which contain a ligand that recognizes the target protein that is linked to a ligand that binds to a specific E3 ubiquitin ligase. The ensuing bifunctional molecule binds to the target protein and the E3 ligase enabling the transfer of ubiquitin to the target protein from the Ligase provided there is a suitable acceptor on the target protein. Another method is the “molecular glue” process whereby the molecule together with the E3 ligase recruit the target protein to the E3 ligase followed by the ubiquitin transfer and degradation of the target (Chopra, R., Sadok, A., Collins, I., Drug Disc Today: Technologies, 2019, 31, 5-13.) In the case of a compound acting as a “molecular glue”, the only requirement is the presence of an E3 ligase binding moiety. After binding to the E3 ligase, the ensuing moiety could recruit the protein to be degraded. The labelling of proteins with ubiquitin is implicated in the protein's turnover by the 26S proteasome.

Protein ubiquitination is a multi-step process whereby a ubiquitin protein is successively relayed between different classes of enzymes (E1, E2, E3) in order to eventually tag a cellular substrate. Initially, the C-terminal carboxylate of ubiquitin is adenylated by the E1 activating enzyme in an ATP-dependent step. Subsequently, a conserved nucleophilic cysteine residue of the E1 enzyme displaces the AMP from the ubiquitin adenylate resulting in a covalent ubiquitin thioester conjugate. The binding and ensuing adenylation of a second ubiquitin molecule promote the recruitment of an E2 conjugating enzyme to this ternary complex. An active site Cys on the E2 subsequently facilitates the transfer of the covalently linked ubiquitin from the E1 to a Cys residue on the E2 through a trans-thioesterification reaction. Concomitantly, an E3 ligase recruits a specific downstream target protein and mediates the transfer of the ubiquitin from the E2 enzyme to the terminal substrate through either a covalent or non-covalent mechanism. Each ubiquitin is ligated to a protein through either a peptide bond with the N-terminal amino group or an isopeptide bond formed between a side chain F-amino group of a select Lys residue on the target protein and the ubiquitin.

Deubiquitinating enzymes (DUBs) are enzymes that specifically cleave the ubiquitin protein from the substrate thereby offering additional mechanisms of regulation over the entire labeling pathway. In the current human proteome, there are eight known human E1s, about 40 E2s, over 600 E3s and over 100 DUBs. These enzymes are well described in Pavia, S. et al., J. Med. Chem. 2018 61(2), 405-421.

The E3 ligases originate in three major classes—the RING finger and U-box E3s, the HECT E3s, and the RING/HECT-hybrid type E3s. The E3 ligases are localized in various cell organelles and hence the effectiveness of the E3 ligase ligand depends at least in part on the location of the protein targeted for degradation, assuming that the full molecule is available within the appropriate location in the cell. In addition, for every combination of the target ligand and the ubiquitin recruiting ligand, the linker length and conformational flexibility also contributes to the effectiveness of the degradation molecule. The mechanism depends on the availability of a Lys residue on the surface of the protein close to the targeted protein ligand binding pocket. Ubiquitin binds at Lys residues and hence the “delivery” of ubiquitin for binding at the appropriate Lys influences the effectiveness of the degradation molecule. Crew et al. (US20170327469A1, US20180099940A1) are progressing a proposed treatment for castration-resistant prostate cancer based on bifunctional molecules coupling various E3 ligases to AR antagonists binding at the AR LBD site. Our approach is different in that we do not target the LBD site but the DBD site and, correspondingly, the chemical matter is quite different.

There exists a continuing need for effective treatments for diseases and conditions that are related to aberrant AR regulation or activity, for example, cancers such as prostate cancer, and Kennedy's Disease. In developing such treatments, it would be desirable to have a molecule which can simultaneously bind AR and an E3 ubiquitin ligase and which also promotes ubiquitination of AR-V7 and perhaps AR, and leads to degradation of AR-V7 and AR by the proteasome. Some compounds of this type have been described, for example, by the present Applicant in: US 2020/0239430, US 2020/0282068, and WO 2020/160295, the contents of each of which are hereby incorporated by reference in their entireties.

### SUMMARY OF THE INVENTION

The present disclosure relates to bi-functional compounds which function to recruit endogenous proteins to an E3 ubiquitin ligase for degradation, and methods for using same. More specifically, the present disclosure provides specific proteolysis targeting chimera (PROTAC) molecules which find utility as modulators of targeted ubiquitination of a variety of polypeptides and other proteins, such as AR, which are then degraded and/or otherwise inhibited by the compounds as described herein.

In one aspect, these PROTAC molecules comprise an E3 ubiquitin ligase binding moiety (i.e., a ligand for an E3 ubiquitin ligase) linked to a moiety that binds a target protein (i.e., a protein/polypeptide targeting ligand) such that the target protein/polypeptide is placed in proximity to the ubiquitin ligase to effect degradation (and/or inhibition) of that protein. In addition, the description provides methods for using an effective amount of the compounds described herein for the treatment or amelioration of a disease condition including cancer, e.g., prostate cancer, and Kennedy's Disease.

Suitable ligands that bind to the E3 ubiquitin ligase include cereblon binders such as immunomodulatory imide drugs (IMiDs) including thalidomide, pomalidomide, and lenalidomide (Deshales, R. J., Nature Chem Biol. 2015 11, 634-635), and analogs or derivatives thereof. The IMiDs themselves act as “molecular glues” and therefore have been shown to recruit a different set of proteins for degradation (reference). In addition, we have uncovered an intermediate molecule that acts via the “molecular glue” mechanism. Other suitable E3 ubiquitin ligase binders are E3 CRL2^{VHL} compounds, also called Von-Hippel-Lindau or VHL ligands, the cellular inhibitor of apoptosis protein (IAP) as discussed in Shibata, N. et al., J. Med. Chem., 2018 61(2), 543-575. Binders of the E3 ligase Mouse Double Minute 2 (MDM2) comprise the fourth class of E3 Ligase Binders (E3LBs) that are utilized (Skalniak, L., et al., Expert Opin. Ther, Patents, 2019, 29, 151-170).

In one aspect, the present disclosure provides compositions comprising such compounds which function to recruit proteins, including AR-V7 and AR, for targeted ubiquitination and degradation. In some embodiments, the structure of such compounds can be depicted as:
ARB-E3LB
   wherein ARB is an AR binding moiety and E3LB is a ubiquitin ligase binding moiety.

In some embodiments, the compounds may further comprise a chemical linker (“L”). The structure of such compounds can be depicted as:
ARB-L-E3LB
   wherein ARB is an AR binding moiety, L is a bond or linker moiety, and E3LB is a ubiquitin ligase binding moiety.

In an additional aspect, the present disclosure provides therapeutic compositions comprising an effective amount of a compound as described herein or pharmaceutically acceptable salt form thereof, and one or more pharmaceutically acceptable carriers. The therapeutic compositions modulate protein degradation in a patient or subject, for example, an animal such as a human, and can be used for treating or ameliorating disease states or conditions which are modulated through the degraded protein. In certain embodiments, the therapeutic compositions as described herein may be used to effectuate the degradation and/or inhibition of proteins of interest for the treatment or amelioration of a disease, e.g., cancer.

In another aspect, the present disclosure provides a method of ubiquitinating/degrading a target protein in a cell. In certain embodiments, the method comprises administering a bi-functional compound as described herein comprising an ARB moiety and a E3LB moiety, preferably linked through a linker moiety, as otherwise described herein, wherein the E3LB moiety is coupled to the ARB moiety and wherein the E3LB moiety recognizes an E3 ubiquitin ligase and the ARB moiety recognizes the target protein such that degradation of the target protein occurs when the target protein is placed in proximity to the ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cells of a patient.

In another aspect, the present disclosure provides methods for treating or ameliorating a disease, disorder or symptom thereof in a subject or a patient, e.g., an animal such as a human, comprising administering to a subject in need thereof a pharmaceutical composition comprising an effective amount, e.g., a therapeutically effective amount, of a compound as described herein or pharmaceutically acceptable salt form thereof, and a pharmaceutically acceptable carrier, wherein the composition is effective for treating or ameliorating the disease or disorder or symptom thereof in the subject.

In another aspect, the present disclosure provides methods for identifying the effects of the degradation of proteins of interest in a biological system using compounds according to the present disclosure.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the invention may be utilized in numerous combinations, all of which are expressly contemplated by the present description. These additional advantages objects and embodiments are expressly included within the scope of the present disclosure. The publications and other materials used herein to illuminate the background of the invention, and in particular cases, to provide additional details respecting the practice, are incorporated by reference. Where applicable or not specifically disclaimed, any one of the embodiments described herein are contemplated to be able to combine with any other one or more embodiments, even though the embodiments are described under different aspects of the disclosure.

### BRIEF DESCRIPTION OF THE DRAWINGS

FIG. 1 is an immunoblot of certain exemplified compounds.

### DETAILED DESCRIPTION

The following is a detailed description provided to aid those skilled in the art in practicing the invention of the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

The present disclosure relates to the surprising and unexpected discovery that an E3 ubiquitin ligase protein can ubiquitinate a target protein, in particular the androgen receptor of a slice variant of AR which lacks the LBD, labelled as AR-V7, once the E3 ubiquitin ligase protein and the target protein are brought into proximity by a chimeric construct (e.g., a PROTAC) as described herein, in which a moiety that binds the E3 ubiquitin ligase protein is coupled, e.g., covalently, to a moiety that binds the androgen receptor target protein. Accordingly, the present description provides compounds, compositions comprising the same, and associated methods of use for ubiquitination and degradation of a chosen target protein, e.g., androgen receptor AR-V7.

In one aspect, the present disclosure provides compounds useful for regulating protein activity. The composition comprises a ubiquitin pathway protein binding moiety (preferably for an E3 ubiquitin ligase, alone or in complex with an E2 ubiquitin conjugating enzyme which is responsible for the transfer of ubiquitin to targeted proteins) according to a defined chemical structure and a protein targeting moiety which are linked or coupled together, preferably through a linker, wherein the ubiquitin pathway protein binding moiety recognizes a ubiquitin pathway protein and the targeting moiety recognizes a target protein (e.g., androgen receptor). Such compounds may be referred to herein as PROTAC compounds or PROTACs.

In one aspect, the PROTACs of the present disclosure comprise an E3 ubiquitin ligase binding moiety (“E3LB”), and a moiety that binds a target protein (i.e., a protein/polypeptide targeting ligand) that is an AR binding moiety (“ARB”). In this embodiment, the structure of the bi-functional compound can be depicted as:
ARB-E3LB
   where ARB is an AR binding moiety as described herein, and E3LB is an E3 ligase binding moiety as described herein.

In certain embodiments the bi-functional compound further comprises a chemical linker (“L”). In these embodiments, the structure of the bi-functional compounds can be depicted as:
ARB-L-E3LB
   where ARB is an AR binding moiety as described herein, E3LB is an E3 ligase binding moiety as described herein, and L is a chemical linker moiety, e.g., a linker as described herein, or optionally a bond, that links the ARB and E3LB moieties.

The respective positions of the ARB and E3LB moieties as well as their number as illustrated herein is provided by way of example only and is not intended to limit the compounds in any way. As would be understood by the skilled artisan, the bi-functional compounds as described herein can be synthesized such that the number and position of the respective functional moieties can be varied as desired. In certain embodiments, the compounds as described herein comprise multiple E3LB moieties, multiple ARB moieties, multiple chemical linkers, or a combination thereof.

It will be understood that the general structures are exemplary and the respective moieties can be arranged spatially in any desired order or configuration, e.g., ARB-L-E3LB, and E3LB-L-ARB, respectively. The E3LB group and ARB group may be covalently linked to the linker group through any covalent bond which is appropriate and stable to the chemistry of the linker. It will be further understood that for all compounds described herein, one or more hydrogen atoms may be replaced with an equivalent number of deuterium atoms.

In certain embodiments, the ARB may be selected from the following structures:

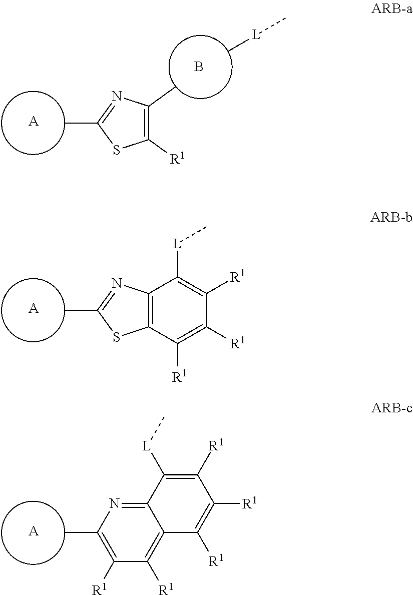

In each of ARB-a, ARB-b, and ARB-c, the following definitions apply:

L is the linker in the general formula above;

A is a 3-7 membered alicyclic with 0-4 heteroatoms, aryl, or heteroaryl, each optionally independently substituted by 1 or more halo, hydroxyl, nitro, CN, C≡CH, NR²R³, OCH₃, OC₁₋₃ alkyl (optionally substituted by 1 or more halo), CH₂F, CHF₂, CF₃, C₁₋₆ alkyl (linear, branched, optionally substituted by 1 or more halo, C₁₋₆ alkoxyl), C₁₋₆ alkoxyl (linear, branched, optionally substituted by 1 or more halo), C₂₋₆ alkenyl, C₂₋₆ alkynyl, or 3-6 membered alicyclic with 0-4 heteroatoms optionally substituted by 1 or more halo, hydroxyl, nitro, CN, C≡CH, CF₃, C₁₋₆ alkyl (linear, branched, optionally substituted by 1 or more halo, C₁₋₆ alkoxy), C₁₋₆ alkoxy (linear, branched, optionally substituted by 1 or more halo), C₂₋₆ alkenyl, or C₂₋₆ alkynyl;

B is aryl or heteroaryl, each optionally independently substituted by 1 or more halo, hydroxyl, nitro, CN, C≡CH, NR²R³, OCH₃, OC₁₋₃ alkyl (optionally substituted by 1 or more halo), CF₃, C₁₋₆ alkyl (linear, branched, optionally substituted by 1 or more halo, C₁₋₆ alkoxyl), C₁₋₆ alkoxyl (linear, branched, optionally substituted by 1 or more halo), C₂₋₆ alkenyl, C₂₋₆ alkynyl, or 3-6 membered alicyclic with 0-4 heteroatoms optionally substituted by 1 or more halo, hydroxyl, nitro, CN, C≡CH, CF₃, C₁₋₆ alkyl (linear, branched, optionally substituted by 1 or more halo, C₁₋₆ alkoxy), C₁₋₆ alkoxy (linear, branched, optionally substituted by 1 or more halo), C₂₋₆ alkenyl, or C₂₋₆ alkynyl; wherein the linker L is attached to B; and

each R¹ is independently H, OH, CONH₂, CONR²R³, SONH₂, SONR²R³, SO₂NH₂, SO₂NR²R³, NHCOC₁₋₃ alkyl (optionally substituted by 1 or more halo), NR²COC₁₋₃ alkyl (optionally substituted by 1 or more halo), NR²SO₂C₁₋₃ alkyl (optionally substituted by 1 or more halo), NR²SOC₁₋₃ alkyl (optionally substituted by 1 or more halo), CN, C≡CH, NH₂, NR²R³, OCH₃, OC₁₋₃ alkyl (optionally substituted by 1 or more halo), CHF₂, CH₂F, CF₃, halo, C₁₋₆ alkyl (linear, branched, optionally substituted by 1 or more halo, C₁₋₆ alkoxyl) or, if applicable, taken together with an R¹ on an adjacent bonded atom, together with the atoms they are attached to, form a 3-6 membered ring alicyclic, aryl, or heteroaryl system containing 0-2 heteroatoms; and

each R² and R³ is independently H, halo, C₁₋₆ alkyl (optionally substituted by 1 or more F) or taken together with the atom they are attached to, form a 3-8 membered ring system containing 0-2 heteroatoms.

In one aspect, A is:

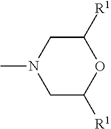

wherein R¹ is described above.

In another aspect, A is:

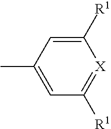

wherein R¹ is described above and X═C or N.

In yet another aspect, B is:

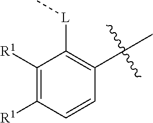

wherein L is the linker as described above, and R¹ is described above.

In still another aspect, B is:

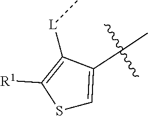

wherein L is the linker as described above, and R¹ is described above.

In yet another aspect, B is:

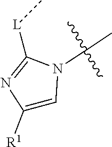

wherein L is the linker as described above, and R¹ is described above.

The linker group (L) comprises a chemical structural unit represented by the formula: -A_{q}-, in which q is an integer greater than 1; and A is independently selected from the group consisting of: a bond, CR^{L1}R^{L2}, O, S, SO, SO₂, NR^{L3} SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁ cycloalkyl optionally substituted with 0-6 R^{L1} and/or R^{L2} groups, and heteroaryl optionally substituted with 0-6 R^{L1} and/or R^{L2} groups; wherein R^{L1}, R^{L2}, R^{L3}, R^{L4} and R^{L5} are each independently selected from the group consisting of H, halo, C₁₋₈ alkyl, OC₁₋₈ alkyl, SC₁₋₈ alkyl, NHC₁₋₈ alkyl, N(C₁₋₈ alkyl)₂, C₃₋₁₁ cycloalkyl, aryl, heteroaryl, C₃₋₁₁ heterocyclyl, OC₁₋₈ cycloalkyl, SC₁₋₈ cycloalkyl, NHC₁₋₈ cycloalkyl, N(C₁₋₈cycloalkyl)₂, N(C₁₋₈ cycloalkyl)(C₁₋₈ alkyl), OH, NH₂, SH, SO₂C₁₋₈ alkyl, P(O)(OC₁₋₈ alkyl)(C₁₋₈ alkyl), P(O)(OC₁₋₈ alkyl)₂, CC—C₁₋₈ alkyl, CCH, CH═CH(C₁₋₈ alkyl), C(C₁₋₈ alkyl)═CH(C₁₋₈ alkyl), C(C₁₋₈ alkyl)═C(C₁₋₈ alkyl)₂, Si(OH)₃, SiC(₁₋₈ alkyl)₃, Si(OH)(C₁₋₈ alkyl)₂, COC₁₋₈ alkyl, CO₂H, CN, CF₃, CHF₂, CH₂F, NO₂, SF₅, SO₂NHC₁₋₈ alkyl, SO₂NHC₁₋₈ alkyl, SO₂N(C₁₋₈ alkyl)₂, SONHC₁₋₈ alkyl, SON(C₁₋₈ alkyl)₂, CONHC₁₋₈ alkyl, CON(C₁₋₈ alkyl)₂, N(C₁₋₈ alkyl)CONH(C₁₋₈ alkyl), N(C₁₋₈ alkyl)CON(C₁₋₈ alkyl)₂, NHCONH(C₁₋₈ alkyl), NHCON(C₁₋₈ alkyl)₂, NHCONH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl), NHSO₂N(C₁₋₈ alkyl)₂ and NHSO₂NH₂. In some embodiments, R^{L1} and R^{L2} each, independently can be linked to another A group to form a cycloalkyl and or heterocyclyl moiety that can be further substituted with 0-4 R^{L5} groups.

In certain embodiments, the E3LB moiety may be selected from a variety of moieties, including the following structures:

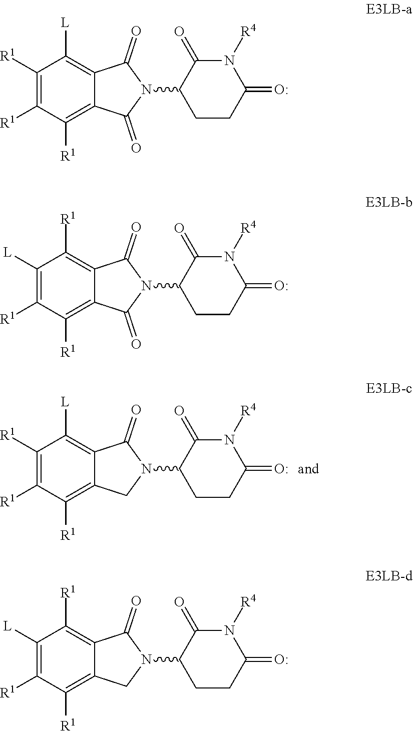

In each of E3LB-a through E3LB-d, the following definitions apply:

“ [symbol] ” in the above structures, represents a bond that may be stereospecific ((R) or (S)), or non-stereospecific;

R¹ is as described above;

R⁴ is selected from H, alkyl (linear, branched, optionally substituted with R⁵), OH, R⁵OCOOR⁶, R⁵OCONR⁵R⁷, CH₂-heterocyclyl optionally substituted with R⁵, or benzyl optionally substituted with R⁵;

R⁵ and R⁷ are each independently a bond, H, alkyl (linear, branched), cycloalkyl, aryl, hetaryl heterocyclyl, or —C(═O)R⁶ each of which is optionally substituted; and

R⁶ is selected from CONR⁵R⁷, OR⁵, NR⁵R⁷, SR⁵, SO₂R⁵, SO₂NR⁵R⁷, CR⁵R⁷, CR⁵NR⁵R⁷, aryl, hetaryl, alkyl (linear, branched, optionally substituted), cycloalkyl, heterocyclyl, P(O)(OR⁵)R⁷, P(O)R⁵R⁷, OP(O)(OR⁵)R⁷, OP(O)R⁵R⁷, Cl, F, Br, I, CF₃, CHF₂, CH₂F, CN, NR⁵SO₂NR⁵R⁷, NR⁵CONR⁵R⁷, CONR⁵COR⁷, NR⁵C(═N—CN)NR⁵R⁷, C(═N—CN)NR⁵R⁷, NR⁵C(—N═CN)R⁷, NR⁵C(═C—NO₂)NR⁵R⁷, SO₂NR⁵COR⁷, NO₂, CO₂R⁵, C(C═N—OR⁵)R⁷, CR⁵, CR⁵R⁷, CCR⁵, S(C═O)(C═N—R⁵)R⁷, SF₅, R⁵NR⁵R⁷, (R⁵O)ₙR⁷, or OCF₃, wherein n is an integer from 1 to 10.

The E3LB moiety may also be selected from E3LB-e and E3LB-f as described below:

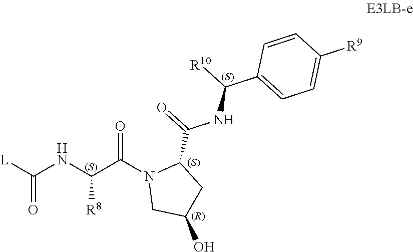

wherein L is the linker previously described; R⁸ is H, a straight chain or branched C₁₋₈ alkyl, C₃₋₆ cycloalkyl, halo, CFH₂, CF₂H, or CF₃; and R⁹ is a H, halo, 4-methylthiazol-5-yl, or oxazol-5-yl, and R¹⁰ is as defined below.

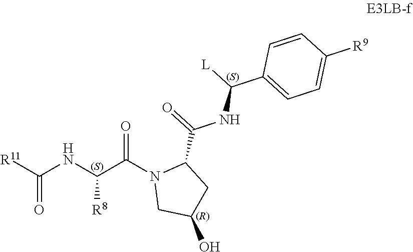

wherein L is the linker previously described and R¹¹ is independently optionally substituted alkyl, optionally substituted cycloalkyl, optionally substituted aryl, optionally substituted arylalkyl, optionally substituted arylalkoxy, optionally substituted heteroaryl, optionally substituted heterocyclyl, optionally substituted heterocycloalkyl wherein the substituents are alkyl, halogen, or OH.

The E3LB moiety may also be selected from E3LB-g, E3LB-h, E3LB-i, E3LB-j, and E3LB-k as described below:

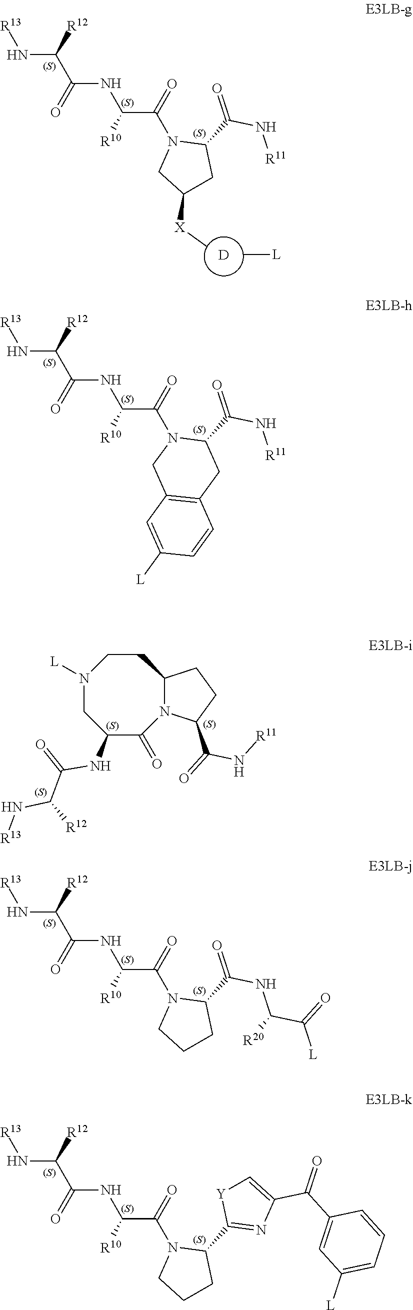

In each of E3LB-g, E3LB-h, E3LB-i, E3LB-j, and E3LB-k, the following definitions apply:

L is the linker previously described; R¹¹ is as defined above;

R¹⁰ are independently optionally substituted alkyl, optionally substituted cycloalkyl, optionally substituted cycloalkylalkyl, optionally substituted arylalkyl, optionally substituted aryl, optionally substituted thioalkyl wherein the substituents attached to the S atom of the thioalkyl are optionally substituted alkyl, optionally substituted branched alkyl, optionally substituted heterocyclyl, (CH₂)ᵥCOR¹⁴, CH₂CHR¹⁵COR¹⁶ or CH₂R¹⁷, where v=1 to 3; R¹⁴ and R¹⁶ are independently selected from OH, NR¹⁸R¹⁹, or —OR²⁰; R¹⁵ is —NR¹⁸R¹⁹; R¹⁷ is optionally substituted aryl or optionally substituted heterocyclyl, where the optional substituents include alkyl and halogen; R¹⁸ is hydrogen or optionally substituted alkyl; R¹⁹ is hydrogen, optionally substituted alkyl, optionally substituted branched alkyl, optionally substituted arylalkyl, optionally substituted heterocyclyl, —CH₂(OCH₂CH₂O)_{w}CH₃, or a polyamine chain, where w=1 to 8; and optional substituents may be OH, halo, or NH₂;

R¹² and R¹³ are independently hydrogen, optionally substituted alkyl, or optionally substituted cycloalkyl;

X is CH₂, N, or O; Y is S or O;

D is a bond (direct bond between X and L) or a ring which may be aryl or heteroaryl, said ring optionally substituted by 1 or more halo, hydroxyl, nitro, CN, C≡CH, NR²R³, OCH₃, OC₁₋₃ alkyl (optionally substituted by 1 or more halo), CF₃, C₁₋₆ alkyl (linear, branched, optionally substituted by 1 or more halo, C₁₋₆ alkoxyl), C₁₋₆ alkoxyl (linear, branched, optionally substituted by 1 or more halo), C₂₋₆ alkenyl, C₂₋₆ alkynyl, or 3-6 membered alicyclic with 0-4 heteroatoms optionally substituted by 1 or more halo, hydroxyl, nitro, CN, C≡CH, CF₃, C₁₋₆ alkyl (linear, branched, optionally substituted by 1 or more halo, C₁₋₆ alkoxy), C₁₋₆ alkoxy (linear, branched, optionally substituted by 1 or more halo), C₂₋₆ alkenyl, or C₂₋₆ alkynyl; R², R³ are independently H, halo, C₁₋₆ alkyl (optionally substituted by 1 or more F) or taken together with the atom they are attached to, form a 3-8 membered ring system containing 0-2 heteroatoms; and

R²⁰ is selected from the group consisting of:

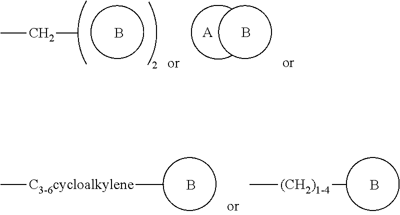

wherein A is a C₄₋₈ aliphatic ring, and B is an aryl or N-containing heteroaryl and optionally substituted by alkyl or haloalkyl.

Optionally, E3LB may be selected from the MDM2 class of E3 ligases represented by E3LB-1 below.

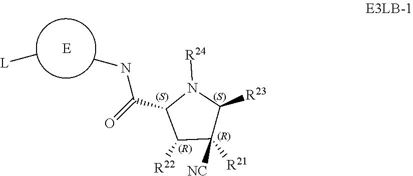

wherein R²² is independently aryl or heteroaryl optionally substituted by halogen, e.g., mono-, di or tri-substituted by halogen;

R²¹ is independently aryl or heteroaryl, optionally substituted (e.g., mono-, di- or tri-substituted) by halogen, CN, ethynyl, cyclopropyl, C₁₋₆ alkyl (e.g., methyl, ethyl, isopropyl), methoxy, ethoxy, isopropoxy, C₁₋₆ alkenyl, or C₁₋₆ alkynyl;

R²³ is selected from alkyl, substituted alkyl, alkenyl, substituted alkenyl, substituted alkynyl, aryl, substituted aryl, heteroaryl, substituted heteroaryl, cycloalkyl, substituted cycloalkyl, alkenyl and substituted cycloalkenyl;

R²⁴ is selected from H, alkyl, aryl, substituted alkyl, cycloalkyl, aryl substituted cycloalkyl, and alkoxy substituted cycloalkyl; and

E is para-substituted aryl, or single or multiple N containing heteroaryl, each optionally substituted by —OCH₃, —OCH₂CH₃ and halogen; and L is the linker previously defined above.

In other embodiments, the E3LB moiety may be selected from the following MDM2-binding moieties, E3LB-m, E3LB-n, E3LB-o, E3LB-p, and E3LB-q, wherein in each of which L is the linker previously defined above:

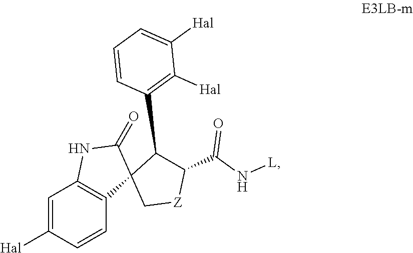

wherein Hal is a halogen (e.g., F, Cl, Br or I) and Z is CH₂, NH, N(C₁₋₆ alkyl), or O;

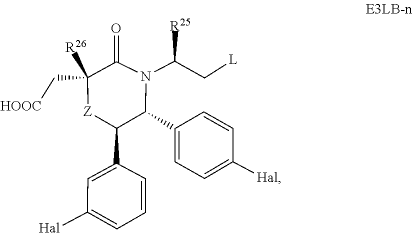

wherein Hal is a halogen (e.g., F, Cl, Br or I), R²⁵ is H or C₁₋₆ alkyl, R²⁶ is H or C₁₋₃ alkyl (e.g., methyl), and Z is CH₂, NH, N(C₁₋₆ alkyl), or O;

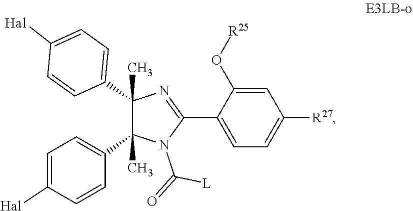

wherein Hal is a halogen (e.g., F, Cl, Br or I), R²⁵ is H or C₁₋₆ alkyl, and R²⁷ is H, C₁₋₃ alkyl, or C₁₋₃ alkoxy (e.g., methoxy);

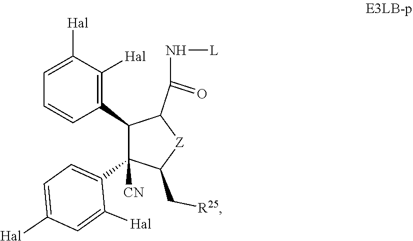

wherein Hal is a halogen (e.g., F, Cl, Br or I), R²⁵ is H or C₁₋₆ alkyl, and Z is CH₂, NH, N(C₁₋₆ alkyl), or O; and

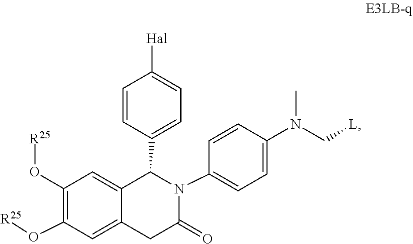

wherein Hal is a halogen (e.g., F, Cl, Br or I), and each R²⁵ is independently H or C₁₋₆ alkyl.

In another embodiment, the E3LB moiety is an cIAP moiety having the structure of E3LB-r, wherein L is the linker previously defined above:

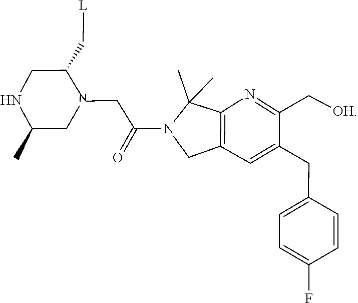

The E3LB moiety is inclusive of all cereblon binders such as immunomodulatory imide drugs (IMiDs) including thalidomide, pomalidomide, and lenalidomide, and analogs or derivatives thereof, as well as E3 CRL2^{VHL} compounds, the cellular inhibitor of apoptosis protein (IAP), and the mouse double minute 2 (MDM2) binders.

In certain embodiments, the compounds as described herein comprise a plurality of E3LB moieties and/or a plurality of ARB moieties. In certain additional embodiments, the compounds as described herein comprise multiple ARB moieties (targeting the same or different locations of the AR), multiple E3LB moieties, one or more moieties that bind specifically to another E3 ubiquitin ligase, e.g., VHL, IAP, MDM2, or a combination thereof. In any of the aspects of embodiments described herein, the ARB moieties, E3LB moieties, and other moieties that bind specifically to another E3 ubiquitin ligase can be coupled directly or via one or more chemical linkers or a combination thereof. In additional embodiments, where a compound has multiple moieties that bind specifically to another E3 ubiquitin ligase, the moieties can be for the same E3 ubiquitin ligase or each respective moiety can bind specifically to a different E3 ubiquitin ligase. In those embodiments where a compound has multiple ARB moieties, such moieties may be the same or, optionally, different.

In certain embodiments, where the compound comprises multiple E3LB moieties, the E3LB moieties are identical or, optionally, different. In additional embodiments, the compound comprising a plurality of E3LB moieties further comprises at least one ARB moiety coupled to a E3LB moiety directly or via a chemical linker (“L”) or both. In certain additional embodiments, the compound comprising a plurality of E3LB moieties further comprises multiple ARB moieties. In still additional embodiments, the ARB moieties are the same or, optionally, different.

In certain embodiments, the compound is selected from the group consisting of the exemplary compounds as described below, and salts and polymorphs thereof:

| | | |
|---|---|---|
| Example 1 | 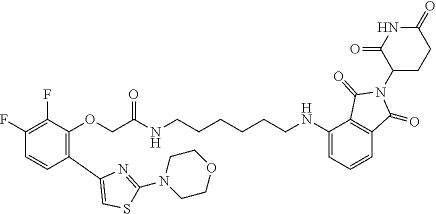 [SMILES: O=C(COc1c(-c2csc(N3CCOCC3)n2)ccc(F)c1F)NCCCCCCNc1cccc2c1C(=O)N(C1CCC(=O)NC1=O)C2=O] | 2-(2,3-difluoro-6-(2-morpholinothiazol-4- yl)phenoxy)-N-(6-((2-(2,6-dioxopiperidin-3-yl)- 1,3-dioxoisoindolin-4-yl)amino)hexyl)acetamide |
| Example 2 | 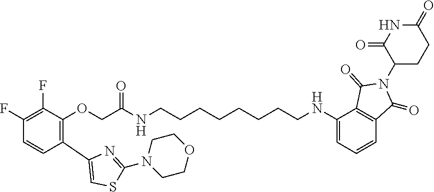 [SMILES: O=C(COc1c(-c2csc(N3CCOCC3)n2)ccc(F)c1F)NCCCCCCCCNc1cccc2c1C(=O)N(C1CCC(=O)NC1=O)C2=O] | 2-(2,3-difluoro-6-(2-morpholinothiazol-4- yl)phenoxy)-N-(8-((2-(2,6-dioxopiperidin-3-yl)- 1,3-dioxoisoindolin-4-yl)amino)octyl)acetamide |
| Example 3 | 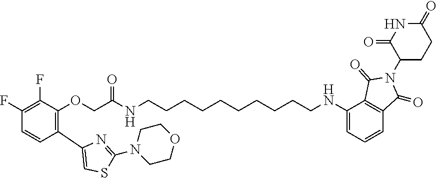 [SMILES: O=C(COc1c(-c2csc(N3CCOCC3)n2)ccc(F)c1F)NCCCCCCCCCCNc1cccc2c1C(=O)N(C1CCC(=O)NC1=O)C2=O] | 2-(2,3-difluoro-6-(2-morpholinothiazol-4- yl)phenoxy)-N-(10-((2-(2,6-dioxopiperidin-3-yl)- 1,3-dioxoisoindolin-4-yl)amino)decyl)acetamide |
| Example 4 | 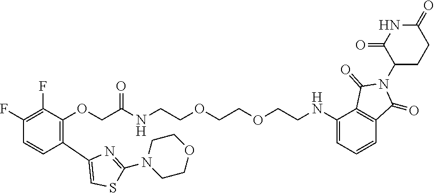 [SMILES: O=C(COc1c(-c2csc(N3CCOCC3)n2)ccc(F)c1F)NCCOCCOCCNc1cccc2c1C(=O)N(C1CCC(=O)NC1=O)C2=O] | 2-(2,3-difluoro-6-(2-morpholinothiazol-4- yl)phenoxy)-N-(2-(2-(2-((2-(2,6-dioxopiperidin-3- yl)-1,3-dioxoisoindolin-4- yl)amino)ethoxy)ethoxy)ethyl)acetamide |
| Example 5 | 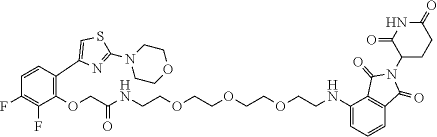 [SMILES: O=C(COc1c(-c2csc(N3CCOCC3)n2)ccc(F)c1F)NCCOCCOCCOCCNc1cccc2c1C(=O)N(C1CCC(=O)NC1=O)C2=O] | 2-(2,3-difluoro-6-(2-morpholinothiazol-4- yl)phenoxy)-N-(2-(2-(2-(2-((2-(2,6- dioxopiperidin-3-yl)-1,3-dioxoisoindolin-4- yl)amino)ethoxy)ethoxy)ethoxy)ethyl)acetamide |
| Example 6 | 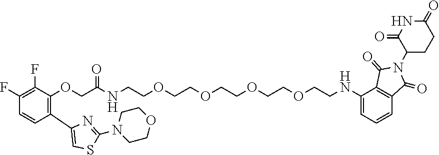 [SMILES: O=C(COc1c(-c2csc(N3CCOCC3)n2)ccc(F)c1F)NCCOCCOCCOCCOCCNc1cccc2c1C(=O)N(C1CCC(=O)NC1=O)C2=O] | 2-(2,3-difluoro-6-(2-morpholinothiazol-4- yl)phenoxy)-N-(14-((2-(2,6-dioxopiperidin-3-yl)- 1,3-dioxoisoindolin-4-yl)amino)-3,6,9,12- tetraoxatetradecyl)acetamide |
| Example 7 | 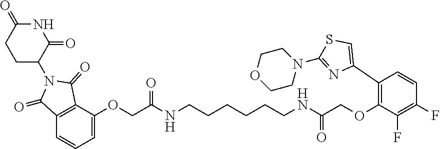 [SMILES: O=C(COc1cccc2c1C(=O)N(C1CCC(=O)NC1=O)C2=O)NCCCCCCNC(=O)COc1c(-c2csc(N3CCOCC3)n2)ccc(F)c1F] | 2-(2,3-difluoro-6-(2-morpholinothiazol-4- yl)phenoxy)-N-(6-(2-((2-(2,6-dioxopiperidin-3- yl)-1,3-dioxoisoindolin-4- yl)oxy)acetamido)hexyl)acetamide |
| Example 8 | 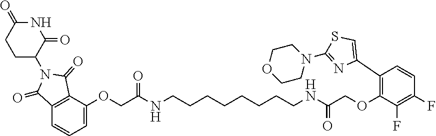 [SMILES: O=C(COc1cccc2c1C(=O)N(C1CCC(=O)NC1=O)C2=O)NCCCCCCCCNC(=O)COc1c(-c2csc(N3CCOCC3)n2)ccc(F)c1F] | 2-(2,3-difluoro-6-(2-morpholinothiazol-4- yl)phenoxy)-N-(8-(2-((2-(2,6-dioxopiperidin-3- yl)-1,3-dioxoisoindolin-4- yl)oxy)acetamido)octyl)acetamide |
| Example 9 | 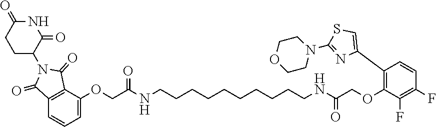 [SMILES: O=C(COc1cccc2c1C(=O)N(C1CCC(=O)NC1=O)C2=O)NCCCCCCCCCCNC(=O)COc1c(-c2csc(N3CCOCC3)n2)ccc(F)c1F] | 2-(2,3-difluoro-6-(2-morpholinothiazol-4- yl)phenoxy)-N-(10-(2-((2-(2,6-dioxopiperidin-3- yl)-1,3-dioxoisoindolin-4- yl)oxy)acetamido)decyl)acetamide |
| Example 10 | 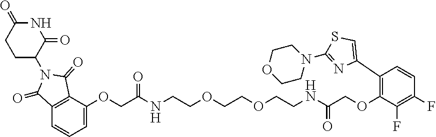 [SMILES: O=C(COc1cccc2c1C(=O)N(C1CCC(=O)NC1=O)C2=O)NCCOCCOCCNC(=O)COc1c(-c2csc(N3CCOCC3)n2)ccc(F)c1F] | 2-(2,3-difluoro-6-(2-morpholinothiazol-4- yl)phenoxy)-N-(2-(2-(2-(2-((2-(2,6- dioxopiperidin-3-yl)-1,3-dioxoisoindolin-4- yl)oxy)acetamido)ethoxy)ethoxy)ethyl)acetamide |
| Example 11 | 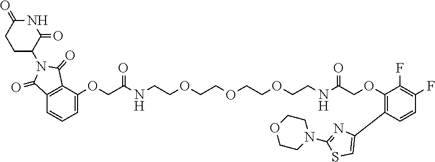 [SMILES: O=C(COc1cccc2c1C(=O)N(C1CCC(=O)NC1=O)C2=O)NCCOCCOCCOCCNC(=O)COc1c(-c2csc(N3CCOCC3)n2)ccc(F)c1F] | 2-(2,3-difluoro-6-(2-morpholinothiazol-4- yl)phenoxy)-N-(1-((2-(2,6-dioxopiperidin-3-yl)- 1,3-dioxoisoindolin-4-yl)oxy)-2-oxo-6,9,12- trioxa-3-azatetradecan-14-yl)acetamide |
| Example 12 | 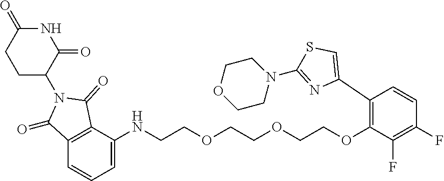 [SMILES: O=C1CCC(N2C(=O)c3cccc(NCCOCCOCCOc4c(-c5csc(N6CCOCC6)n5)ccc(F)c4F)c3C2=O)C(=O)N1] | 4-((2-(2-(2-(2,3-difluoro-6-(2-morpholinothiazol- 4-yl)phenoxy)ethoxy)ethoxy)ethyl)amino)-2-(2,6- dioxopiperidin-3-yl)isoindoline-1,3-dione |
| Example 13 | 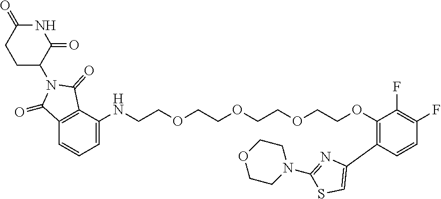 [SMILES: O=C1CCC(N2C(=O)c3cccc(NCCOCCOCCOCCOc4c(-c5csc(N6CCOCC6)n5)ccc(F)c4F)c3C2=O)C(=O)N1] | 4-((2-(2-(2-(2-(2,3-difluoro-6-(2- morpholinothiazol-4- yl)phenoxy)ethoxy)ethoxy)ethoxy)ethyl)amino)- 2-(2,6-dioxopiperidin-3-yl)isoindoline-1,3-dione |
| Example 14 | 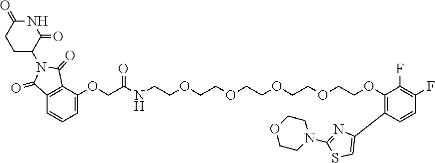 [SMILES: O=C(COc1cccc2c1C(=O)N(C1CCC(=O)NC1=O)C2=O)NCCOCCOCCOCCOCCOc1c(-c2csc(N3CCOCC3)n2)ccc(F)c1F] | N-(14-(2,3-difluoro-6-(2-morpholinothiazol-4- yl)phenoxy)-3,6,9,12-tetraoxatetradecyl)-2-((2- (2,6-dioxopiperidin-3-yl)-1,3-dioxoisoindolin-4- yl)oxy)acetamide |
| Example 15 | 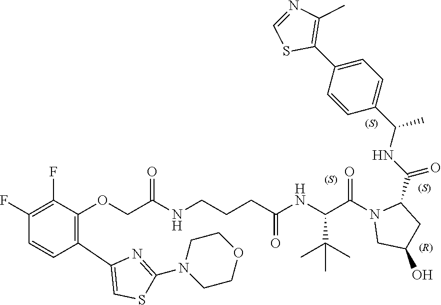 | (2S,4R)-1-((S)-2-(4-(2-(2,3-difluoro-6-(2- morpholinothiazol-4- yl)phenoxy)acetamido)butanamido)-3,3- dimethylbutanoyl)-4-hydroxy-N-((S)-1-(4-(4- methylthiazol-5-yl)phenyl)ethyl)pyrrolidine-2- carboxamide |
| Example 16 | 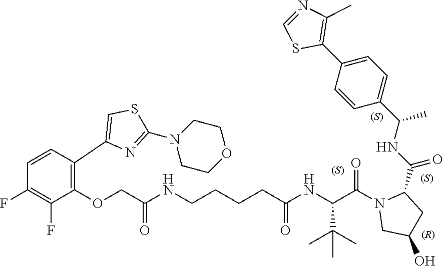 | (2S,4R)-1-((S)-2-(5-(2-(2,3-difluoro-6-(2- morpholinothiazol-4- yl)phenoxy)acetamido)pentanamido)-3,3- dimethylbutanoyl)-4-hydroxy-N-((S)-1-(4-(4- methylthiazol-5-yl)phenyl)ethyl)pyrrolidine-2- carboxamide |
| Example 17 | 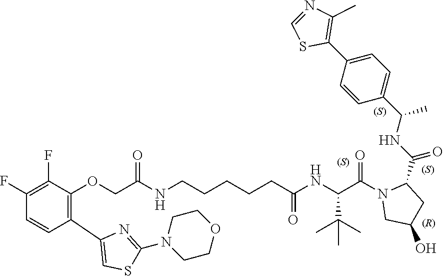 | (2S,4R)-1-((S)-2-(6-(2-(2,3-difluoro-6-(2- morpholinothiazol-4- yl)phenoxy)acetamido)hexanamido)-3,3- dimethylbutanoyl)-4-hydroxy-N-((S)-1-(4-(4- methylthiazol-5-yl)phenyl)ethyl)pyrrolidine-2- carboxamide |
| Example 18 | 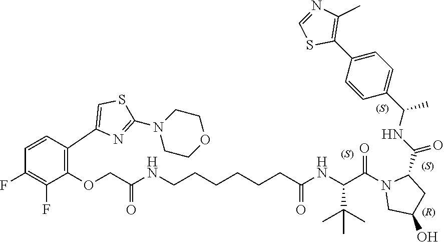 | (2S,4R)-1-((S)-2-(7-(2-(2,3-difluoro-6-(2- morpholinothiazol-4- yl)phenoxy)acetamido)heptanamido)-3,3- dimethylbutanoyl)-4-hydroxy-N-((S)-1-(4-(4- methylthiazol-5-yl)phenyl)ethyl)pyrrolidine-2- carboxamide |
| Example 19 | 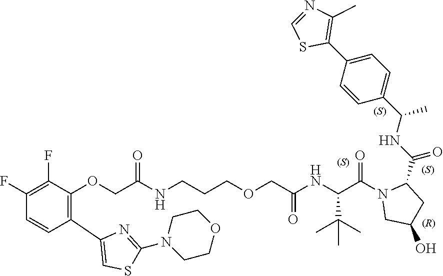 | (2S,4R)-1-((S)-2-(2-(3-(2-(2,3-difluoro-6-(2- morpholinothiazol-4- yl)phenoxy)acetamido)propoxy)acetamido)-3,3- dimethylbutanoyl)-4-hydroxy-N-((S)-1-(4-(4- methylthiazol-5-yl)phenyl)ethyl)pyrrolidine-2- carboxamide |
| Example 20 | 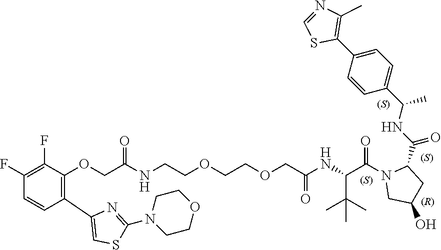 | (2S,4R)-1-((S)-2-(tert-butyl)-14-(2,3-difluoro-6- (2-morpholinothiazol-4-yl)phenoxy)-4,13-dioxo- 6,9-dioxa-3,12-diazatetradecanoyl)-4-hydroxy-N- ((S)-1-(4-(4-methylthiazol-5- yl)phenyl)ethyl)pyrrolidine-2-carboxamide |
| Example 21 | 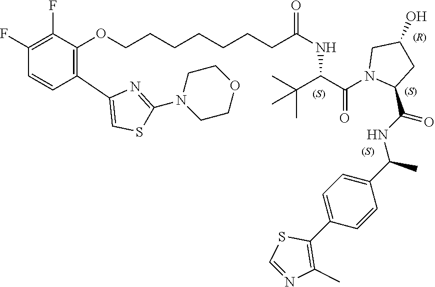 | (2S,4R)-1-((S)-2-(8-(2,3-difluoro-6-(2- morpholinothiazol-4-yl)phenoxy)octanamido)- 3,3-dimethylbutanoyl)-4-hydroxy-N-((S)-1-(4-(4- methylthiazol-5-yl)phenyl)ethyl)pyrrolidine-2- carboxamide |
| Example 22 | 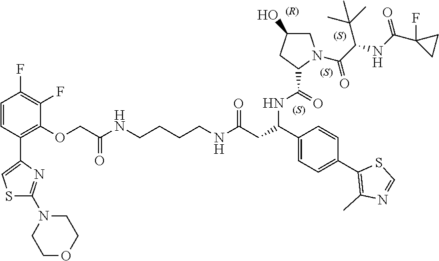 | (2S,4R)-N-((S)-3-((4-(2-(2,3-difluoro-6-(2- morpholinothiazol-4- yl)phenoxy)acetamido)butyl)amino)-1-(4-(4- methylthiazol-5-yl)phenyl)-3-oxopropyl)-1-((S)- 2-(1-fluorocyclopropane-1-carboxamido)-3,3- dimethylbutanoyl)-4-hydroxypyrrolidine-2- carboxamide |
| Example 23 | 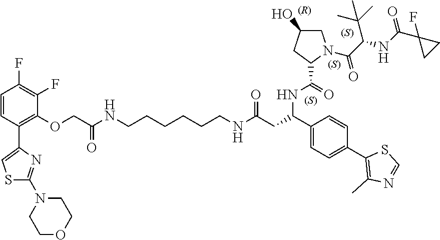 | (2S,4R)-N-((S)-3-((6-(2-(2,3-difluoro-6-(2- morpholinothiazol-4- yl)phenoxy)acetamido)hexyl)amino)-1-(4-(4- methylthiazol-5-yl)phenyl)-3-oxopropyl)-1-((S)- 2-(1-fluorocyclopropane-1-carboxamido)-3,3- dimethylbutanoyl)-4-hydroxypyrrolidine-2- carboxamide |
| Example 24 | 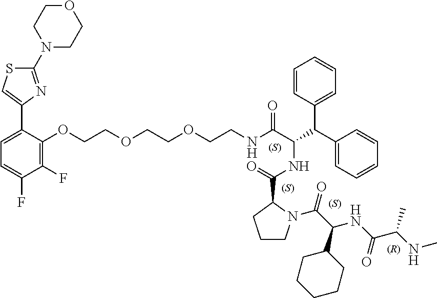 | (S)-1-((S)-2-cyclohexyl-2-((S)-2- (methylamino)propanamido)acctyl)-N-((S)-1-((2- (2-(2-(2,3-difluoro-6-(2-morpholinothiazol-4- yl)phenoxy)ethoxy)ethoxy)ethyl)amino)-1-oxo- 3,3-diphenylpropan-2-yl)pyrrolidine-2- carboxamide |
| Example 25 | 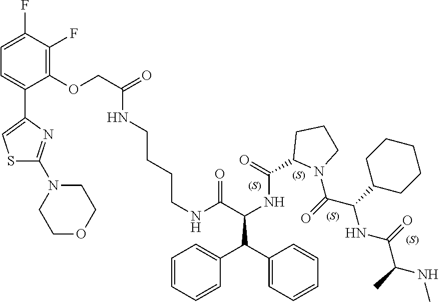 | (S)-1-((S)-2-cyclohexyl-2-((S)-2- (methylamino)propanamido)acetyl)-N-((S)-1-((4- (2-(2,3-difluoro-6-(2-morpholinothiazol-4- yl)phenoxy)acetamido)butyl)amino)-1-oxo-3,3- diphenylpropan-2-yl)pyrrolidine-2-carboxamide |
| Example 26 | 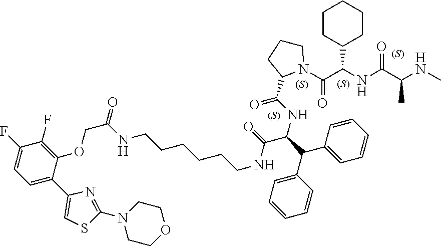 | (S)-1-((S)-2-cyclohexyl-2-((S)-2- (methylamino)propanamido)acctyl)-N-((S)-1-((6- (2-(2,3-difluoro-6-(2-morpholinothiazol-4- yl)phenoxy)acetamido)hexyl)amino)-1-oxo-3,3- diphenylpropan-2-yl)pyrrolidine-2-carboxamide |
| Example 27 | 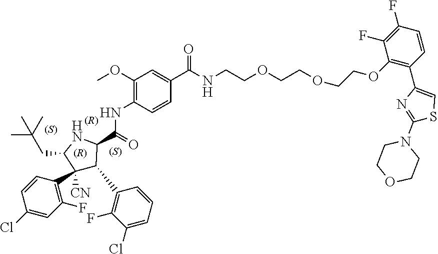 | (2R,3S,4R,5S)-3-(3-chloro-2-fluorophenyl)-4-(4- chloro-2-fluorophenyl)-4-cyano-N-(4-((2-(2-(2- (2,3-difluoro-6-(2-morpholinothiazol-4- yl)phenoxy)ethoxy)ethoxy)ethyl)carbamoyl)-2- methoxyphenyl)-5-neopentylpyrrolidine-2- carboxamide |
| Example 28 | 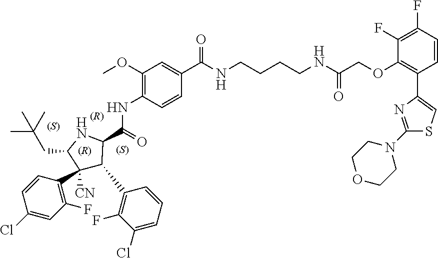 | (2R,3S,4R,5S)-3-(3-chloro-2-fluorophenyl)-4-(4- chloro-2-fluorophenyl)-4-cyano-N-(4-((4-(2-(2,3- difluoro-6-(2-morpholinothiazol-4- yl)phenoxy)acetamido)butyl)carbamoyl)-2- methoxyphenyl)-5-neopentylpyrrolidine-2- carboxamide |
| Example 29 | 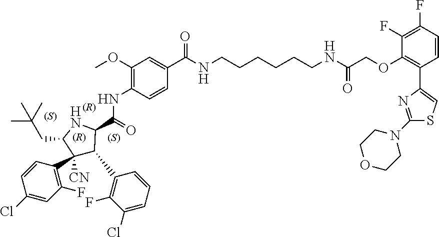 | (2R,3S,4R,5S)-3-(3-chloro-2-fluorophenyl)-4-(4- chloro-2-fluorophenyl)-4-cyano-N-(4-((6-(2-(2,3- difluoro-6-(2-morpholinothiazol-4- yl)phenoxy)acetamido)hexyl)carbamoyl)-2- methoxyphenyl)-5-neopentylpyrrolidine-2- carboxamide |
| Example 30 | 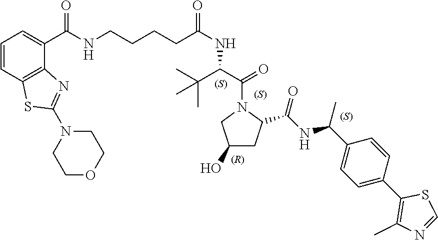 | N-(5-(((S)-1-((2S,4R)-4-hydroxy-2-(((S)-1-(4-(4- methylthiazol-5- yl)phenyl)ethyl)carbamoyl)pyrrolidin-1-yl)-3,3- dimethyl-1-oxobutan-2-yl)amino)-5-oxopentyl)-2- morpholinobenzo[d]thiazole-4-carboxamide |
| Example 31 | 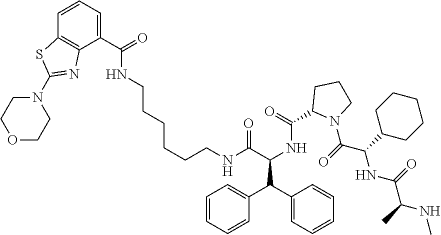 | N-(6-((S)-2-((S)-1-((S)-2-cyclohexyl-2-((S)-2- (methylamino)propanamido)acetyl)pyrrolidine-2- carboxamido)-3,3-diphenylpropanamido)hexyl)- 2-morpholinobenzo[d]thiazole-4-carboxamide |
| Example 32 | 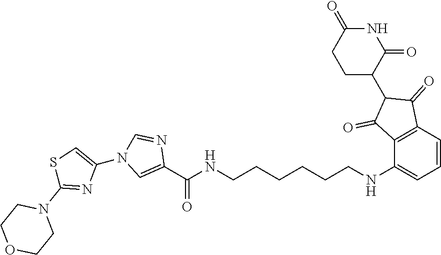 [SMILES: O=C1CCC(C2C(=O)c3cccc(NCCCCCCNC(=O)c4cn(-c5csc(N6CCOCC6)n5)cn4)c3C2=O)C(=O)N1] | N-(6-((2-(2,6-dioxopiperidin-3-yl)-1,3- dioxoisoindolin-4-yl)amino)hexyl)-1-(2- morpholinothiazol-4-yl)-1H-imidazole-4- carboxamide |
| Example 33 | 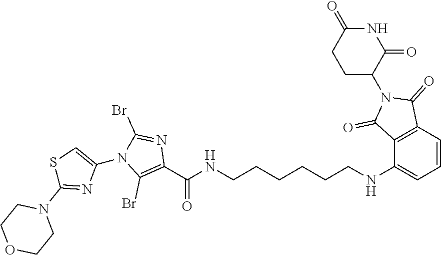 [SMILES: O=C1CCC(N2C(=O)c3cccc(NCCCCCCNC(=O)c4nc(Br)n(-c5csc(N6CCOCC6)n5)c4Br)c3C2=O)C(=O)N1] | 2,5-dibromo-N-(6-((2-(2,6-dioxopiperidin-3-yl)- 1,3-dioxoisoindolin-4-yl)amino)hexyl)-1-(2- morpholinothiazol-4-yl)-1H-imidazole-4- carboxamide |
| Example 34 | 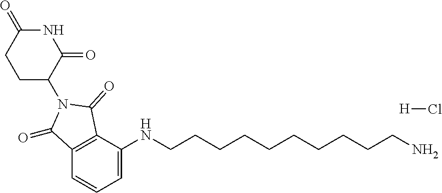 | 4-((10-aminodecyl)amino)-2-(2,6-dioxopiperidin- 3-yl)isoindoline-1,3-dione hydrochloride |
| Example 35 | 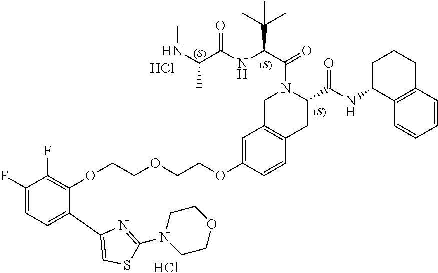 | (S)-7-(2-(2-(2,3-difluoro-6-(2-morpholinothiazol- 4-yl)phenoxy)ethoxy)ethoxy)-2-((S)-3,3- dimethyl-2-((S)-2- (methylamino)propanamido)butanoyl)-N-((R)- 1,2,3,4-tetrahydronaphthalen-1-yl)-1,2,3,4- tetrahydroisoquinoline-3-carboxamide dihydrochloride |
| Example 36 | 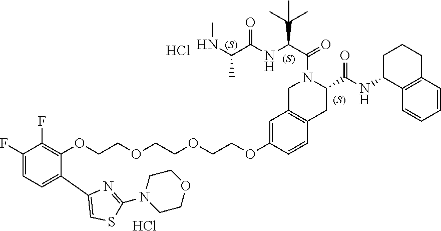 | (S)-7-(2-(2-(2-(2,3-difluoro-6-(2- morpholinothiazol-4- yl)phenoxy)ethoxy)ethoxy)ethoxy)-2-((S)-3,3- dimethyl-2-((S)-2- (methylamino)propanamido)butanoyl)-N-((R)- 1,2,3,4-tetrahydronaphthalen-1-yl)-1,2,3,4- tetrahydroisoquinoline-3-carboxamide dihydrochloride |
| Example 37 | 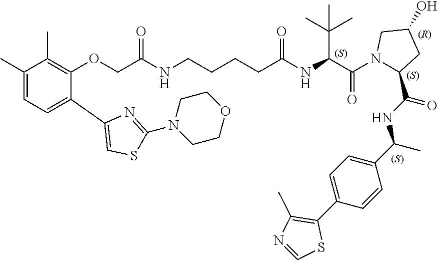 | (2S,4R)-1-((S)-2-(5-(2-(2,3-dimethyl-6-(2- morpholinothiazol-4- yl)phenoxy)acetamido)pentanamido)-3,3- dimethylbutanoyl)-4-hydroxy-N-((S)-1-(4-(4- methylthiazol-5-yl)phenyl)ethyl)pyrrolidine-2- carboxamide |
| Example 38 | 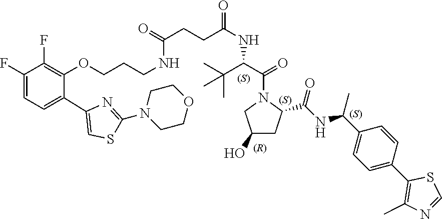 | N¹-(3-(2,3-difluoro-6-(2-morpholinothiazol-4- yl)phenoxy)propyl)-N⁴-((S)-1-((2S,4R)-4- hydroxy-2-(((S)-1-(4-(4-methylthiazol-5- yl)phenyl)ethyl)carbamoyl)pyrrolidin-1-yl)-3,3- dimethyl-1-oxobutan-2-yl)succinamide |
| Example 39 | 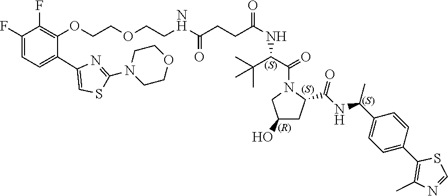 | N¹-(2-(2-(2,3-difluoro-6-(2-morpholinothiazol-4- yl)phenoxy)ethoxy)ethyl)-N⁴-((S)-1-((2S,4R)-4- hydroxy-2-(((S)-1-(4-(4-methylthiazol-5- yl)phenyl)ethyl)carbamoyl)pyrrolidin-1-yl)-3,3- dimethyl-1-oxobutan-2-yl)succinamide |
| Example 40 | 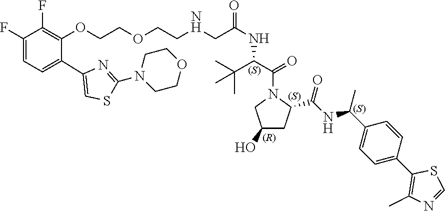 | (2S,4R)-1-((S)-2-(2-((2-(2-(2,3-difluoro-6-(2- morpholinothiazol-4- yl)phenoxy)ethoxy)ethyl)amino)acetamido)-3,3- dimethylbutanoyl)-4-hydroxy-N-((S)-1-(4-(4- methylthiazol-5-yl)phenyl)ethyl)pyrrolidine-2- carboxamide |
| Example 41 | 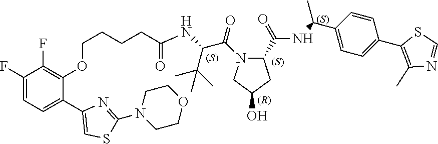 | (2S,4R)-1-((S)-2-(5-(2,3-difluoro-6-(2- morpholinothiazol-4-yl)phenoxy)pentanamido)- 3,3-dimethylbutanoyl)-4-hydroxy-N-((S)-1-(4-(4- methylthiazol-5-yl)phenyl)ethyl)pyrrolidine-2- carboxamide |
| Example 42 | 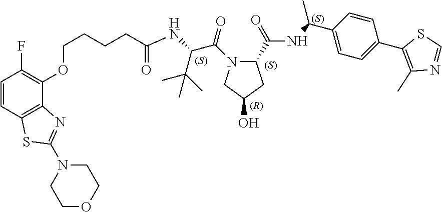 | (2S,4R)-1-((S)-2-(5-((5-fluoro-2- morpholinobenzo[d]thiazol-4- yl)oxy)pentanamido)-3,3-dimethylbutanoyl)-4- hydroxy-N-((S)-1-(4-(4-methylthiazol-5- yl)phenyl)ethyl)pyrrolidine-2-carboxamide |
| Example 43 | 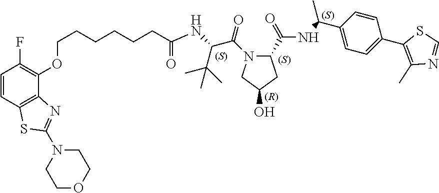 | (2S,4R)-1-((S)-2-(7-((5-fluoro-2- morpholinobenzo[d]thiazol-4- yl)oxy)heptanamido)-3,3-dimethylbutanoyl)-4- hydroxy-N-((S)-1-(4-(4-methylthiazol-5- yl)phenyl)ethyl)pyrrolidine-2-carboxamide |

In one aspect, the disclosure provides compounds of formula (I):

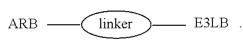

Such a compound is referred to as Androgen Receptor Binder-Linker-E3 Ligase Binder (I). It is understood that the terms “Androgen Receptor Binder,” “Androgen Receptor Binding Moiety” and “AR Binding Moiety” refer a molecular structure which generally binds successfully to androgen receptor protein, recognizing that in different people androgen receptors will not have the identical amino acid sequence, and thus, the strength of binding may vary across different particular AR sequences.

In further embodiments of this aspect, the present disclosure provides:
1.1 A compound having a chemical structure ARB-L-E3LB or ARB-Link-E3LB, wherein ARB is an AR binding moiety that does not bind to a ligand binding domain, E3LB is an E3 ligase binding moiety, and L or Link is a linker coupling the AR binding moiety to the E3 ligase binding moiety.
1.2 Compound 1.1, wherein the AR binding moiety binds to one more of AR splice variants V1 to V15, for example, to AR splice variant V7 (AR-V7).
1.3 Compound 1.1 or 1.2, wherein the AR binding moiety is selected from:

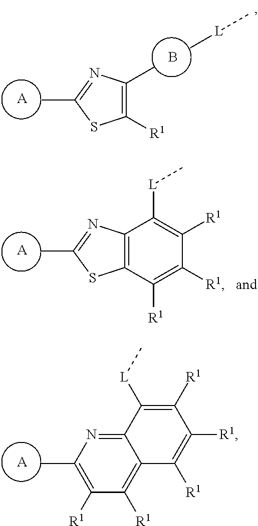

wherein.
   A is 3-7 membered alicyclic with 0-4 heteroatoms (e.g., morpholinyl) or aryl or heteroaryl, each optionally independently substituted by 1 or more halo, hydroxyl, nitro, CN, C≡CH, NR²R³, OCH₃, OC₁₋₃ alkyl (optionally substituted by 1 or more halo), CH₂F, CHF₂, CF₃, C₁₋₆ alkyl (linear, branched, optionally substituted by 1 or more halo, C₁₋₆ alkoxyl), C₁₋₆ alkoxyl (linear, branched, optionally substituted by 1 or more halo), C₂₋₆ alkenyl, C₂₋₆ alkynyl, or 3-6 membered alicyclic with 0-4 heteroatoms optionally substituted by 1 or more halo, hydroxyl, nitro, CN, C≡CH, CF₃, C₁₋₆ alkyl (linear, branched, optionally substituted by 1 or more halo, C₁₋₆ alkoxy), C₁₋₆ alkoxy (linear, branched, optionally substituted by 1 or more halo), C₂₋₆ alkenyl, or C₂₋₆ alkynyl;
   B is aryl (e.g., phenyl) or heteroaryl (e.g., imidazolyl), each optionally independently substituted by 1 or more halo, hydroxyl, nitro, CN, C≡CH, NR²R³, OCH₃, OC₁₋₃ alkyl (optionally substituted by 1 or more halo), CF₃, C₁₋₆ alkyl (linear, branched, optionally substituted by 1 or more halo, C₁₋₆ alkoxyl), C₁₋₆ alkoxyl (linear, branched, optionally substituted by 1 or more halo), C₂₋₆ alkenyl, C₂₋₆ alkynyl, or 3-6 membered alicyclic with 0-4 heteroatoms optionally substituted by 1 or more halo, hydroxyl, nitro, CN, CCH, CF₃, C₁₋₆ alkyl (linear, branched, optionally substituted by 1 or more halo, C₁₋₆ alkoxy), C₁₋₆ alkoxy (linear, branched, optionally substituted by 1 or more halo), C₂₋₆ alkenyl, or C₂₋₆ alkynyl; wherein the linker L is attached to B; and
   R¹ is each independently H, OH, CONH₂, CONR²R³, SONH₂, SONR²R³, SO₂NH₂, SO₂NR²R³, NHCO—C₁₋₃ alkyl (optionally substituted by 1 or more halo), NR²COC₁₋₃ alkyl (optionally substituted by 1 or more halo), NR²SO₂C₁₋₃ alkyl (optionally substituted by 1 or more halo), NR²SOC₁₋₃ alkyl (optionally substituted by 1 or more halo), CN, C≡CH, NH₂, NR²R³, OCH₃, OC₁₋₃ alkyl (optionally substituted by 1 or more halo), CHF₂, CH₂F, CF₃, halo, C₁₋₆ alkyl (linear, branched, optionally substituted by 1 or more halo, C₁₋₆ alkoxyl) or, if applicable, taken together with an R¹ on an adjacent bonded atom, together with the atoms they are attached to, form a 3-6 membered ring alicyclic, aryl, or heteroaryl system containing 0-2 heteroatoms; and
   R², R³ is independently H, halo, C₁₋₆ alkyl (optionally substituted by 1 or more F) or taken together with the atom they are attached to, form a 3-8 membered ring system containing 0-2 heteroatoms.
1.4 Compound 1.3, wherein A is:

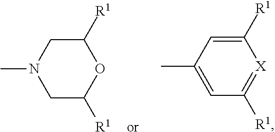

wherein X is CH or N.
1.5 Compound 1.4 or 1.5, wherein B is:

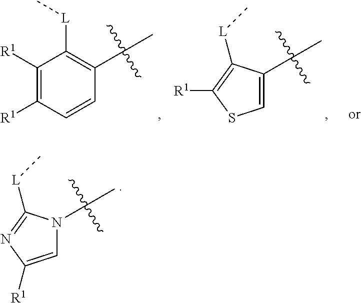

1.6 Compound 1.1 or 1.2, wherein the compound has an AR binding moiety as provided in a structure selected from the group consisting of:

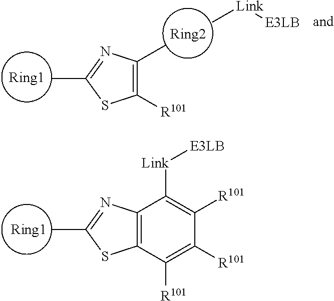

wherein:
   Ring 1 is a 3-7 membered alicyclic with 0-4 heteroatoms optionally substituted by 1 or more halo, CN, C≡CH, C₁₋₆ alkyl (linear, branched, optionally substituted by 1 or more halo, C₁₋₆ alkoxy), C₁₋₆ alkoxy (linear, branched, optionally substituted by 1 or more halo), C₂₋₆ alkenyl, or C₂₋₆ alkynyl, or a bridged or spiro bicyclic ring with 0-4 heteroatoms optionally substituted by 1 or more halo, CN, C≡CH, C₁₋₆ alkyl (linear, branched, optionally substituted by 1 or more halo, C₁₋₆ alkoxy), C₁₋₆ alkoxy (linear, branched, optionally substituted by 1 or more halo), C₂₋₆ alkenyl, C₂₋₆ alkynyl, or Ring 1 is

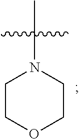 [SMILES: *N1CCOCC1]

Ring 2 is aryl (e.g., 2-benzyloxy-3,4-difluorophenyl) or heteroaryl optionally independently substituted by 1 or more halo, hydroxyl, CN, C≡CH, NR¹⁰²R¹⁰³, OCH3, OC₁₋₃ alkyl (optionally substituted by 1 or more halo), C₁₋₆ alkyl (linear branched, optionally substituted by 1 or more halo, C₁₋₆ alkoxyl), C₁₋₆ alkoxyl (linear, branched, optionally substituted by 1 or more halo), C₂₋₆ alkenyl, C₂₋₆ alkynyl, or 3-6 membered alicyclic with 0-4 heteroatoms optionally substituted with 1 or more halo, hydroxyl, CN, C≡CH, C₁₋₆ alkyl (linear, branched, optionally substituted by 1 or more halo, C₁₋₆ alkoxy), C₁₋₆ alkoxy (linear, branched, optionally substituted by 1 or more halo, C₁₋₆ alkoxy), C₁₋₆ alkoxyl (linear, branched, optionally substituted by 1 or more halo), C₂₋₆ alkenyl, or C₂₋₆ alkynyl; wherein R¹⁰² and R¹⁰³ are independently H, halo, C₁₋₆ alkyl (optionally substituted by 1 or more F) or, taken together with the atom they are attached to, form a 3-8 membered ring system containing 0-2 heteroatoms; or Ring 2 is

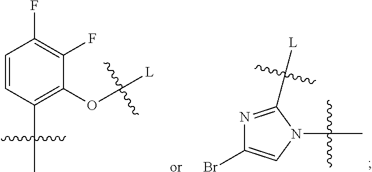

and
R¹⁰¹ is independently H, OH, CONH₂, CONR¹⁰²R¹⁰³, SONH₂, SONR¹⁰²R¹⁰³, SO₂NH₂, SO₂NR¹⁰²R¹⁰³, NHCO—C₁₋₃ alkyl (optionally substituted by 1 or more halo), NR¹⁰²COC₁₋₃ alkyl (optionally substituted by 1 or more halo), NR²SO₂C₁₋₃ alkyl (optionally substituted by 1 or more halo), NR¹⁰²SOC₁₋₃ alkyl (optionally substituted by 1 or more halo), CN, C≡CH, NH₂, NR¹⁰²R¹⁰³, OCH₃, OC₁₋₃ alkyl (optionally substituted by 1 or more halo), CHF₂, CH₂F, CF₃, halo, C₁₋₆ alkyl (linear, branched, optionally substituted by 1 or more halo, C₁₋₆ alkoxyl) or, taken together with an R¹⁰¹ on an adjacent bonded atom, together with the atoms they are attached to, form a 3-6 membered ring alicyclic, aryl, or heteroaryl system containing 0-2 heteroatoms, wherein R¹⁰² and R¹⁰³ are independently H, halo, C₁₋₆ alkyl (optionally substituted by 1 or more F) or, taken together with the atom they are attached to, form a 3-8 membered ring system containing 0-2 heteroatoms.
1.7 Any preceding compound, wherein the linker (“L” or “Link”) comprises a chemical structure represented by -A_{q}-, in which q is an integer greater than 1, and A is independently selected from the group consisting of: a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃11 cycloalkyl (optionally substituted with 0-6 R^{L1} and/or R^{L2} groups), and heteroaryl (optionally substituted with 0-6 R^{L1} and/or R^{L2} groups); wherein R^{L1}, R^{L2}, R^{L3}, R^{L4} and R^{L5} are each independently selected from the group consisting of H, halo, C₁₋₈ alkyl, OC₁₋₈ alkyl, SC₁₋₈ alkyl, NHC₁₋₈ alkyl, N(C₁₋₈ alkyl)₂, C₃₋₁₁ cycloalkyl, aryl, heteroaryl, C₃₋₁₁ heterocyclyl, OC₁₋₈ cycloalkyl, SC₁₋₈ cycloalkyl, NHC₁₋₈ cycloalkyl, N(C₁₋₈cycloalkyl)₂, N(C₁₋₈ cycloalkyl)(C₁₋₈ alkyl), OH, NH₂, SH, SO₂C₁₋₈ alkyl, P(O)(OC₁₋₈ alkyl)(C₁₋₈ alkyl), P(O)(OC₁₋₈ alkyl)₂, CC—C₁₋₈ alkyl, CCH, CH═CH(C₁₋₈ alkyl), C(C₁₋₈ alkyl)═CH(C₁₋₈ alkyl), C(C₁₋₈ alkyl)═C(C₁₋₈ alkyl)₂, Si(OH)₃, SiC(₁₋₈ alkyl)₃, Si(OH)(C₁₋₈ alkyl)₂, COC₁₋₈ alkyl, CO₂H, CN, CF₃, CHF₂, CH₂F, NO₂, SF₅, SO₂NHC₁₋₈ alkyl, SO₂NHC₁₋₈ alkyl, SO₂N(C₁₋₈ alkyl)₂, SONHC₁₋₈ alkyl, SON(C₁₋₈ alkyl)₂, CONHC₁₋₈ alkyl, CON(C₁₋₈ alkyl)₂, N(C₁₋₈ alkyl)CONH(C₁₋₈ alkyl), N(C₁₋₈ alkyl)CON(C₁₋₈ alkyl)₂, NHCONH(C₁₋₈ alkyl), NHCON(C₁₋₈ alkyl)₂, NHCONH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl), NHSO₂N(C₁₋₈ alkyl)₂ and NHSO₂NH₂; and wherein R^{L1} and R^{L2} each, independently may be linked to another A group to form a cycloalkyl and or heterocyclyl moiety that can be further substituted with 0-4 R^{L5} groups.
1.8 Any preceding compound, wherein the linker (e.g., “L” or “Link”) comprises a structure selected from the group consisting of:

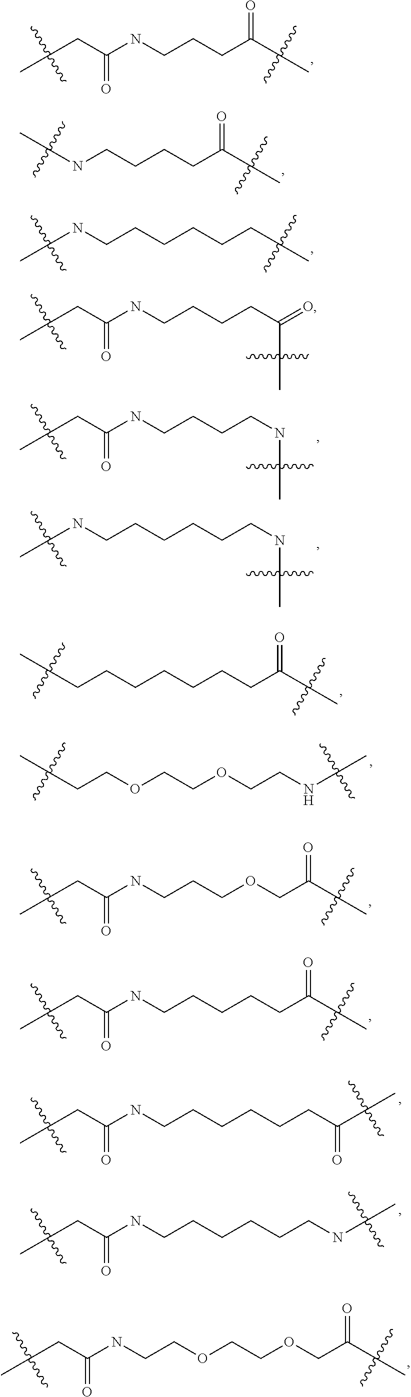

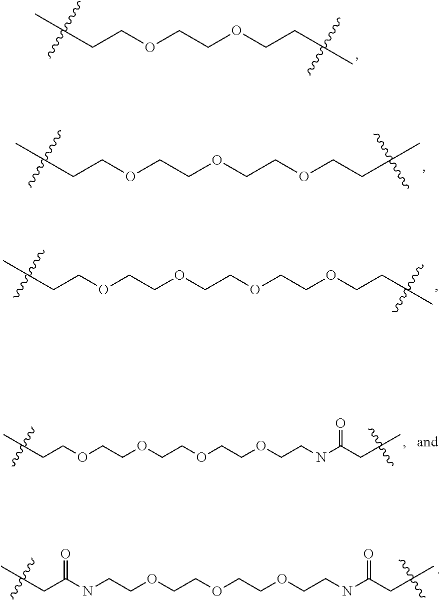 [SMILES: *CCOCCOCC* ; *CCOCCOCCOCC* ; *CCOCCOCCOCCOCC* ; *CCOCCOCCOCCOCCNC(=O)C* ; *CC(=O)NCCOCCOCCOCCNC(=O)C*]

1.9 Any preceding compound, wherein the E3 ligase binding moiety comprises a structure selected from the group consisting of:

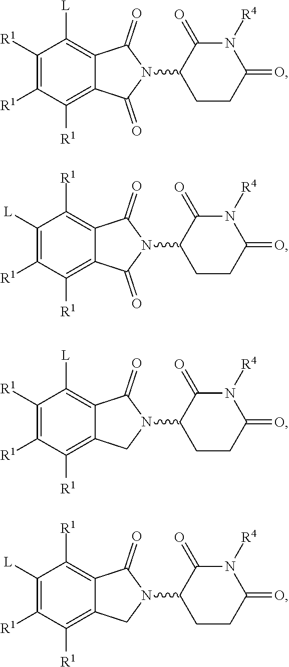

wherein “ [symbol] ” in the above structures, represents a bond that may be stereospecific ((R) or (S)), or non-stereospecific, and wherein:

R¹ is each independently H, OH, CONH₂, CONR²R³, SONH₂, SONR²R³, SO₂NH₂, SO₂NR²R³, NHCO—C₁₋₃ alkyl (optionally substituted by 1 or more halo), NR²COC₁₋₃ alkyl (optionally substituted by 1 or more halo), NR²SO₂C₁₋₃ alkyl (optionally substituted by 1 or more halo), NR²SOC₁₋₃ alkyl (optionally substituted by 1 or more halo), CN, C≡CH, NH₂, NR²R³, OCH₃, OC₁₋₃ alkyl (optionally substituted by 1 or more halo), CHF₂, CH₂F, CF₃, halo, C₁₋₆ alkyl (linear, branched, optionally substituted by 1 or more halo, C₁₋₆ alkoxyl) or, if applicable, taken together with an R¹ on an adjacent bonded atom, together with the atoms they are attached to, form a 3-6 membered ring alicyclic, aryl, or heteroaryl system containing 0-2 heteroatoms; and

R², R³ are each independently H, halo, C₁₋₆ alkyl (optionally substituted by 1 or more F) or taken together with the atom they are attached to, form a 3-8 membered ring system containing 0-2 heteroatoms;

R⁴ is selected from H, alkyl (linear, branched, optionally substituted with R⁵), OH, R⁵OCOOR⁶, R⁵OCONR⁵R⁷, CH₂-heterocyclyl optionally substituted with R⁵, or benzyl optionally substituted with R⁵;

R⁵ and R⁷ are each independently a bond, H, alkyl (linear, branched), cycloalkyl, aryl, hetaryl heterocyclyl, or —C(═O)R⁶ each of which is optionally substitute; and

R⁶ is selected from CONR⁵R⁷, OR⁵, NR⁵R⁷, SR⁵, SO₂R⁵, SO₂NR⁵R⁷, CR⁵R⁷, CR⁵NR⁵R⁷, aryl, hetaryl, alkyl (linear, branched, optionally substituted), cycloalkyl, heterocyclyl, P(O)(OR⁵)R⁷, P(O)R⁵R⁷, OP(O)(OR⁵)R⁷, OP(O)R⁵R⁷, Cl, F, Br, I, CF₃, CHF₂, CH₂F, CN, NR⁵SO₂NR⁵R⁷, NR⁵CONR⁵R⁷, CONR⁵COR⁷, NR⁵C(═N—CN)NR⁵R⁷, C(═N—CN)NR⁵R⁷, NR⁵C(—N═CN)R⁷, NR⁵C(═C—NO₂)NR⁵R⁷, SO₂NR⁵COR⁷, NO₂, CO₂R⁵, C(C═N—OR⁵)R⁷, CR⁵, CR⁵R⁷, CCR⁵, S(C═O)(C═N—R⁵)R⁷, SF₅, R⁵NR⁵R⁷, (R⁵O)ₙR⁷, or OCF₃, wherein n is an integer from 1 to 10.
1.10 Any preceding compound, wherein the E3 ligase binding moiety comprises a structure selected from:

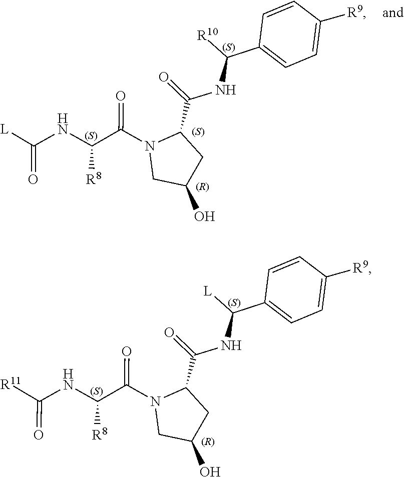

wherein in each moiety:
   R⁸ is H, a straight chain or branched C₁₋₈ alkyl (e.g., methyl, ethyl, isopropyl, tert-butyl), C₃₋₆ cycloalkyl (e.g., cyclopropyl), halo, CFH₂, CF₂H, or CF₃;
   R⁹ is a H, halo, 4-methylthiazol-5-yl, or oxazol-5-yl;
   R¹⁰ are each independently optionally substituted alkyl (e.g., methyl, ethyl, isopropyl, tert-butyl), optionally substituted cycloalkyl (e.g., cyclopropyl), optionally substituted cycloalkylalkyl, optionally substituted arylalkyl, optionally substituted aryl, optionally substituted thioalkyl wherein the substituents attached to the S atom of the thioalkyl are optionally substituted alkyl, optionally substituted branched alkyl, optionally substituted heterocyclyl, (CH₂)ᵥCOR¹⁴, CH₂CHR¹⁵COR¹⁶ or CH₂R¹⁷, where v=1 to 3;
   R¹⁴ and R¹⁶ are independently selected from OH, NR¹⁸R¹⁹, or —OR²⁰ (as defined hereinbelow);
   R¹⁵ is —NR¹⁸R¹⁹;
   R¹⁷ is optionally substituted aryl or optionally substituted heterocyclyl, wherein the optional substituents include alkyl and halogen;
   R¹⁸ is hydrogen or optionally substituted alkyl;
   R¹⁹ is hydrogen, optionally substituted alkyl, optionally substituted branched alkyl, optionally substituted arylalkyl, optionally substituted heterocyclyl, —CH₂(OCH₂CH₂O)_{w}CH₃, or a polyamine chain, where w=1 to 8;
   each R¹¹ is independently optionally substituted alkyl (e.g., methyl, ethyl, isopropyl, tert-butyl), optionally substituted cycloalkyl (e.g., cyclopropyl), optionally substituted aryl, optionally substituted arylalkyl, optionally substituted arylalkoxy, optionally substituted heteroaryl, optionally substituted heterocyclyl, optionally substituted heterocycloalkyl wherein the substituents are alkyl, halogen, or OH.
1.11 Any preceding compound, wherein the E3 ligase binding moiety comprises a structure selected from the group consisting of:

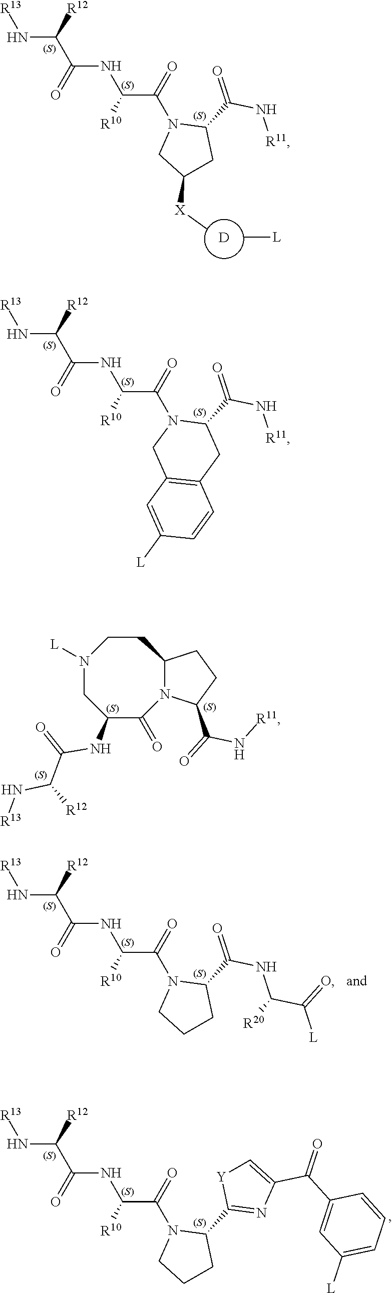

wherein:
   R¹⁰ and R¹¹ are each as defined in Compound 1.10;
   R¹² and R¹³ are independently hydrogen, optionally substituted alkyl (e.g., methyl), or optionally substituted cycloalkyl;
   X is CH₂, NR₂, or O;
   Y is S or O;
   D is a bond (direct bond between X and L) or a ring which may be aryl or heteroaryl, the ring optionally independently substituted by 1 or more halo, hydroxyl, nitro, CN, C≡CH, NR²R³, OCH₃, OC₁₋₃ alkyl (optionally substituted by 1 or more halo), CF₃, C₁₋₆ alkyl (linear, branched, optionally substituted by 1 or more halo, C₁₋₆ alkoxyl), C₁₋₆ alkoxyl (linear, branched, optionally substituted by 1 or more halo), C₂₋₆ alkenyl, C₂₋₆ alkynyl, or 3-6 membered alicyclic with 0-4 heteroatoms and substituted by 1 or more halo, hydroxyl, nitro, CN, C≡CH, CF₃, C₁₋₆ alkyl (linear, branched, optionally substituted by 1 or more halo, C₁₋₆ alkoxy), C₁₋₆ alkoxy (linear, branched, optionally substituted by 1 or more halo), C₂₋₆ alkenyl, or C₂₋₆ alkynyl;
   R², R³ are each independently H, halo, C₁₋₆ alkyl (optionally substituted by 1 or more F) or taken together with the atom they are attached to, form a 3-8 membered ring system containing 0-2 heteroatoms; and
   R²⁰ is selected from the group consisting of:

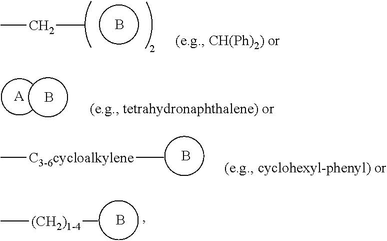

wherein A is a C₄₋₈ aliphatic ring, and B is an aryl (e.g., phenyl) or N-containing heteroaryl (e.g., pyridyl) and each is optionally substituted by alkyl or haloalkyl.
1.12 Any preceding compound, wherein the E3 ligase binding moiety is:

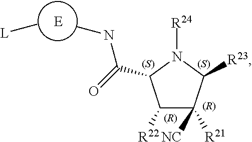

wherein:
   R²² is aryl (e.g., phenyl) or heteroaryl (e.g., pyridyl) optionally substituted by halogen (e.g., F or Cl), e.g., mono-, di or tri-substituted independently by halogen;
   R²¹ is aryl (e.g., phenyl) or heteroaryl (e.g., pyridyl), optionally substituted by halogen (e.g., F or Cl), e.g., mono-, di- or tri-substituted by halogen, CN, ethynyl, cyclopropyl, C₁₋₆ alkyl (e.g., methyl, ethyl, isopropyl), methoxy, ethoxy, isopropoxy, C₁₋₆ alkenyl and C₁₋₆ alkynyl;
   R²³ is selected from alkyl (e.g., methyl, ethyl, isopropyl, propyl, n-butyl, sec-butyl, isobutyl, t-butyl, n-pentyl, t-pentyl, isoamyl, neopentyl, n-hexyl), substituted alkyl, alkenyl, substituted alkenyl, substituted alkynyl, aryl, substituted aryl, heteroaryl, substituted heteroaryl, cycloalkyl, substituted cycloalkyl, alkenyl and substituted cycloalkenyl;
   R²⁴ is H, alkyl (e.g., methyl), aryl, substituted alkyl, cycloalkyl, aryl substituted cycloalkyl and alkoxy substituted cycloalkyl; and
   E is para-substituted (1,4-disubstituted) aryl (e.g., phenyl), or single or multiple N containing heteroaryl (e.g., pyridyl, pyrimidinyl, pyrazinyl, pyridazinyl), each optionally further substituted by —OCH₃, —OCH₂CH₃ and halogen.
1.13 Any preceding compound, wherein the E3 ligase binding moiety is a cereblon binding fragment, e.g., selected from thalidomide, pomalidomide, and lenalidomide.
1.14 Any preceding compound, wherein the E3 ligase binding moiety is an E3 CRL2^{VHL} moiety, or an IAP or MDM2-binding moiety.
1.15 Any preceding compound, wherein the compound has an E3 ligase binding moiety as provided in a structure selected from the group consisting of:

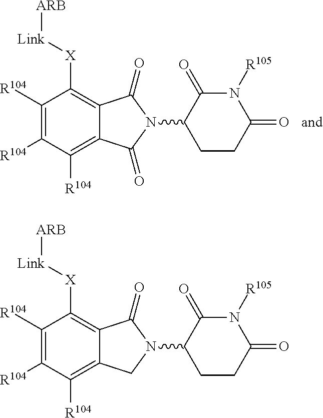

wherein:
   R¹⁰⁴ is independently H, OH, CONH₂, CONR¹⁰²R¹⁰³, SONH₂, SONR¹⁰²R¹⁰³, SO₂NH₂, SO₂NR¹⁰²R¹⁰³, NHCOC₁₋₃ alkyl (optionally substituted by one or more halo), NR¹⁰²COC₁₋₃ alkyl (optionally substituted by one or more halo), NR²SO₂C₁₋₃ alkyl (optionally substituted by one or more halo), NR¹⁰²SOC₁₋₃ alkyl (optionally substituted by one or more halo), CN, C≡CH, NH₂, NR¹⁰²R¹⁰³, OCH₃, OC₁₋₃ alkyl (optionally substituted by one or more halo), CHF₂, CH₂F, CF₃, halo, C₁₋₆ alkyl (linear, branched, optionally substituted by one or more halo, C₁₋₆ alkoxyl) or, taken together with an R¹⁰¹ on an adjacent bonded atom, together with the atoms they are attached to, form a 3-6 membered ring alicyclic, aryl, or heteroaryl system containing 0-2 heteroatoms; wherein and R¹⁰², R¹⁰³ is independently H, halo, C₁₋₆ alkyl (optionally substituted by one or more F) or taken together with the atom they are attached to, form a 3-8 membered ring system containing 0-2 heteroatoms;
   R¹⁰⁵ is independently H, C₁₋₆ alkyl (optionally substituted by one or more F); and
   X is NH or O.
1.16 Any preceding compound, wherein the compound has an E3 ligase binding moiety which is a Von-Hippel-Lindau Ligase binding moiety as provided in a structure selected from the group consisting of:

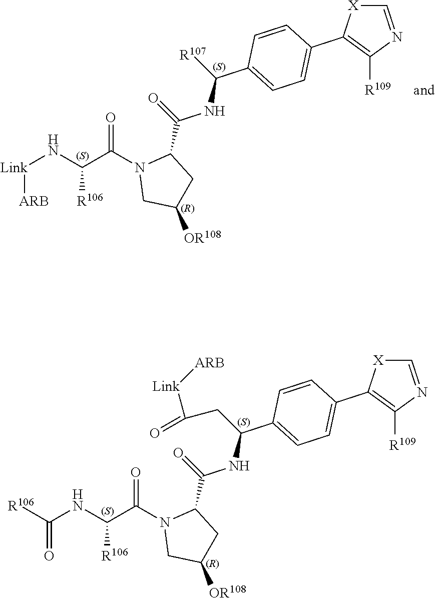

wherein
   R¹⁰⁶ is isopropyl, tert-butyl, sec-butyl, cyclopentyl, cyclohexyl, cyclopropyl or haloalkyl;
   R¹⁰⁷ is H, haloalkyl, methyl, ethyl, isopropyl, cyclopropyl or C₁-C₆ alkyl (linear, branched, optionally substituted), each optionally substituted with one or more halo, hydroxyl, CN, C₁-C₆ alkyl (linear, branched, optionally substituted), or C₁-C₆ alkoxyl (linear, branched, optionally substituted);
   R¹⁰⁸ is H or a prodrug group;
   R¹⁰⁹ is H, halo, optionally substituted C₃₋₆ cycloalkyl, optionally substituted C₁₋₆ alkyl, optionally substituted C₁₋₆ alkenyl or C₁₋₆ haloalkyl; and
   X is S or O.
1.17 Any preceding compound, wherein the compound has an E3 ligase binding moiety as provided in a structure selected from the group consisting of:

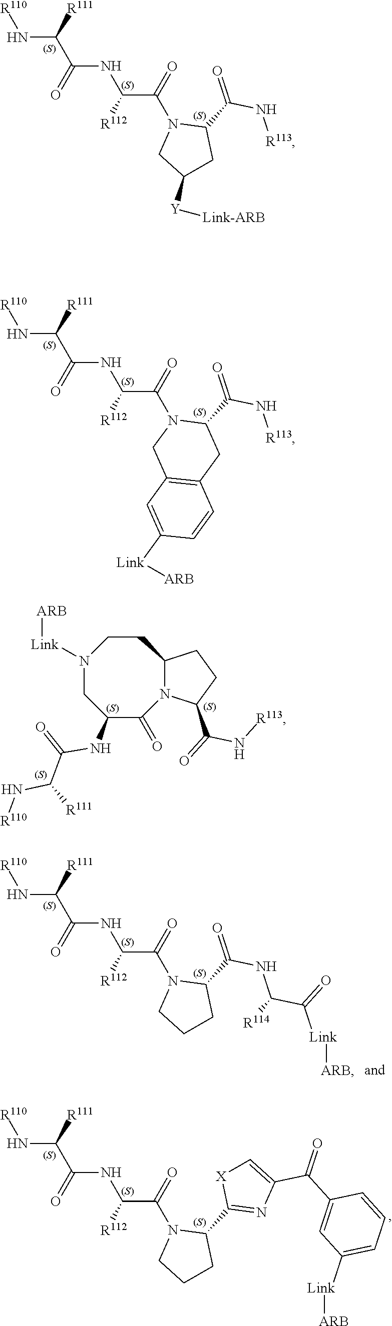

wherein:
R¹¹⁰ are independently hydrogen, optionally substituted alkyl or optionally substituted cycloalkyl;
R¹¹¹ are independently hydrogen, optionally substituted alkyl or optionally substituted cycloalkyl;
R¹¹² are independently optionally substituted alkyl, optionally substituted cycloalkyl, optionally substituted cycloalkylalkyl, optionally substituted arylalkyl, optionally substituted aryl, optionally substituted thioalkyl wherein the substituents attached to the S atom of the thioalkyl are optionally substituted branched alkyl, optionally substituted heterocyclyl, —(CH₂)ᵥCOR¹¹⁵, —CH₂CHR¹¹⁶COR¹¹⁷ or CH₂R¹¹⁸, where v=1-3;
R¹¹⁵ and R¹¹⁷ are independently selected from OH, NR¹¹⁸R¹¹⁹ or OR¹²⁰;
R¹¹⁶ is NR¹¹⁸R¹¹⁹;
R¹¹⁸ is optionally substituted aryl or optionally substituted heterocyclyl where the optional substituents include alkyl and halogen; and
R¹¹⁹ is hydrogen or optionally substituted alkyl; and
R¹¹³ is selected from the group consisting of

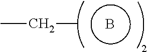

where B is an aryl or N-containing heteroaryl and optionally substituted by alkyl or haloalkyl;
R¹¹⁴ is selected from the group consisting of

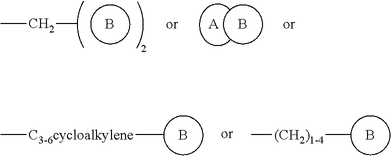

wherein A is a C₄₋₈ aliphatic ring, B is an aryl or N-containing heteroaryl and optionally substituted by alkyl or haloalkyl;
Y is N, O, C═O, or S, and
X is S or O.
1.18 Any preceding compound, wherein the compound has an E3 ligase binding moiety which is an MDM2 homolog inhibitor as provided in a structure selected from the group consisting of:

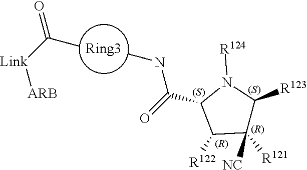

wherein:
Ring 3 is para-substituted aryl, or single or multiple N containing heteroaryl optionally substituted by —OCH3, —OCH2CH3, or halogen;
R¹²¹ is independently aryl or heteroaryl, optionally substituted (e.g., mono-, di- or tri-substituted) by halogen, —CN, ethynyl, cyclopropyl, C₁₋₆ alkyl (e.g., methyl, ethyl, isopropyl), methoxy, ethoxy, isopropoxy, C₁₋₆ alkenyl and C₁₋₆ alkynyl;
R¹²² is independently aryl or heteroaryl optionally substituted by halogen, or mono, di, or tri-substituted halogen;
R¹²³ is selected from alkyl, substituted alkyl, alkenyl, substituted alkenyl, substituted alkynyl, aryl, substituted aryl, heteroaryl, substituted heteroaryl, cycloalkyl, substituted cycloalkyl, alkenyl and substituted cycloalkenyl; and
R¹²⁴ is selected from H, alkyl, aryl, substituted alkyl, cycloalkyl, aryl substituted cycloalkyl and alkoxy substituted cycloalkyl.
1.19 Any preceding compound, wherein the AR binding moiety is selected from:

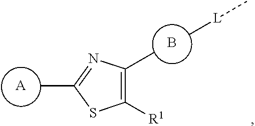

wherein:
   A is a 3-7 membered alicyclic ring with 0-4 heteroatoms (e.g., morpholinyl), B is aryl (e.g., phenyl) or heteroaryl (e.g., imidazolyl) optionally substituted by one or more halo, and R¹ is H, OH, CN, NH₂, OCH₃, halo, or C₁₋₆ alkyl; or

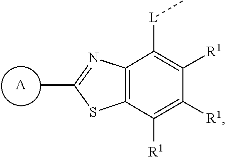

wherein:
A is a 3-7 membered alicyclic ring with 0-4 heteroatoms (e.g., morpholinyl), and R¹ is H, OH, CN, NH₂, OCH₃, halo, or C₁₋₆ alkyl.
1.20 Compound 1.19, wherein A is selected from morpholinyl, piperazinyl, N-methylpiperazinyl, piperidinyl, and pyrrolidinyl.
1.21 Compound 1.20, wherein A is morpholinyl (e.g., 1-morpholinyl).
1.22 Any of Compounds 1.19 to 1.21, wherein R¹ is H or halo.
1.23 Any of Compounds 1.19 to 1.22, wherein B is phenyl optionally substituted by one or two halo (e.g., fluoro, chloro or bromo) or B is imidazolyl optionally substituted by one or two halo (e.g., fluoro, chloro or bromo).
1.24 Compound 1.23, wherein B is:

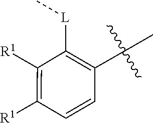

wherein each R¹ is independently H or F.
1.25 Any preceding compound, wherein the AR binding moiety is selected from:

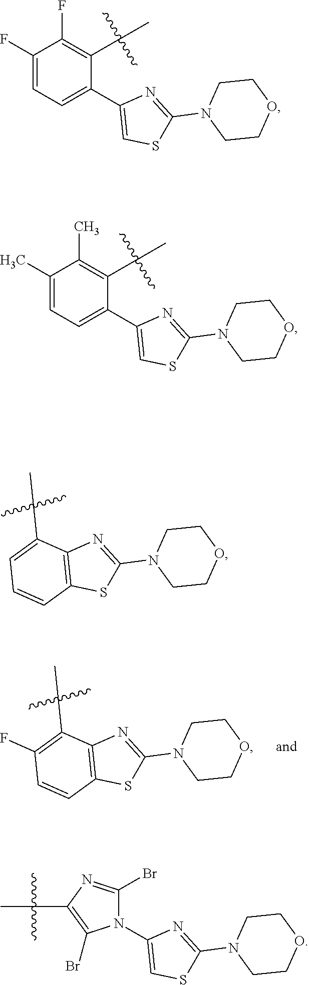 [SMILES: *c1c(-c2csc(N3CCOCC3)n2)ccc(F)c1F ; *c1c(-c2csc(N3CCOCC3)n2)ccc(C)c1C ; *c1cccc2sc(N3CCOCC3)nc12 ; *c1c(F)ccc2sc(N3CCOCC3)nc12 ; *c1nc(Br)n(-c2csc(N3CCOCC3)n2)c1Br]

1.26 Any preceding compound, wherein the AR binding moiety is:

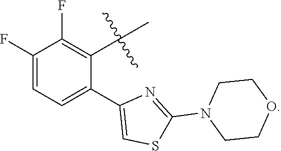 [SMILES: *c1c(-c2csc(N3CCOCC3)n2)ccc(F)c1F]

1.27 Any preceding compound, wherein the E3 ligase binding moiety is selected from:

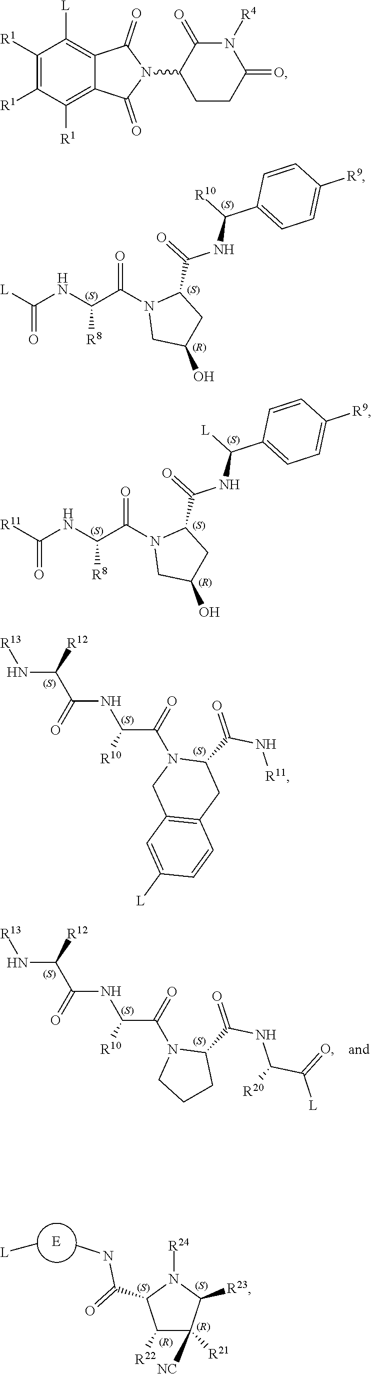

each as described hereinabove.
1.28 Compound 1.27, wherein each R¹ (of the E3LB) is independently selected from H, OH, CN, NH₂, OCH₃, halo, or C₁₋₆ alkyl; R⁴ is H or C₁₋₃ alkyl (e.g., methyl); R⁸ is H, halo, or C₁₋₆ alkyl (e.g., methyl, ethyl, isopropyl, tert-butyl); R¹⁰ is H, halo, or optionally substituted C₁₋₆ alkyl (e.g., methyl, ethyl, isopropyl, tert-butyl) or C₃₋₆ cycloalkyl (e.g., cyclopropyl); R¹¹ is H, optionally substituted C₁₋₆ alkyl (e.g., methyl, ethyl, isopropyl, tert-butyl), or optionally substituted C₃₋₁₀ cycloalkyl (e.g., cyclopropyl); R¹² and R¹³ are each independently H or C₁₋₆ alkyl (e.g., methyl); R²⁰ is CH(aryl)₂ (e.g. CHPh₂); R²¹ and R²² are each aryl (e.g., phenyl) optionally substituted by halogen (e.g., F or Cl); R²³ optionally substituted C₁₋₁₀ alkyl (e.g., methyl, ethyl, isopropyl, propyl, n-butyl, sec-butyl, isobutyl, t-butyl, n-pentyl, t-pentyl, isoamyl, neopentyl, n-hexyl); R²⁴ is H or C₁₋₆ alkyl; and E is para-substituted phenyl optionally substituted by OCH₃.
1.29 Compound 1.27, wherein R¹ is H; R⁴ is H; R⁸ is C₁₋₆ alkyl (e.g., tert-butyl); R¹⁰ is optionally substituted C₁₋₆ alkyl (e.g., methyl or tert-butyl) or C₃₋₆ cycloalkyl (e.g., cyclohexyl); R¹¹ is optionally substituted C₃₋₁₀ cycloalkyl (e.g., cyclopropyl or tetrahydronapthyl); R¹² and R¹³ are each independently C₁₋₆ alkyl (e.g. methyl); R²⁰ is CH(phenyl)₂; R²¹ and R²² are each phenyl optionally substituted by one or two halogen (e.g., F or Cl); R²³ C₁₋₆ alkyl (e.g., tert-amyl); R²⁴ is H; and E is para-substituted phenyl optionally substituted by OCH₃.
1.30 Compound 1.27, wherein R¹ is H; R⁴ is H; R⁸ is tert-butyl; R¹⁰ is methyl, tert-butyl or cyclohexyl; R¹¹ is cyclopropyl or tetrahydronapthyl each optionally substituted with halo (e.g., fluoro); R¹² and R¹³ are each independently methyl; R²⁰ is CH(phenyl)₂; R²¹ and R²² are each phenyl optionally substituted by one or two halogen (e.g., F or Cl); R²³ tert-amyl; R²⁴ is H; and E is para-substituted phenyl substituted by one OCH₃.
1.31 Any preceding compound, wherein the E3 ligase binding moiety is selected from the group consisting of:

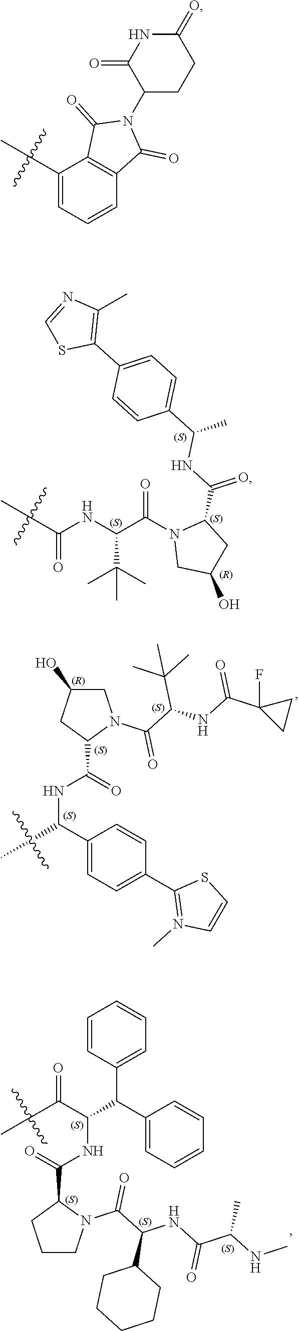

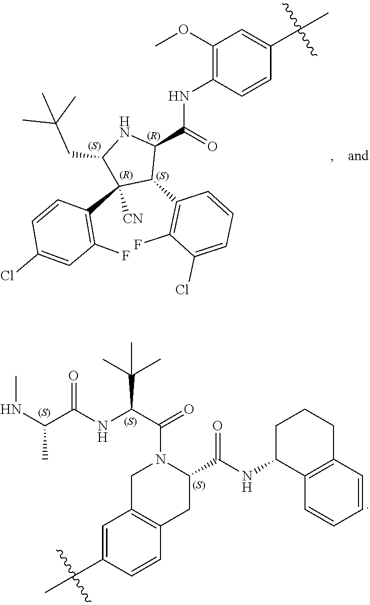

1.32 Any preceding compound, wherein the E3 ligase binding moiety is selected from the group consisting of:

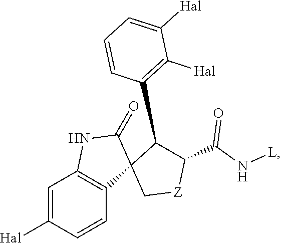

wherein Hal is a halogen (e.g., F, Cl, Br or I) and Z is CH₂, NH, N(C₁₋₆ alkyl), or O;

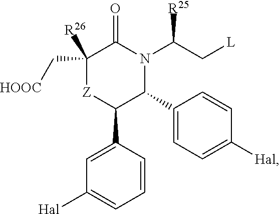

wherein Hal is a halogen (e.g., F, Cl, Br or I), R²⁵ is H or C₁₋₆ alkyl, R²⁶ is H or C₁₋₃ alkyl (e.g., methyl), and Z is CH₂, NH, N(C₁₋₆ alkyl), or O;

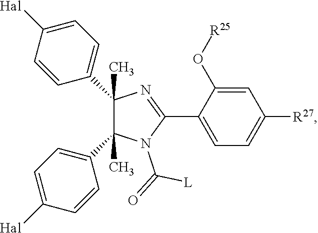

wherein Hal is a halogen (e.g., F, Cl, Br or I), R²⁵ is H or C₁₋₆ alkyl, and R²⁷ is H, C₁₋₃ alkyl, or C₁₋₃ alkoxy (e.g., methoxy);

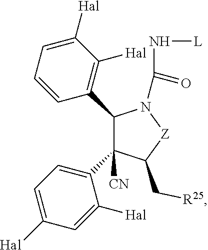

wherein Hal is a halogen (e.g., F, Cl, Br or I), R²⁵ is H or C₁₋₆ alkyl, and Z is CH₂, NH, N(C₁₋₆ alkyl), or O; and

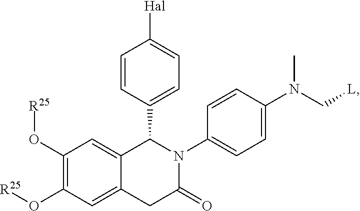

wherein Hal is a halogen (e.g., F, Cl, Br or I), and each R²⁵ is independently H or C₁₋₆ alkyl.
1.33 Any preceding compound, wherein the E3 ligase binding moiety is:

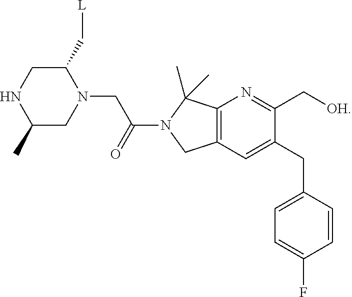

1.34 Any preceding compound, wherein the linker group (e.g., “L” or “Link”) is selected from:

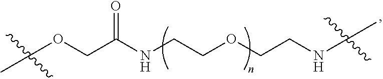

wherein n is from 1-5;

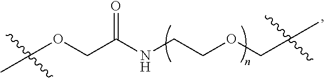

wherein n is from 1-5;

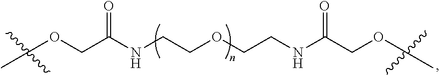

wherein n is from 1-5;

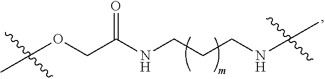

wherein m is from 0-12;

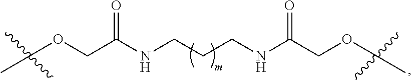

wherein m is from 0-12;

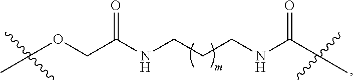

wherein m is from 0-12;

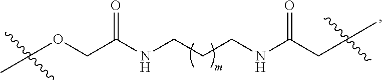

wherein m is from 2-4;

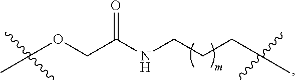

wherein m is from 0-12;

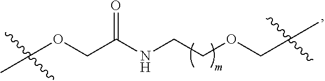

wherein m is from 0-10;

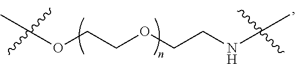

wherein n is from 1-5;

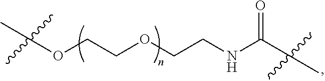

wherein n is from 1-5;

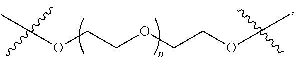

wherein n is from 1-5;

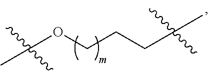

wherein m is from 0-10;

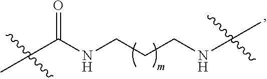

wherein m is from 0-10;

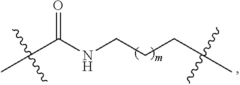

wherein m is from 0-10;

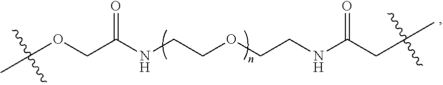

wherein n is from 1-5;

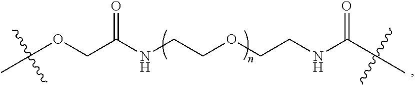

wherein n is from 1-5;

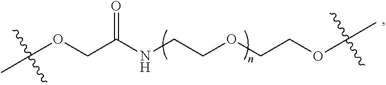

wherein n is from 1-5;

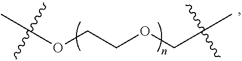

wherein n is from 1-5;

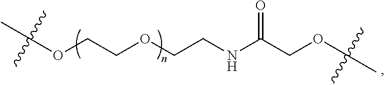

wherein n is from 1-5;

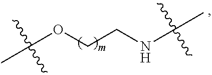

wherein m is from 1-12;

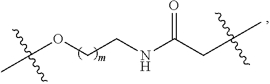

wherein m is from 1-12;

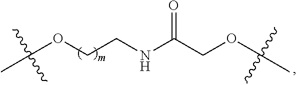

wherein m is from 1-12; and

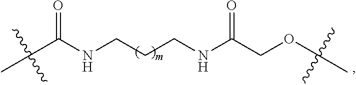

wherein m is from 0-10.
1.35 Any preceding compound, wherein the linker group (e.g., “L” or “Link”) is selected from:

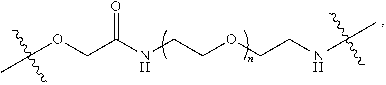

wherein n is from 2-4;

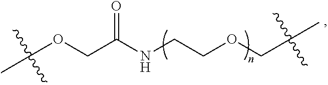

wherein n is from 2-4;

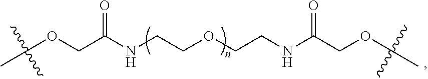

wherein n is from 2-3;

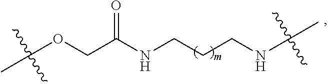

wherein m is from 2-8;

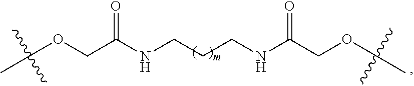

wherein m is from 4-8;

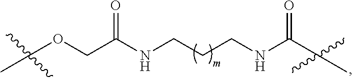

wherein m is from 2-4;

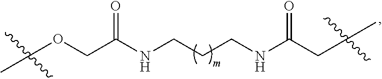

wherein m is from 2-4;

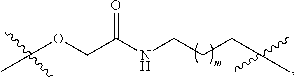

wherein m is from 1-4;

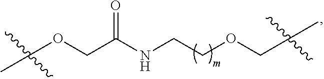

wherein m is from 1-4;

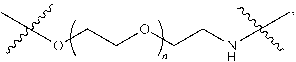

wherein n is from 2-4;

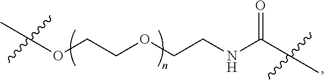

wherein n is from 1-3;

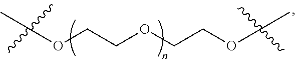

wherein n is from 1-2;

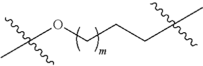

wherein m is from 4-6;

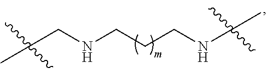

wherein m is from 2-4; and

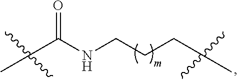

wherein m is from 2-4.
1.36 Any preceding compound, wherein the compound comprises an AR binding moiety as defined in section 1.25, and an E3 ligase binding moiety as defined in section 1.31, and a linker as defined in section 1.34.
1.37 Any preceding compound, wherein the compound comprises an AR binding moiety as defined in section 1.26, and an E3 ligase binding moiety as defined in section 1.31, and a linker as defined in section 1.34.
1.38 Any preceding compound, wherein the compound comprises an AR binding moiety as defined in section 1.25, and an E3 ligase binding moiety as defined in section 1.31, and a linker as defined in section 1.35.
1.39 Any preceding compound, wherein the compound comprises an AR binding moiety as defined in section 1.26, and an E3 ligase binding moiety as defined in section 1.31, and a linker as defined in section 1.35.
1.40 Any preceding compound, wherein the compound comprises:
   the AR binding moiety

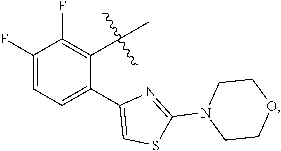 [SMILES: *c1c(-c2csc(N3CCOCC3)n2)ccc(F)c1F]

the E3 ligase binding moiety

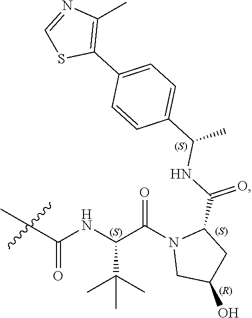

and
a linker selected from:

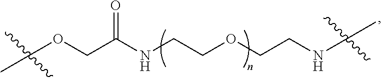

wherein n is from 1-5;

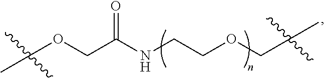

wherein n is from 1-5;

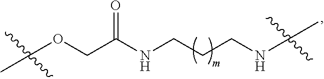

wherein m is from 0-12;

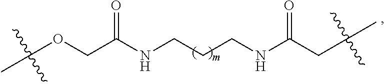

wherein m is from 2-4;

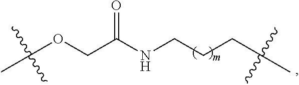

wherein m is from 0-12; and

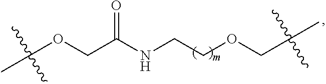

wherein m is from 0-10.
1.41 Any preceding compound, wherein the compound comprises:
   the AR binding moiety

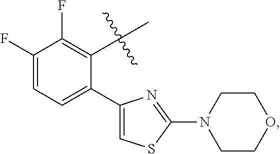 [SMILES: *c1c(-c2csc(N3CCOCC3)n2)ccc(F)c1F]

the E3 ligase binding moiety

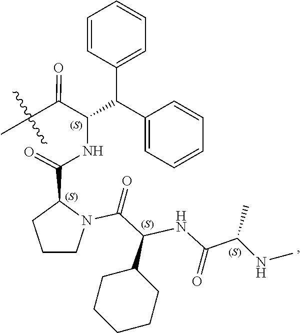

and
a linker selected from:

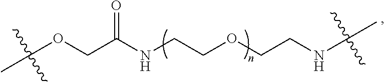

wherein n is from 1-5;

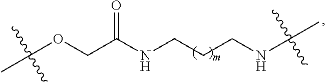

wherein m is from 0-12;

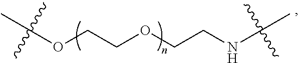

wherein n is from 1-5; and

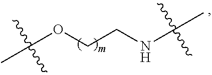

wherein m is from 1-12.
1.42 Any preceding compound, wherein the E3 ligase binding moiety comprises a structure selected from the group consisting of:

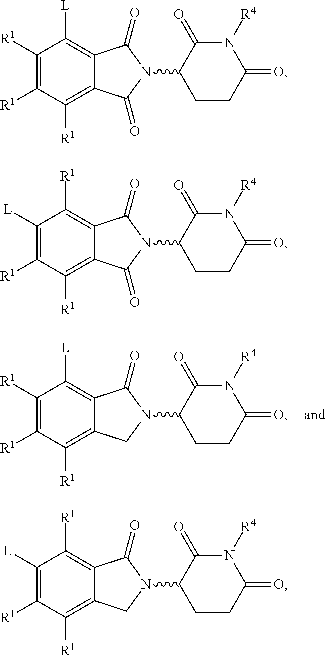

wherein “ [symbol] ” in the above structures, represents a bond that may be stereospecific ((R) or (S)), or non-stereospecific, optionally wherein the bond is oriented to form the (R)-chiral carbon, and wherein:
R¹ is each independently H or halo (e.g., F); and
R⁴ is H;
1.43 Any preceding compound, wherein the compound has an E3 ligase binding moiety selected from:

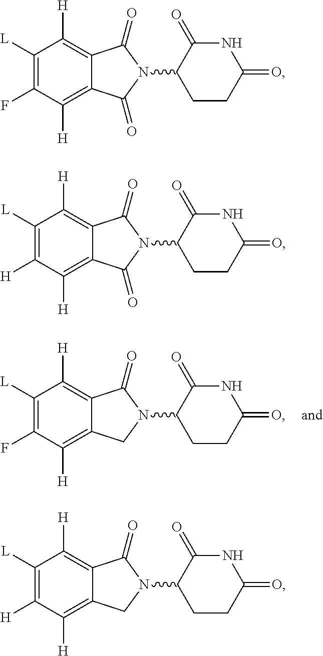

optionally wherein, in each of said compounds, the “ [symbol] ” bond is oriented to form the (R)-chiral carbon.

1.44 Any preceding compound, wherein the compound has an E3 ligase binding moiety selected from:

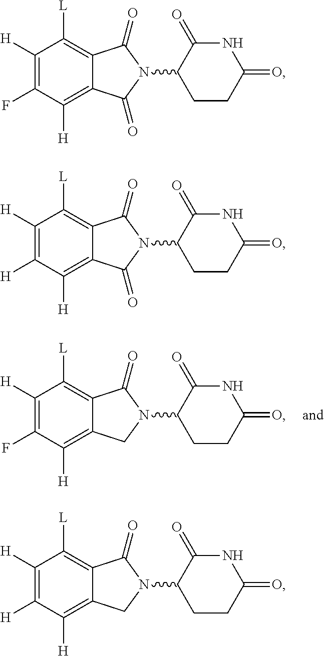

optionally wherein, in each of said compounds, the “ [symbol] ” bond is oriented to form the (R)-chiral carbon.
1.45 Any preceding compound, wherein the linker group (e.g., “L” or “Link”) is selected from:

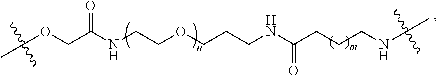

wherein n is from 0-4 (e.g., 1) and m is from 0-4 (e.g., 0);

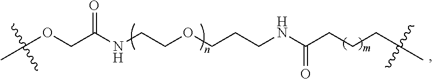

wherein n is from 0-4 (e.g., 1) and m is from 0-4 (e.g., 0);

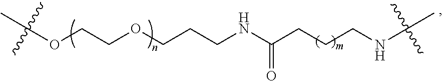

wherein n is from 0-4 (e.g., 1) and m is from 0-4 (e.g., 0);

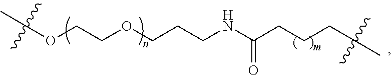

wherein n is from 0-4 (e.g., 1) and m is from 0-4 (e.g., 0);

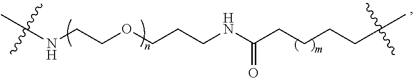

wherein n is from 0-4 (e.g., 1) and m is from 0-4 (e.g., 0);

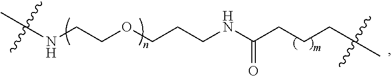

wherein n is from 0-4 (e.g., 1) and m is from 0-4 (e.g., 0);

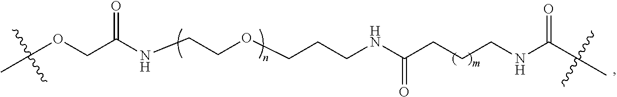

wherein n is from 0-4 (e.g., 1) and m is from 0-4 (e.g., 0);

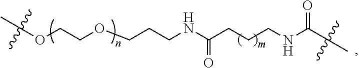

wherein n is from 0-4 (e.g., 1) and m is from 0-4 (e.g., 0);

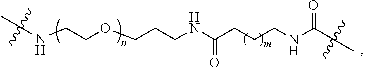

wherein n is from 0-4 (e.g., 1) and m is from 0-4 (e.g., 0);

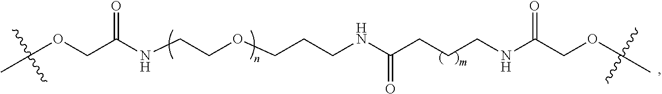

wherein n is from 0-4 (e.g., 1) and m is from 0-4 (e.g., 0);

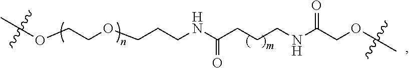

wherein n is from 0-4 (e.g., 1) and m is from 0-4 (e.g., 0);

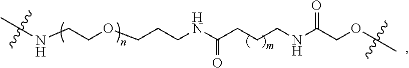

wherein n is from 0-4 (e.g., 1) and m is from 0-4 (e.g., 0);

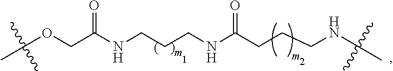

wherein m₁ is from 0-6 (e.g., 0 or 1) and m₂ is from 0-4 (e.g., 0);

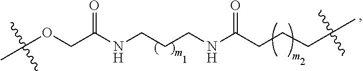

wherein m₁ is from 0-6 (e.g., 0 or 1) and m₂ is from 0-4 (e.g., 0);

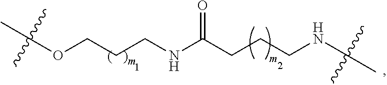

wherein m₁ is from 0-6 (e.g., 0 or 1) and m₂ is from 0-4 (e.g., 0);

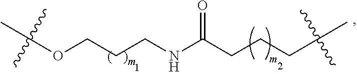

wherein m₁ is from 0-6 (e.g., 0 or 1) and m₂ is from 0-4 (e.g., 0);

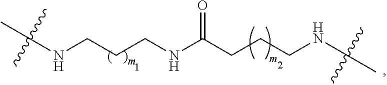

wherein m₁ is from 0-6 (e.g., 0 or 1) and m₂ is from 0-4 (e.g., 0);

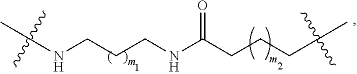

wherein m₁ is from 0-6 (e.g., 0 or 1) and m₂ is from 0-4 (e.g., 0);

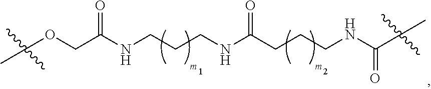

wherein m₁ is from 0-6 (e.g., 0 or 1) and m₂ is from 0-4 (e.g., 0);

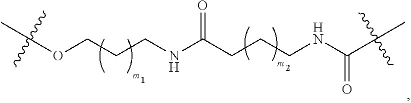

wherein m₁ is from 0-6 (e.g., 0 or 1) and m₂ is from 0-4 (e.g., 0);

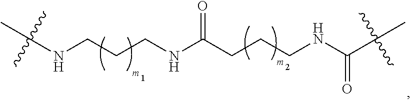

wherein m₁ is from 0-6 (e.g., 0 or 1) and m₂ is from 0-4 (e.g., 0);

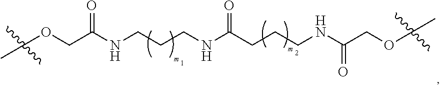

wherein m₁ is from 0-6 (e.g., 0 or 1) and m₂ is from 0-4 (e.g., 0);

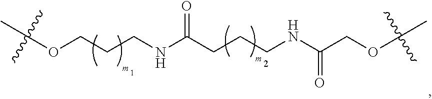

wherein m₁ is from 0-6 (e.g., 0 or 1) and m₂ is from 0-4 (e.g., 0);

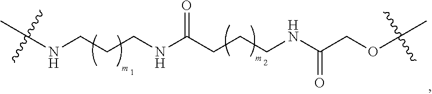

wherein m₁ is from 0-6 (e.g., 0 or 1) and m₂ is from 0-4 (e.g., 0);
1.46 Any preceding compound, wherein the linker group (e.g., “L” or “Link”) is selected from:

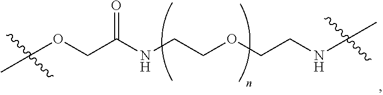

wherein n is from 1 to 4 (e.g., 2, 3, or 4);

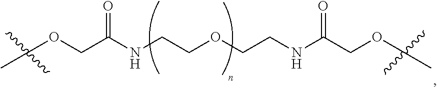

wherein n is from 1 to 4 (e.g., 2, 3, or 4);

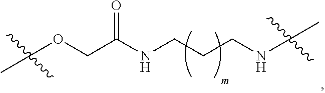

wherein m is from 2 to 8 (e.g., 4, 6, or 8);

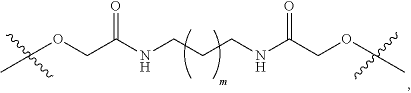

wherein m is from 2 to 8 (e.g., 4, 6, or 8);

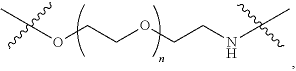

wherein n is from 1 to 4 (e.g., 2, 3, or 4);

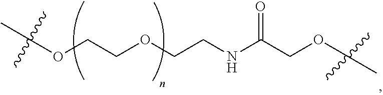

wherein n is from 1 to 4 (e.g., 2, 3, or 4);

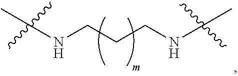

wherein m is from 2 to 8 (e.g., 4, 6, or 8);

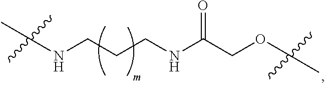

wherein m is from 2 to 8 (e.g., 4, 6, or 8);

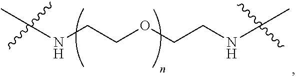

wherein n is from 1 to 4 (e.g., 2, 3, or 4); and

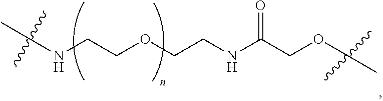

wherein n is from 1 to 4 (e.g., 2, 3, or 4).
1.47 Compound 1.42, wherein the compound comprises:
   the AR binding moiety

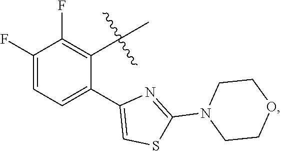 [SMILES: *c1c(-c2csc(N3CCOCC3)n2)ccc(F)c1F]

and
wherein the compound has an E3 ligase binding moiety selected from:

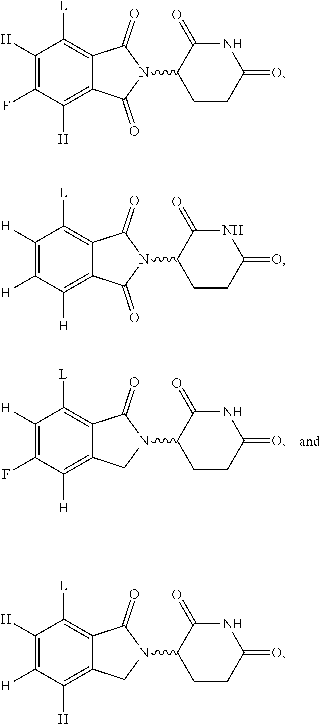

optionally wherein, in each of said compounds, the “ [symbol] ” bond is oriented to form the (R)-chiral carbon.
1.48 Any preceding compound, wherein the linker group (e.g., “L” or “Link”) is selected from:

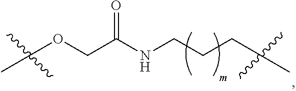

wherein m is from 0-6 (e.g., 1, 2, 3, or 4);

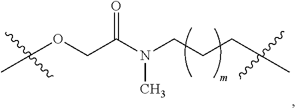

wherein m is from 0-6 (e.g., 1, 2, 3, or 4);

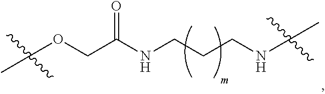

wherein m is from 0-6 (e.g., 1, 2, 3, or 4);

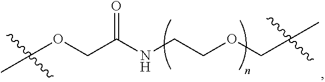

wherein n is from 1-5 (e.g., 1 or 2);

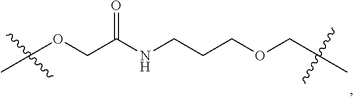

wherein n is from 1-5;

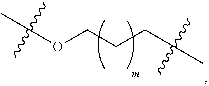

wherein m is from 0-6 (e.g., 2, 3, 4, or 5);

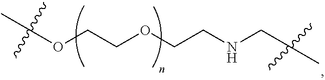

wherein n is from 1-6 (e.g., 1, 2, or 3);

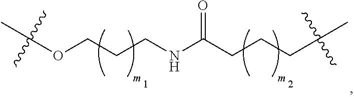

wherein m₁ is from 0-6 (e.g., 1) and m₂ is from 0-4 (e.g., 0); and

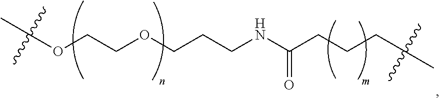

wherein n is from 0-4 (e.g., 1) and m is from 0-4 (e.g., 0).
1.49 Compound 1.48, wherein the compound comprises:
   the AR binding moiety

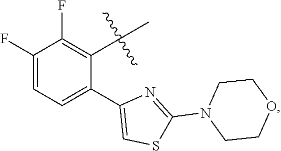 [SMILES: *c1c(-c2csc(N3CCOCC3)n2)ccc(F)c1F]

and
wherein the compound has the E3 ligase binding moiety

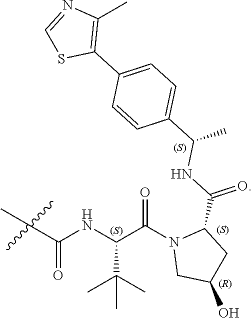

1.50 Compound 1.48, wherein the compound comprises:
   the AR binding moiety

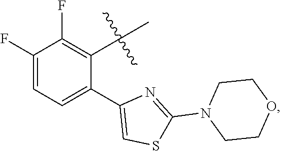 [SMILES: *c1c(-c2csc(N3CCOCC3)n2)ccc(F)c1F]

and
wherein the compound has the E3 ligase binding moiety

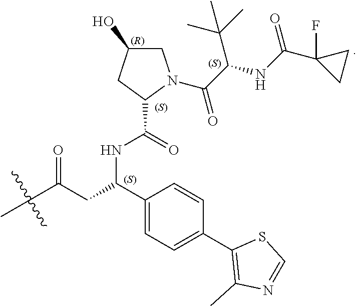

1.51 Any preceding compound, wherein the linker group (e.g., “L” or “Link”) is selected from:

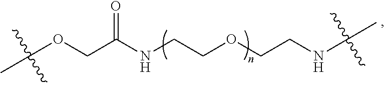

wherein n is from 1 to 4 (e.g., 1, 2, or 3);

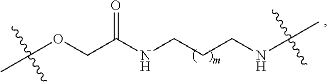

wherein m is from 2 to 8 (e.g., 2, 4, or 6);

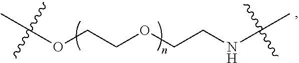

wherein n is 1 to 4 (e.g., 2 or 3); and

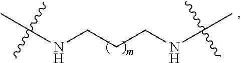

wherein m is from 2 to 8 (e.g., 2, 4, or 6).
1.52 Compound 1.48, wherein the compound comprises:
   the AR binding moiety

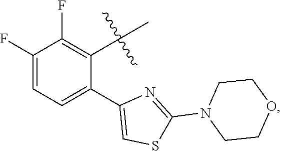 [SMILES: *c1c(-c2csc(N3CCOCC3)n2)ccc(F)c1F]

and

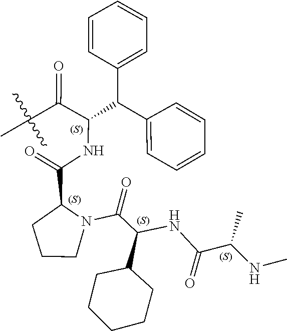

wherein the compound has the E3 ligase binding moiety
1.53 Any preceding compound, wherein the compound is selected from any one or more of Examples 1 to 43 in the table above.
1.54 Any preceding compound, wherein the compound is selected from the group consisting of:
(a) 2-(2,3-difluoro-6-(2-morpholinothiazol-4-yl)phenoxy)-N-(6-((2-(2,6-dioxopiperidin-3-yl)-1,3-dioxoisoindolin-4-yl)amino)hexyl)acetamide,
(b) 2-(2,3-difluoro-6-(2-morpholinothiazol-4-yl)phenoxy)-N-(10-((2-(2,6-dioxopiperidin-3-yl)-1,3-dioxoisoindolin-4-yl)amino)decyl)acetamide,
(c) (2S,4R)-1-((S)-2-(4-(2-(2,3-difluoro-6-(2-morpholinothiazol-4-yl)phenoxy)acetamido)butanamido)-3,3-dimethylbutanoyl)-4-hydroxy-N—((S)-1-(4-(4-methylthiazol-5-yl)phenyl)ethyl)pyrrolidine-2-carboxamide,
(d) (2S,4R)-1-((S)-2-(5-(2-(2,3-difluoro-6-(2-morpholinothiazol-4-yl)phenoxy)acetamido)pentanamido)-3,3-dimethylbutanoyl)-4-hydroxy-N—((S)-1-(4-(4-methylthiazol-5-yl)phenyl)ethyl)pyrrolidine-2-carboxamide,
(e) (2S,4R)-1-((S)-2-(6-(2-(2,3-difluoro-6-(2-morpholinothiazol-4-yl)phenoxy)acetamido)hexanamido)-3,3-dimethylbutanoyl)-4-hydroxy-N—((S)-1-(4-(4-methylthiazol-5-yl)phenyl)ethyl)pyrrolidine-2-carboxamide,
(f) (2S,4R)-1-((S)-2-(7-(2-(2,3-difluoro-6-(2-morpholinothiazol-4-yl)phenoxy)acetamido)heptanamido)-3,3-dimethylbutanoyl)-4-hydroxy-N—((S)-1-(4-(4-methylthiazol-5-yl)phenyl)ethyl)pyrrolidine-2-carboxamide,
(g) (2S,4R)-1-((S)-2-(2-(3-(2-(2,3-difluoro-6-(2-morpholinothiazol-4-yl)phenoxy)acetamido)propoxy)acetamido)-3,3-dimethylbutanoyl)-4-hydroxy-N—((S)-1-(4-(4-methylthiazol-5-yl)phenyl)ethyl)pyrrolidine-2-carboxamide,
(h) (2S,4R)-1-((S)-2-(tert-butyl)-14-(2,3-difluoro-6-(2-morpholinothiazol-4-yl)phenoxy)-4,13-dioxo-6,9-dioxa-3,12-diazatetradecanoyl)-4-hydroxy-N—((S)-1-(4-(4-methylthiazol-5-yl)phenyl)ethyl)pyrrolidine-2-carboxamide,
(i) (2S,4R)-1-((S)-2-(8-(2,3-difluoro-6-(2-morpholinothiazol-4-yl)phenoxy)octanamido)-3,3-dimethylbutanoyl)-4-hydroxy-N—((S)-1-(4-(4-methylthiazol-5-yl)phenyl)ethyl)pyrrolidine-2-carboxamide,
(j) (S)-1-((S)-2-cyclohexyl-2-((S)-2-(methylamino)propanamido)acetyl)-N—((S)-1-((2-(2-(2-(2,3-difluoro-6-(2-morpholinothiazol-4-yl)phenoxy)ethoxy)ethoxy)ethyl)amino)-1-oxo-3,3-diphenylpropan-2-yl)pyrrolidine-2-carboxamide,
(k) (S)-1-((S)-2-cyclohexyl-2-((S)-2-(methylamino)propanamido)acetyl)-N—((S)-1-((4-(2-(2,3-difluoro-6-(2-morpholinothiazol-4-yl)phenoxy)acetamido)butyl)amino)-1-oxo-3,3-diphenylpropan-2-yl)pyrrolidine-2-carboxamide,
(l) (S)-1-((S)-2-cyclohexyl-2-((S)-2-(methylamino)propanamido)acetyl)-N—((S)-1-((6-(2-(2,3-difluoro-6-(2-morpholinothiazol-4-yl)phenoxy)acetamido)hexyl)amino)-1-oxo-3,3-diphenylpropan-2-yl)pyrrolidine-2-carboxamide,
(m)(2R,3 S,4R,5S)-3-(3-chloro-2-fluorophenyl)-4-(4-chloro-2-fluorophenyl)-4-cyano-N-(4-((2-(2-(2-(2,3-difluoro-6-(2-morpholinothiazol-4-yl)phenoxy)ethoxy)ethoxy)ethyl)carbamoyl)-2-methoxyphenyl)-5-neopentylpyrrolidine-2-carboxamide,
(n) (2R,3S,4R,5S)-3-(3-chloro-2-fluorophenyl)-4-(4-chloro-2-fluorophenyl)-4-cyano-N-(4-((4-(2-(2,3-difluoro-6-(2-morpholinothiazol-4-yl)phenoxy)acetamido)butyl)carbamoyl)-2-methoxyphenyl)-5-neopentylpyrrolidine-2-carboxamide,
(o) (2R,3S,4R,5S)-3-(3-chloro-2-fluorophenyl)-4-(4-chloro-2-fluorophenyl)-4-cyano-N-(4-((6-(2-(2,3-difluoro-6-(2-morpholinothiazol-4-yl)phenoxy)acetamido)hexyl)carbamoyl)-2-methoxyphenyl)-5-neopentylpyrrolidine-2-carboxamide,
(p) N-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentyl)-2-morpholinobenzo[d]thiazole-4-carboxamide, and
(q) N-(6-((S)-2-((S)-1-((S)-2-cyclohexyl-2-((S)-2-(methylamino)propanamido)acetyl)pyrrolidine-2-carboxamido)-3,3-diphenylpropanamido)hexyl)-2-morpholinobenzo[d]thiazole-4-carboxamide.
   1.55 Any preceding compound, wherein the compound is selected from:
(a) (2S,4R)-1-((S)-2-(5-(2-(2,3-difluoro-6-(2-morpholinothiazol-4-yl)phenoxy)acetamido)pentanamido)-3,3-dimethylbutanoyl)-4-hydroxy-N—((S)-1-(4-(4-methylthiazol-5-yl)phenyl)ethyl)pyrrolidine-2-carboxamide;
(b) (S)-1-((S)-2-cyclohexyl-2-((S)-2-(methylamino)propanamido)acetyl)-N—((S)-1-((2-(2-(2-(2,3-difluoro-6-(2-morpholinothiazol-4-yl)phenoxy)ethoxy)ethoxy)ethyl)amino)-1-oxo-3,3-diphenylpropan-2-yl)pyrrolidine-2-carboxamide; and
(c) (S)-1-((S)-2-cyclohexyl-2-((S)-2-(methylamino)propanamido)acetyl)-N—((S)-1-((6-(2-(2,3-difluoro-6-(2-morpholinothiazol-4-yl)phenoxy)acetamido)hexyl)amino)-1-oxo-3,3-diphenylpropan-2-yl)pyrrolidine-2-carboxamide.
   1.56 Any of Compounds 1.1-1.55 wherein the compound is effective in causing or promoting the degradation of the androgen receptor (AR) in a cell, or of causing or promoting apoptosis in a cell.
   1.57 Compound 1.56, wherein the cell is a cancer cell (e.g., a prostate cancer cell or ovarian cancer cell, for example, castration-resistant prostate cancer (CRPC) cell).
   1.58 Compound 1.56 or 1.57, wherein the cell overexpresses the AR or expresses a mutated AR, such as an AR having a truncated ligand binding domain or absent ligand binding domain.
   1.59 Compound 1.58, wherein the mutant AR is any AR-V1 to AR-V15 splice variant, e.g., the AR-V7 splice variant.
   1.60 A pharmaceutical composition comprising any of Compounds 1.1-1.59 (e.g., an effective amount of any of Compounds 1.1-1.59), and a pharmaceutically acceptable carrier, additive and/or excipient.
   1.61 Pharmaceutical Composition 1.60, further comprising at least one additional anticancer agent.
   1.62 Any of Compounds 1.1-1.59, or pharmaceutical composition 1.60 or 1.61, for use in the treatment of a disease state or condition in a patient wherein dysregulated protein activity is responsible for said disease or condition.
   1.63 Use of any of Compounds 1.1-1.59, or pharmaceutical composition 1.60 or 1.61, in the treatment of a disease state or condition in a patient wherein dysregulated protein activity is responsible for said disease or condition.
   1.64 A Method of treating a disease state or condition in a patient wherein dysregulated protein activity is responsible for said disease or condition, said method comprising administering an effective amount of any of Compounds 1.1-1.59, or pharmaceutical composition 1.60 or 1.61, to a patient in need thereof.
   1.65 Any of the Uses or Methods according to 1.62 to 1.64, wherein the disease or condition is a cancer.
   1.66 Any of the Uses or Methods according to 1.62 to 1.65, wherein the disease or condition is a cancer identified as having a mutation resulting, or expected to result in, overexpression of the androgen receptor.
   1.67 Use or Method 1.66, wherein the cell expresses a mutated androgen receptor, e.g., one in which there is a mutation in the ligand binding domain of the AR.
   1.68 Use or Method 1.67, wherein the ligand binding domain of the AR is truncated or absent.
   1.69 Any of Uses or Methods 1.64-1.68, wherein the cell expresses or overexpresses any AR-V1 to AR-V15 splice variant, e.g., the AR-V7 splice variant.
   1.70 Any of the uses or methods according to 1.66 to 1.69, wherein the cancer is a prostate cancer or ovarian cancer.
   1.71 Use or Method 1.70, wherein the cancer is a prostate cancer, for example, castration-resistant prostate cancer (CRPC).
   1.72 Any of the uses or methods according to 1.62 to 1.73, wherein the disease or condition is not responsive to, or no longer responsive to, treatment with an androgen receptor antagonist (e.g., abiraterone, apalutamide, enzalutamide, or darolutamide).
   1.73 Any of Compounds 1.1-1.59, or pharmaceutical composition 1.60 or 1.61, for use in the degradation of an androgen receptor in a cell, e.g., a mutated AR such as any AR-V1 to AR-V15 splice variant, e.g., the AR-V7 splice variant.
   1.74 Use of any of Compounds 1.1-1.59, or pharmaceutical composition 1.60 or 1.61, in the degradation of an androgen receptor (AR) in a cell, e.g., a mutated AR such as any AR-V1 to AR-V15 splice variant, e.g., the AR-V7 splice variant.
   1.75 A Method of degrading an androgen receptor in a cell, e.g., a mutated AR such as any AR-V1 to AR-V15 splice variant, e.g., the AR-V7 splice variant, said method comprising administering an effective amount of any of Compounds 1.1-1.59, or pharmaceutical composition 1.60 or 1.61, to such cell.
   1.76 Any of Uses or Methods 1.73-1.75, wherein the cell is a cancer cell (e.g., a prostate cancer cell or ovarian cancer cell, for example, castration-resistant prostate cancer (CRPC) cell).
   1.77 Any of Uses or Methods 1.73-1.76, wherein the cell overexpresses the AR or expresses a mutated AR, such as an AR having a truncated ligand binding domain or absent ligand binding domain.
   1.78 Use or Method 1.77, wherein the mutant AR is any AR-V1 to AR-V15 splice variant, e.g., the AR-V7 splice variant.
   1.79 Any of Uses or Methods 1.73-1.78, wherein the AR is resistant to inhibition by an AR antagonist (e.g., abiraterone, apalutamide, enzalutamide, or darolutamide).
   1.80 Any of Compounds 1.1-1.59, or pharmaceutical composition 1.60 or 1.61, for use in inducing apoptosis in a cell, e.g., a cancer cell.
   1.81 Use of any of Compounds 1.1-1.59, or pharmaceutical composition 1.60 or 1.61, in the induction of apoptosis in a cell, e.g., a cancer cell.
   1.82 A Method of inducing apoptosis in a cell, e.g., a cancer cell, said method comprising administering an effective amount of any of Compounds 1.1-1.59, or pharmaceutical composition 1.60 or 1.61, to such cell.
   1.83 Any of Uses or Methods 1.80-1.82, wherein the cell is a prostate cancer cell or ovarian cancer cell (for example, castration-resistant prostate cancer (CRPC) cell).
   1.84 Any of Uses or Methods 1.80-1.83, wherein the cell overexpresses the androgen receptor (AR) or expresses a mutated AR, such as an AR having a truncated ligand binding domain or absent ligand binding domain.
   1.85 Use or Method 1.84, wherein the mutant AR is any AR-V1 to AR-V15 splice variant, e.g., the AR-V7 splice variant.
   1.86 Any of Uses or Methods 1.73-1.85, wherein the cell is from a patient suffering from or diagnosed with cancer.
   1.87 Any of Uses or Methods 1.73-1.85, wherein the cell is in a patient suffering from or diagnosed with cancer.

### Definitions

Unless otherwise defined, all technical and scientific terms used herein have the same meaning as commonly understood by one of ordinary skill in the art to which this disclosure belongs. The terminology used in the description is for describing particular embodiments only and is not intended to be limiting of the invention.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present disclosure. In instances where a term is not specifically defined herein, that term is given an art-recognized meaning by those of ordinary skill applying that term in context to its use in describing the present invention.

The articles “a” and “an” as used herein and in the claims are used herein to refer to one or to more than one (i.e., to at least one) of the grammatical object of the article unless the context clearly indicates otherwise. By way of example, “an element” means one element or more than one element.

The phrase “and/or” as used herein and in the claims should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the term “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The term “about” and the like, as used herein, in association with numeric values or ranges, reflects the fact that there is a certain level of variation that is recognized and tolerated in the art due to practical and/or theoretical limitations. For example, minor variation is tolerated due to inherent variances in the manner in which certain devices operate and/or measurements are taken. In accordance with the above, the term “about” is normally used to encompass values within the standard deviation or standard error.

In the claims, as well as in the specification, all transitional phrases such as “comprising,” “including,” “carrying,” “having,” “containing,” “involving,” “holding,” “composed of,” and the like are to be understood to be open-ended, i.e., to mean “including without limitation”. Only the transitional phrases “consisting of” and “consisting essentially of” shall be closed or semi-closed transitional phrases, respectively, as set forth in the United States Patent Office Manual of Patent Examining Procedures, Section 2111.03.

It should also be understood, that although various compounds, compositions, and methods are described in “open” terms of “comprising,” “including,” or “having” various components or steps (interpreted as meaning “including without limitation”), the compounds, compositions, methods, and devices can also “consist essentially of” or “consist of” the various components and steps, and such terminology should be interpreted as defining essentially closed-member groups. This paragraph is not meant in any way to limit the meaning of “comprising,” “having,” or “including” (and other verb forms thereof) which are to be interpreted as open-ended phrases meaning “including without limitation” consistent with patent law and custom. The intent of this paragraph is merely to indicate that the closed-member groups defined by the “consisting of” or “consisting essentially of” language are lesser included groups within the open-ended descriptions and to provide support for claims employing the “consisting of” or “consisting essentially of” language.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration” and “co-administering” or “combination therapy” can refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are co-administered in combination with at least one additional bioactive agent, especially including an anticancer agent. In particularly preferred aspects, the co-administration of compounds results in synergistic activity and/or therapy, including anticancer activity.

The term “effective” can mean, but is in no way limited to, that amount/dose of the active pharmaceutical ingredient, which, when used in the context of its intended use, effectuates or is sufficient to prevent, inhibit the occurrence, ameliorate, delay or treat (alleviate a symptom to some extent, preferably all) the symptoms of a condition, disorder or disease state in a subject in need of such treatment or receiving such treatment. The term effective subsumes all other effective amount or effective concentration terms, e.g., “effective amount/dose,” “pharmaceutically effective amount/dose” or “therapeutically effective amount/dose,” which are otherwise described or used in the present application.

The effective amount depends on the type and severity of disease, the composition used, the route of administration, the type of mammal being treated, the physical characteristics of the specific mammal under consideration, concurrent medication, and other factors which those skilled in the medical arts will recognize. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington, The Science and Practice of Pharmacy, 20th Edition, 2003, Gennaro, Ed., Lippincott, Williams & Wilkins).

The term “pharmacological composition,” “therapeutic composition,” “therapeutic formulation” or “pharmaceutically acceptable formulation” can mean, but is in no way limited to, a composition or formulation that allows for the effective distribution of an agent provided by the present disclosure, which is in a form suitable for administration to the physical location most suitable for their desired activity, e.g., systemic administration.

The term “pharmaceutically acceptable” can mean, but is in no way limited to, entities and compositions that do not produce an adverse, allergic or other untoward reaction when administered to a patient or subject.

The term “pharmaceutically acceptable carrier” can mean, but is in no way limited to, any and all solvents, dispersion media, coatings, antibacterial and antifungal agents, isotonic and absorption delaying agents, and the like, compatible with pharmaceutical administration to a patient or subject. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “systemic administration” refers to a route of administration that is, e.g., enteral or parenteral, and results in the systemic distribution of an agent leading to systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect. Administration routes which lead to systemic absorption include, without limitations: intravenous, subcutaneous, intraperitoneal, inhalation, oral, intrapulmonary and intramuscular.

The rate of entry of a drug into the circulation has been shown to be a function of molecular weight or size. The use of a liposome or other drug carrier comprising the compounds of the instant disclosure can potentially localize the drug, for example, in certain tissue types, such as the tissues of the reticular endothelial system (RES). A liposome formulation which can facilitate the association of drug with the surface of cells, such as, lymphocytes and macrophages is also useful.

The terms “patient” and “subject” are used throughout the specification to describe a cell, tissue, or animal, preferably a mammal, e.g., a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “compound,” as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. The term also refers to any specific chemical compound in which one or more atoms have been replaced with one or more different isotopes of the same element. It is noted that in describing the present compounds, numerous substituents and variables associated with same, among others, are described.

It is understood by those of ordinary skill that molecules which are described herein are stable compounds as generally described hereunder. When the bond is shown, both a double bond and single bond are represented or understood within the context of the compound shown and well-known rules for valence interactions.

As used herein, “derivatives” can mean compositions formed from the native compounds either directly, by modification, or by partial substitution. As used herein, “analogs” can mean compositions that have a structure similar to, but not identical to, the native compound.

The term “ubiquitin ligase” refers to a family of proteins that facilitate the transfer of ubiquitin to a specific substrate protein, targeting the substrate protein for degradation. For example, cereblon is an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase may be involved in polyubiquitination such that a second ubiquitin may be attached to the first; a third may be attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins may not be targeted to the proteasome for degradation, but may instead be altered in their cellular location or function, for example, via binding other proteins that have domains capable of binding ubiquitin. Further, different lysine residues on ubiquitin can be targeted by an E3 to make chains. The most common lysine is Lys48 on the ubiquitin chain. This is the lysine used to make polyubiquitin, which is recognized by the proteasome.

As used herein, the terms “halo” or “halogen” means fluoro (F), chloro (Cl), bromo (Br) or iodo (I).

As used herein, the term “hydrocarbyl” means a compound which contains carbon and hydrogen and which may be fully saturated, partially unsaturated or aromatic and includes aryl groups, alkyl groups, alkenyl groups and alkynyl groups.

As used herein, the term “alkyl” means within its context a linear, branch-chained, or cyclic fully saturated hydrocarbon radical or alkyl group, preferably a C₁-C₁₀, more preferably a C₁-C₆, alternatively a C₁-C₃ alkyl group, which may be optionally substituted. Examples of alkyl groups are methyl, ethyl, n-butyl, sec-butyl, n-hexyl, n-heptyl, n-octyl, n-nonyl, n-decyl, isopropyl, 2-methylpropyl, cyclopropyl, cyclopropylmethyl, cyclobutyl, cyclopentyl, cyclopentylmethyl, cyclopentylethyl, cyclohexylmethyl, cyclohexylethyl and cyclohexyl, among others.

As used herein, the term “alkenyl” refers to linear, branched or branch-chained, or cyclic C₂-C₁₀ (preferably C₂-C₆) hydrocarbon radicals containing at least one C≡C bond for example, vinyl or allyl.

As used herein, the term “Alkynyl” refers to linear, branched or branch-chained, or cyclic C₂-C₁₀ (preferably C₂-C₆) hydrocarbon radicals containing at least one C≡C bond, for example, propargyl.

As used herein, the term “alkylene” refers to a —(CH₂)ₙ— group (wherein n is an integer generally from 0-6), which may be optionally substituted. When substituted, the alkylene group preferably is substituted on one or more of the methylene groups with a C₁-C₆ alkyl group (including a cyclopropyl group or a t-butyl group), more preferably a methyl group, but may also be substituted with one or more halo groups, preferably from 1 to 3 halo groups or one or two hydroxyl groups, O—(C₁-C₆ alkyl) groups or amino acid sidechains as otherwise disclosed herein. In certain embodiments, an alkylene group may be substituted with a urethane or alkoxy group (or other group) which is further substituted with a polyethylene glycol chain (of from 1 to 10, preferably 1 to 6, often 1 to 4 ethylene glycol units) to which is substituted (preferably, but not exclusively on the distal end of the polyethylene glycol chain) an alkyl chain substituted with a single halogen group, preferably a chlorine group. In still other embodiments, the alkylene (often, a methylene) group, may be substituted with an amino acid sidechain group such as a sidechain group of a natural or unnatural amino acid, for example, alanine, (3-alanine, arginine, asparagine, aspartic acid, cysteine, cystine, glutamic acid, glutamine, glycine, phenylalanine, histidine, isoleucine, lysine, leucine, methionine, praline, serine, threonine, valine, tryptophan, or tyrosine.

As used herein, a range of carbon atoms which includes C₀ means that carbon is absent and is replaced with H (or deuterium). Thus, a range of carbon atoms which is C₀-C₆ includes carbons atoms of 1, 2, 3, 4, 5 and 6 and for C₀, H (or deuterium) stands in place of carbon.

As used herein, the term “unsubstituted” means substituted only with hydrogen atoms.

As used herein, the term “substituted” or “optionally substituted” means independently (i.e., where more than a single substitution occurs, each substituent is independent of another substituent) one or more substituents (independently up to five substituents, preferably up to three substituents, often 1 or 2 substituents on a moiety in a compound according to the present invention, and may include substituents which themselves may be further substituted) at a carbon (or nitrogen) position anywhere on a molecule within context, and includes as substituents hydroxyl, thiol, carboxyl, cyano (C≡N), nitro (NO₂), halogen (preferably, 1, 2 or 3 halogens, especially on an alkyl, especially a methyl group such as a trifluoromethyl), an alkyl group (preferably, C₁-C₁₀, more preferably, C₁-C₆), aryl (especially phenyl and substituted phenyl for example benzyl or benzoyl), alkoxy group (preferably, C₁-C₆ alkyl or aryl, including phenyl and substituted phenyl), thioether (C₁-C₆ alkyl or aryl), acyl (preferably, C₁-C₆ acyl), ester or thioester (preferably, C₁-C₆ alkyl or aryl) including alkylene ester (such that attachment is on the alkylene group, rather than at the ester function which is preferably substituted with a C₁-C₆ alkyl or aryl group), preferably, C₁-C₆ alkyl or aryl, halogen (preferably, F or Cl), amine (including a five- or six-membered cyclic alkylene amine, further including a C₁-C₆ alkyl amine or a C₁-C₆ dialkyl amine which alkyl groups may be substituted with one or two hydroxyl groups) or an optionally substituted N(C₀-C₆ alkyl)C(O)(OC₁-C₆ alkyl) group (which may be optionally substituted with a polyethylene glycol chain to which is further bound an alkyl group containing a single halogen, preferably chlorine substituent), hydrazine, amido, which is preferably substituted with one or two C₁-C₆ alkyl groups (including a carboxamide which is optionally substituted with one or two C₁-C₆ alkyl groups), alkanol (preferably, C₁-C₆ alkyl or aryl), or alkanoic acid (preferably, C₁-C₆ alkyl or aryl). Substituents according to the present invention may include, for example SiR₁R₂R₃ groups wherein each of R₁ and R₂ is as otherwise described herein, and R₃ is H or a C₁-C₆ alkyl group, preferably R₁, R₂, R₃ in this context is a C₁-C₃ alkyl group (including an isopropyl or t-butyl group). Each of the above-described groups may be linked directly to the substituted moiety or alternatively, the substituent may be linked to the substituted moiety (preferably in the case of an aryl or heteroaryl moiety) through an optionally substituted —(CH₂)ₘ— or, alternatively, an optionally substituted —(OCH₂)ₘ—, —(OCH₂CH₂)ₘ— or —(CH₂CH₂O)ₘ— group, which may be substituted with any one or more of the above-described substituents. Alkylene groups —(CH₂)ₘ— or —(CH₂)ₙ— groups or other chains such as ethylene glycol chains, as identified above, may be substituted anywhere on the chain.

Preferred substituents on alkylene groups include halogen or C₁-C₆ (preferably C₁-C₃) alkyl groups, which may be optionally substituted with one or two hydroxyl groups, one or two ether groups (O—C₁-C₆ groups), up to three halo groups (preferably F), or a sidechain of an amino acid as otherwise described herein and optionally substituted amide (preferably carboxamide substituted as described above) or urethane groups (often with one or two C₀-C₆ alkyl substituents, which group(s) may be further substituted). In certain embodiments, the alkylene group (often a single methylene group) is substituted with one or two optionally substituted C₁-C₆ alkyl groups, preferably C₁-C₄ alkyl group, most often methyl or O-methyl groups or a sidechain of an amino acid as otherwise described herein. In the present invention, a moiety in a molecule may be optionally substituted with up to five substituents, preferably up to three substituents. Most often, in the present invention moieties which are substituted are substituted with one or two substituents.

As used herein, the term “substituted” (each substituent being independent of any other substituent) also means within its context of use C₁-C₆ alkyl, C₁-C₆ alkoxy, halogen, amido, carboxamido, sulfone, including sulfonamide, keto, carboxy, C₁-C₆ ester (oxy ester or carbonyl ester), C₁-C₆ keto, urethane —O—C(O)—NR₁R₂ or —N(R₁)—C(O)—O—R₁, nitro, cyano and amine (especially including a C₁-C₆ alkylene —NR₁R₂, a mono- or di-C₁-C₆ alkyl substituted amines which may be optionally substituted with one or two hydroxyl groups). Each of these groups contains unless otherwise indicated, within context, between 1 and 6 carbon atoms. In certain embodiments, preferred substituents will include, for example, NH, NHC(O), O, ═O, (CH₂)ₘ (here, m and n are in context, 1, 2, 3, 4, 5 or 6), S, S(O), SO₂ or NHC(O)NH, (CH₂)ₙOH, (CH₂)ₙSH, (CH₂)ₙCOOH, C₁-C₆ alkyl, (CH₂)ₙO(C₁-C₆ alkyl), (CH₂)ₙC(O)(C₁-C₆ alkyl), (CH₂)ₙOC(O)(C₁-C₆ alkyl), (CH₂)ₙC(O)O(C₁-C₆ alkyl), (CH₂)ₙNHC(O)R₁, (CH₂)ₙC(O)NR₁R₂, (OCH₂)ₙOH, (CH₂O)ₙCOOH, C₁-C₆ alkyl, (OCH₂)ₙO(C₁-C₆ alkyl), (CH₂O)ₙC(O)(C₁-C₆ alkyl), (OCH₂)ₙNHC(O)R₁, (CH₂O)ₙC(O)NR₁R², S(O)₂Rₛ, S(O)Rₛ (Rₛ is C₁-C₆ alkyl or a (CH₂)ₘNR₁R₂ group), NO₂, CN, or halogen (F, Cl, Br, I, preferably F or Cl), depending on the context of the use of the substituent. R₁ and R₂ are each, within context, H or a C₁-C₆ alkyl group (which may be optionally substituted with one or two hydroxyl groups or up to three halogen groups, preferably fluorine).

The term “substituted” also means, within the chemical context of the compound defined and substituent used, an optionally substituted aryl or heteroaryl group or an optionally substituted heterocyclic group as otherwise described herein. Alkylene groups may also be substituted as otherwise disclosed herein, preferably with optionally substituted C₁-C₆ alkyl groups (methyl, ethyl or hydroxymethyl or hydroxyethyl is preferred, thus providing a chiral center), a sidechain of an amino acid group as otherwise described herein, an amido group as described hereinabove, or a urethane group OC(O)NR₁R₂ group wherein R₁ and R₂ are as otherwise described herein, although numerous other groups may also be used as substituents. Various optionally substituted moieties may be substituted with 3 or more substituents, preferably no more than 3 substituents and preferably with 1 or 2 substituents. It is noted that in instances where, in a compound at a particular position of the molecule substitution is required (principally, because of valency), but no substitution is indicated, then that substituent is construed or understood to be H, unless the context of the substitution suggests otherwise.

As used herein, the terms “aryl” and “aromatic,” in context, refer to a substituted (as otherwise described herein) or unsubstituted monovalent aromatic radical having a single ring (e.g., benzene, phenyl, benzyl) or condensed rings (e.g., naphthyl, anthracenyl, phenanthrenyl, etc.) and can be bound to the compound according to the present invention at any available stable position on the ring(s) or as otherwise indicated in the chemical structure presented. Other examples of aryl groups, in context, may include heterocyclic aromatic ring systems “heteroaryl” groups having one or more nitrogen, oxygen, or sulfur atoms in the ring (monocyclic) such as imidazole, furyl, pyrrole, furanyl, thiene, thiazole, pyridine, pyrimidine, pyrazine, triazole, oxazole or fused ring systems such as indole, quinoline, indolizine, azaindolizine, benzofurazan, etc., among others, which may be optionally substituted as described above. Among the heteroaryl groups which may be mentioned include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, azaindolizine, purine, indazole, quinoline, dihydroquinoline, tetrahydroquinoline, isoquinoline, dihydroisoquinoline, tetrahydroiso-quinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, perimidine, phenanthroline, phenacene, oxadiazole, benzimidazole, pyrrolopyridine, pyrrolopyrimidine and pyridopyrimidine; sulfur containing aromatic heterocycles such as thiophene and benzothiophene; oxygen containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadiazole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

As used herein, the term “substituted aryl” refers to an aromatic carbocyclic group comprised of at least one aromatic ring or of multiple condensed rings at least one of which being aromatic, wherein the ring(s) are substituted with one or more substituents. For example, an aryl group can comprise a substituent(s) selected from: (CH₂)ₙOH, (CH₂)ₙO(C₁-C₆)alkyl, (CH₂)ₙO(CH₂)ₙ(C₁-C₆)alkyl, (CH₂)ₙC(O)(C₀-C₆) alkyl, (CH₂)ₙC(O)O(C₀-C₆) alkyl, (CH₂)ₙOC(O)(C₀-C₆) alkyl, amine, mono- or di-(C₁-C₆ alkyl) amine wherein the alkyl group on the amine is optionally substituted with 1 or 2 hydroxyl groups or up to three halo (preferably F, Cl) groups, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is preferably substituted with a linker group attached to a ARB group, including a E3LB group), and/or at least one of F, Cl, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methyl substituted isoxazole, an optionally substituted oxazole including a methyl substituted oxazole, an optionally substituted thiazole including a methyl substituted thiazole, an optionally substituted isothiazole including a methyl substituted isothiazole, an optionally substituted pyrrole including a methyl substituted pyrrole, an optionally substituted imidazole including a methyl imidazole, an optionally substituted benzimidazole or methoxybenzyl-imidazole, an optionally substituted oximidazole or methyloximidazole, an optionally substituted diazole group, including a methyldiazole group, an optionally substituted triazole group, including a methyl substituted triazole group, an optionally substituted pyridine group, including a halo (preferably, F) or methyl substituted pyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, and combinations thereof.

As used herein, the term “carboxyl” denotes the group C(O)OR, wherein R is hydrogen, alkyl, substituted alkyl, aryl, substituted aryl, heteroaryl or substituted heteroaryl, whereas these generic substituents have meanings which are identical with definitions of the corresponding groups defined herein.

As used herein, the terms “heteroaryl” and “hetaryl” include, without limitation, an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted (CH₂)ₘOC₁-C₆ alkyl group or an optionally substituted (CH₂)ₘC(O)OC₁-C₆ alkyl group), an optionally substituted pyridine (2-, 3-, or 4-pyridine) or a group according to the chemical structure:

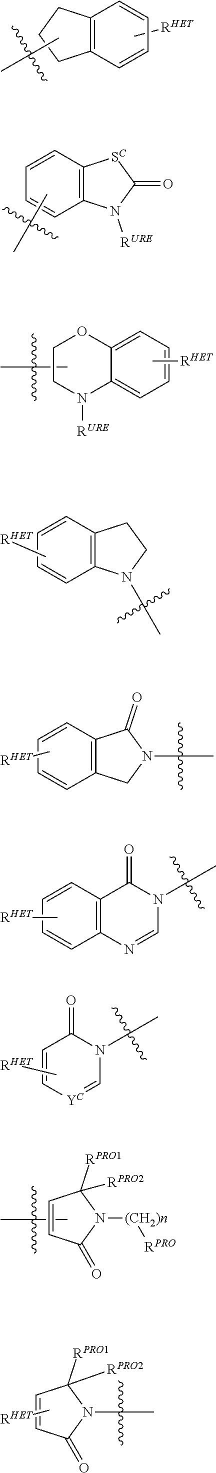

wherein S^{C} is CHR^{SS}, NR^{URE} or O;
R^{HET} is H, CN, NO₂, halo (preferably Cl or F), optionally substituted C₁-C₆ alkyl (preferably substituted with one or two hydroxyl groups or up to three halo groups (e.g., CF₃), optionally substituted O(C₁-C₆ alkyl) (preferably substituted with one or two hydroxyl groups or up to three halo groups) or an optionally substituted acetylenic group

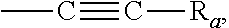

wherein Rₐ is H or a C₁-C₆ alkyl group (preferably C₁-C₃ alkyl). R^{SS} is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} is H, a C₁-C₆ alkyl (preferably H or C₁-C₃ alkyl) or a —C(O)(C₁-C₆ alkyl), each of which groups is optionally substituted with one or two hydroxyl groups or up to three halogens, preferably fluorine groups, or an optionally substituted phenyl group, an optionally substituted heterocycle, for example piperidine, morpholine, pyrrolidine, tetrahydrofuran, tetrahydrothiophene, piperidine, piperazine, each of which is optionally substituted, and
Y^{C} is N or C—R^{YC}, wherein R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group

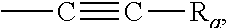

wherein Rₐ is H or a C₁-C₆ alkyl group (preferably C₁-C₃ alkyl).

R^{PRO} is H, optionally substituted C₀-C₆ alkyl or an optionally substituted aryl, heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline (each preferably substituted with a C₀-C₃ alkyl group, preferably methyl or a halo group preferably F or Cl), benzofuran, indole, indolizine, azaindolizine: R^{PRO1} and R_{PRO2} are each independently H, an optionally substituted C₀-C₃ alkyl group or together form a keto group and each n is independently 0, 1, 2, 3, 4, 5 or 6 or an optionally substituted heterocycle, preferably tetrahydrofuran, tetrahydrothiene, piperidine, piperazine or morpholine (each of which groups when substituted are preferably substituted with a methyl or halo).

As used herein, the terms “arylalkyl” and “heteroarylalkyl” refer to groups that comprise both aryl or, respectively, heteroaryl as well as alkyl and/or heteroalkyl and/or carbocyclic and/or heterocycloalkyl ring systems according to the above definitions.

As used herein, the term “arylalkyl” as used herein refers to an aryl group as defined above appended to an alkyl group defined above. The arylalkyl group is attached to the parent moiety through an alkyl group wherein the alkyl group is one to six carbon atoms. The aryl group in the arylalkyl group may be substituted as defined above.

As used herein, the terms “heterocycle” and “heterocyclic” refer to a cyclic group which contains at least one heteroatom, i.e., O, N or S, and may be aromatic (heteroaryl) or non-aromatic. Thus, the heteroaryl moieties are subsumed under the definition of heterocycle, depending on the context of its use. Exemplary heterocycles include: azetidinyl, benzimidazolyl 1,4-benzodioxanyl, 1,3-benzodioxolyl, benzoxazolyl, benzothiazolyl, benzothienyl, dihydroimidazolyl, dihydropyranyl, dihydrofuranyl, dioxanyl, dioxolanyl, ethyleneurea, 1,3-dioxolane, 1,3-dioxane, 1,4-dioxane, furyl, homopiperidinyl, imidazolyl, imidazolinyl, imidazolidinyl, indolinyl, indolyl, isoquinolinyl, isothiazolidinyl, isothiazolyl, isoxazolidinyl, isoxazolyl, morpholinyl, naphthyridinyl, oxazolidinyl, oxazolyl, pyridone, 2-pyrrolidone, pyridine, piperazinyl, N-methylpiperazinyl, piperidinyl, phthalimide, succinimide, pyrazinyl, pyrazolinyl, pyridyl, pyrimidinyl, pyrrolidinyl, pyrrolinyl, pyrrolyl, quinolinyl, tetrahydrofuranyl, tetrahydropyranyl, tetrahydroquinoline, thiazolidinyl, thiazolyl, thienyl, tetrahydrothiophene, oxane, oxetanyl, oxathiolanyl, and thiane among others.

Heterocyclic groups can be optionally substituted with a member selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxy, carboxyalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SOaryl, —SO-heteroaryl, —SO₂-alkyl, —SO₂-substituted alkyl, —SO₂-aryl, oxo (═O), and —SO₂-heteroaryl. Such heterocyclic groups can have a single ring or multiple condensed rings. Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy nitrogen containing heterocycles. The term “heterocyclic” also includes bicyclic groups in which any of the heterocyclic rings is fused to a benzene ring or a cyclohexane ring or another heterocyclic ring (for example, indolyl, quinolyl, isoquinolyl, tetrahydroquinolyl, and the like).

As used herein, the term “cycloalkyl” includes, without limitation, univalent groups derived from monocyclic or polycyclic alkyl groups or cycloalkanes, as defined herein, e.g., saturated monocyclic hydrocarbon groups having from three to twenty carbon atoms in the ring, including, but not limited to, cyclopropyl, cyclobutyl, cyclopentyl, cyclohexyl, cycloheptyl and the like.

As used herein, the term “substituted cycloalkyl” includes, without limitation, a monocyclic or polycyclic alkyl group being substituted by one or more substituents, for example, amino, halogen, alkyl, substituted alkyl, carbyloxy, carbylmercapto, aryl, nitro, mercapto, or sulfa, whereas these generic substituent groups have meanings which are identical with definitions of the corresponding groups as defined herein.

As used herein, the term “heterocycloalkyl” refers to a monocyclic or polycyclic alkyl group in which at least one ring carbon atom of its cyclic structure being replaced with a heteroatom selected from the group consisting of N, O, S, or P.

As used herein, the term “substituted heterocycloalkyl” refers to a monocyclic or polycyclic alkyl group in which at least one ring carbon atom of its cyclic structure being replaced with a heteroatom selected from the group consisting of N, O, S, or P, and the group contains one or more substituents selected from the group consisting of halogen, alkyl, substituted alkyl, carbyloxy, carbylmercapto, aryl, nitro, mercapto, or sulfa, whereas these generic substituent group have meanings which are identical with definitions of the corresponding groups as defined herein.

### EXAMPLES

Unless otherwise noted, starting materials, reagents, and solvents were obtained from commercial suppliers (e.g., Acros Organics, Sigma-Aldrich, Alfa Aesar, Fluorochem, and Merck) and were used without further purification. Reactions were routinely monitored by thin-layer chromatography (TLC) performed on silica gel 60 F₂₅₄ (layer 0.2 mm) pre-coated aluminum foil (with fluorescent indicator UV254) (Sigma-Aldrich). Developed plates were air-dried and visualized under UV light (254/365 nm) or by using KMnO₄ or ninhydrin solutions. Flash column chromatography was performed on Merck silica gel 60 (mesh 230-400). Automated flash chromatographic purifications were performed using Biotage® Selekt (Cartridge: Sfär Silica HC Duo 5 g or 10 g). Preparative TLC purification was performed on Merck silica gel 60 F254 (0.5 mm) pre-coated glass plates (20×20 cm) (Sigma-Aldrich).

¹H NMR and ¹³C NMR spectra were recorded at room temperature at 400 and 101 MHz, respectively, on a Bruker Avance 400 spectrometer by using TMS or residual solvent peak as internal standard. Chemical shifts are reported in ppm (δ) and the coupling constants (J) are given in Hertz (Hz). Peak multiplicities are abbreviated as follow: s (singlet), bs (broad singlet), d (doublet), dd (double doublet), t (triplet), dt (double triplet), q (quartet), p (pentet), and m (multiplet).

High-Resolution Mass Spectroscopy (HRMS) spectra were registered on Agilent Technologies 6540 UHD Accurate Mass Q-TOF LC-MS system or on Agilent 1290 Infinity Series U-HPLC system (Agilent Technologies, Santa Clara, Calif., USA) coupled with a Q-TOF 6540 high-resolution mass spectrometer and 1290 Infinity Series DAD/UV-Vis detector (Agilent Technologies). The purity of all final compounds that were evaluated in biological assays was assessed as >95%, using LC-MS. The analyses were carried out according to the method listed below. The mobile phase was a mixture of water (solvent A) and acetonitrile (solvent B), both containing formic acid at 0.1%. Method: Acquity UPLC BEH C18 1.7 μm (C18, 150×2.1 mm) column at 40° C. using a flow rate of 0.65 mL/min in a 10 min gradient elution. Gradient elution was as follows: 99.5:0.5 (A/B) to 5:95 (A/B) over 8 min, 5:95 (A/B) for 2 min, and then reversion back to 99.5:0.5 (A/B) over 0.1 min. The UV detection is an averaged signal from wavelength of 190 nm to 640 nm and mass spectra are recorded on a mass spectrometer using positive mode electro spray ionization. The chemical names were generated using ChemBioDraw 12.0 from CambridgeSoft.

Compounds described herein may be synthesized as described herein, using modified methods described herein or by methods known to a person of skill in the art.

Chemistry abbreviations: ACN, acetonitrile; AcOH, acetic acid; AcOK, potassium acetate; Boc, tert-butoxycarbonyl; CD₃OD, deuterated methanol; CDCl₃, deuterated chloroform; DCE, dichloroethane; DCM, dichloromethane; DEE, diethyl ether; DIAD, diisopropyl azodicarboxylate; DIPEA, N,N′-diisopropylethylamine; DMA, dimethylacetamide; DMF, dimethylformamide; DMSO, dimethylsulfoxide; DMSO-d₆, deuterated dimethylsulfoxide; EA, ethyl acetate; h, hour; EDC, 1-Ethyl-3-(3-dimethylaminopropyl)carbodiimide; Et₃N, triethylamine; HATU, 2-(7-Aza-1H-benzotriazole-1-yl)-1,1,3,3-tetramethyluronium hexafluorophosphate; min, minutes; HOBt, 1-hydroxybenzotriazole; HRMS, high-resolution mass spectroscopy; MeOH, methanol; NMR, nuclear magnetic resonance; tBu, tert-butyl; THF, tetrahydrofuran; TLC, thin-layer chromatography; TMS, tetramethylsilane; PE, petroleum ether; rt, room temperature.

### Chemical Synthesis

Compounds of general formula (I) may be prepared by the general synthetic approaches described below (General Scheme 1 and 2), together with synthetic methods known in the art of organic chemistry. In all methods, it is well-understood that protecting groups for sensitive or reactive groups may be employed where necessary in accordance with general principles of chemistry. Protecting groups are manipulated according to standard methods of organic synthesis (T. W. Green and P. G. M. Wuts (1999) Protective Groups in Organic Synthesis, 3′ edition, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I). Specific detailed synthetic procedures for a variety of intermediates and final compounds within the scope of the present disclosure is publicly available in the U.S. Patent Application Publication of U.S. patent application Ser. No. 16/777,294, filed on Jan. 30, 2020, as well as its PCT counterpart, which are publishing on or about Jul. 30, 2020.

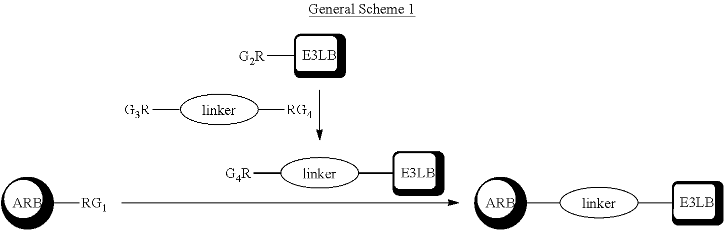

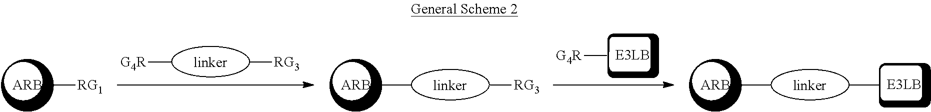

ARB: Androgen Receptor (AR) Binder; E3LB: E3 Ligase Binder.

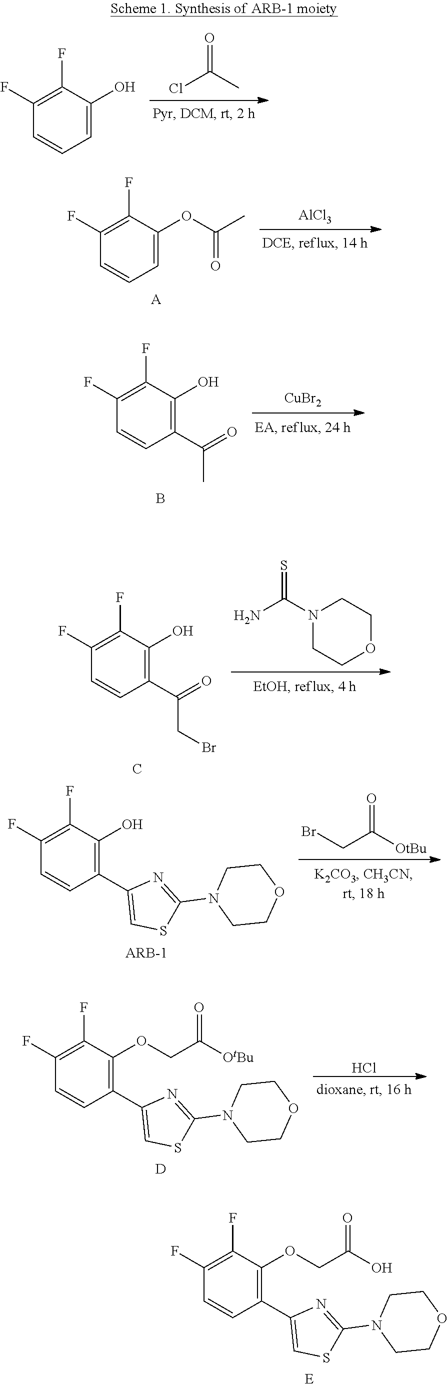

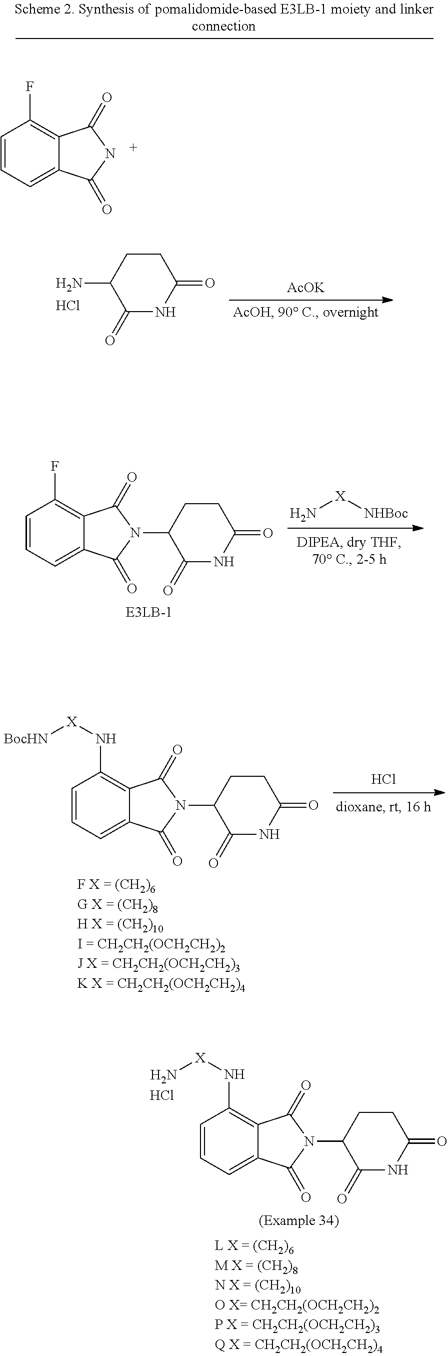

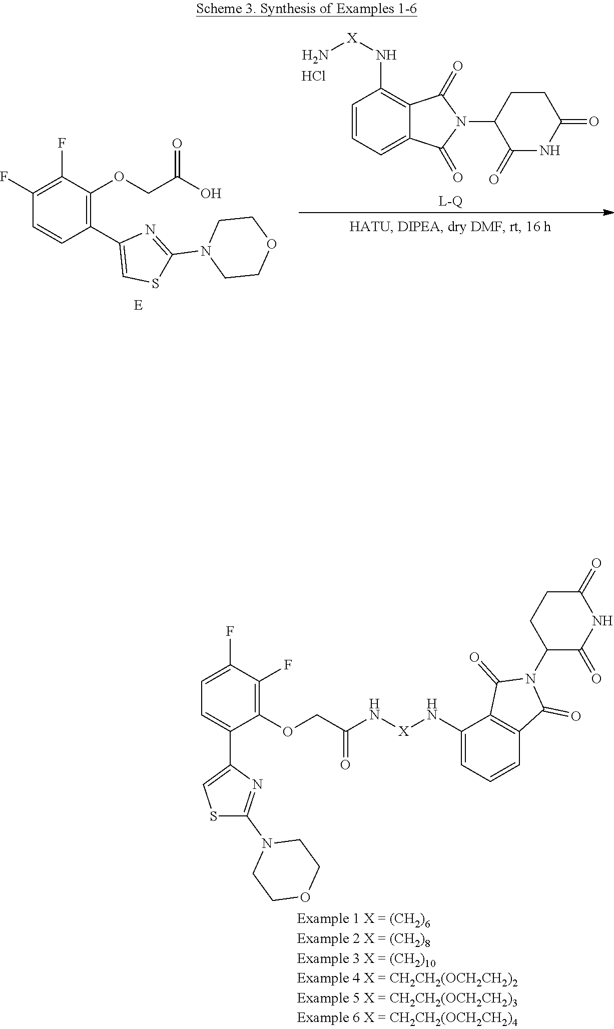

General Procedure III: HATU-Mediated Amidation Reaction.

### 2-(2,3-Difluoro-6-(2-morpholinothiazol-4-yl)phenoxy)-N-(6-((2-(2,6-dioxopiperidin-3-yl)-1,3-dioxoisoindolin-4-yl)amino)hexyl)acetamide (Example 1)

In an oven-dried round-bottom flask, under nitrogen atmosphere, to a stirred solution of 2-(2,3-difluoro-6-(2-morpholinothiazol-4-yl)phenoxy)acetic acid (E) (0.048 g, 0.122 mmol, 1.0 equiv), 4-((6-aminohexyl)amino)-2-(2,6-dioxopiperidin-3-yl)isoindoline-1,3-dione hydrochloride (L) (0.050 g, 0.122 mmol, 1.0 equiv), and DIPEA (0.083 mL, 0.489 mmol, 4.0 equiv) in dry DMF (3.0 mL) was added HATU (0.058 g, 0.153 mmol, 1.25 equiv). Stirring was continued at rt for 16 h. The reaction mixture was diluted with water (30 mL) and extracted with EA (15 mL×3). The reunited organic layers were washed with water (20 mL×3), brine (20 mL×3), dried over anhydrous Na₂SO₄ and then concentrated under reduced pressure to give a crude residue, which was purified by flash column chromatography on SiO₂ (DCM/Acetone/MeOH, 90:10:0 to 89:10:1) affording a yellow solid (0.015 g, 18% yield). ¹H NMR (400 MHz, CDCl₃): δ 8.09 (s, 1H), 7.63-7.56 (m, 1H), 7.56-7.48 (m, 1H), 7.11 (d, J=7.0 Hz, 1H), 7.05 (s, 1H), 7.04-6.96 (m, 1H), 6.93 (s, 1H), 6.90 (d, J=8.5 Hz, 1H), 6.25 (s, 1H), 4.93 (dd, J=5.3, 11.9 Hz, 1H), 4.60 (s, 2H), 3.89-3.78 (m, 4H), 3.64-3.51 (m, 4H), 3.36 (q, J=6.8 Hz, 2H), 3.33-3.26 (m, 2H), 2.97-2.68 (m, 3H), 2.18-2.11 (m, 1H), 1.79-1.35 (m, 8H); ¹³C NMR (101 MHz, CDCl₃): δ 170.87, 170.74, 169.50, 168.25, 167.92, 167.59, 150.76 (dd, J=11.6, 251.4 Hz), 146.96, 146.37-146.09 (m), 144.62 (dd, J=1.5, 9.1 Hz), 144.15 (dd, J=14.0, 247.3 Hz), 136.15, 132.48, 125.04, 124.53 (dd, J=4.0, 7.8 Hz), 116.63, 112.43 (d, J=17.2 Hz), 111.46, 109.90, 105.96, 72.42 (d, J=4.9 Hz), 66.11 (2C), 48.87, 48.62 (2C), 42.52, 38.95, 31.41, 29.42, 29.13, 26.62, 26.55, 22.82. HRMS (ESI) m/z [M+H]+ calcd for C₃₄H₃₆F₂N₆O₇S 711.2407, found 711.2412.

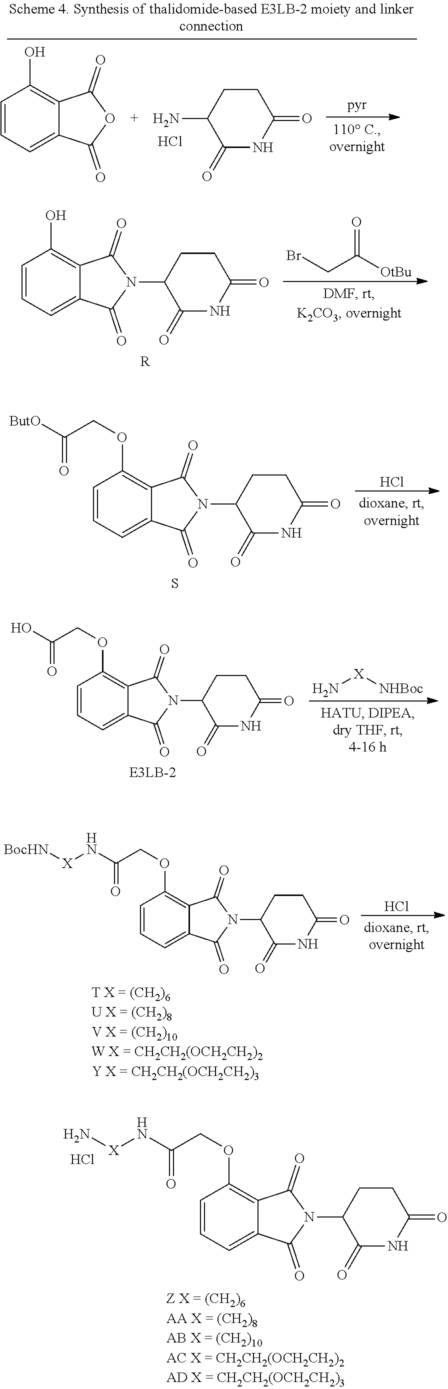

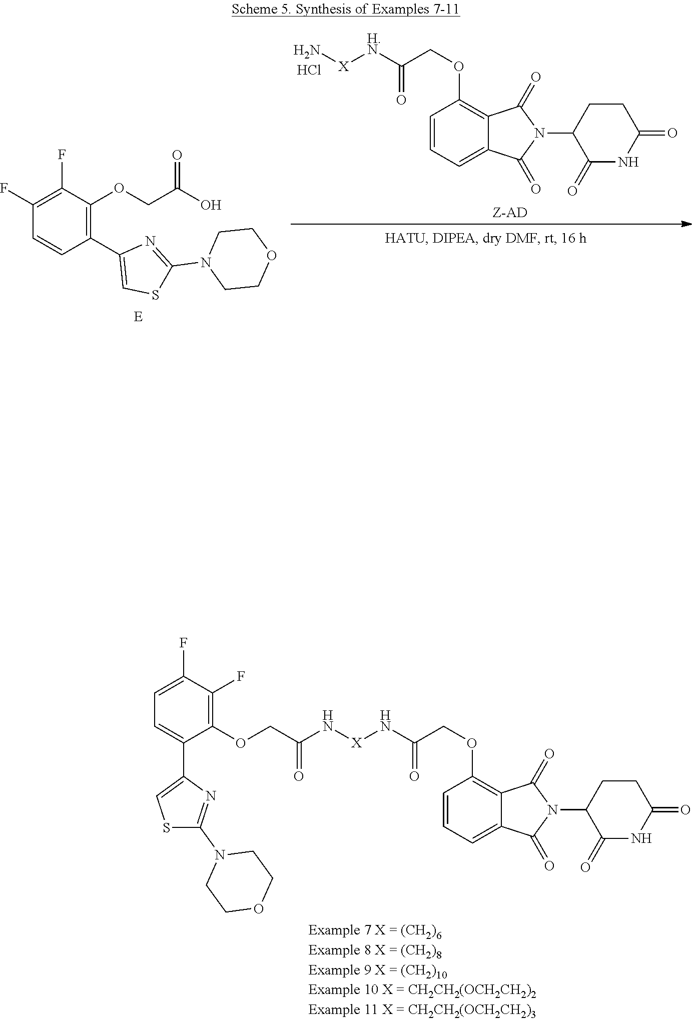

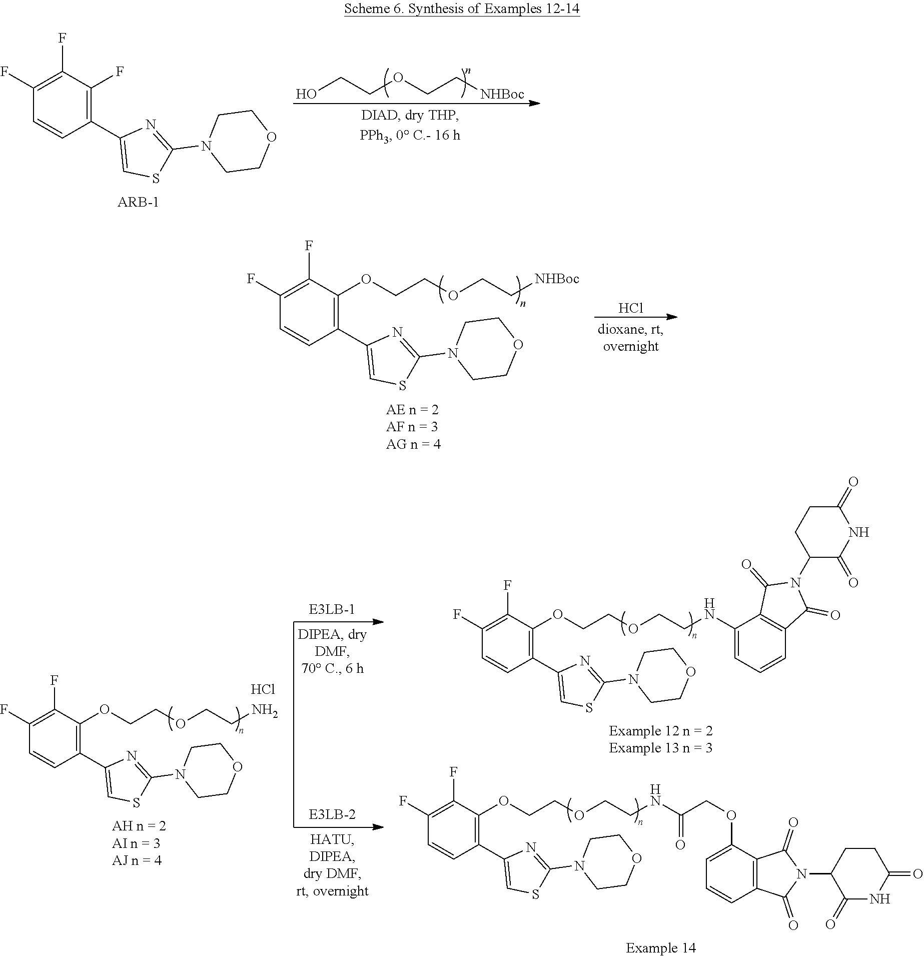

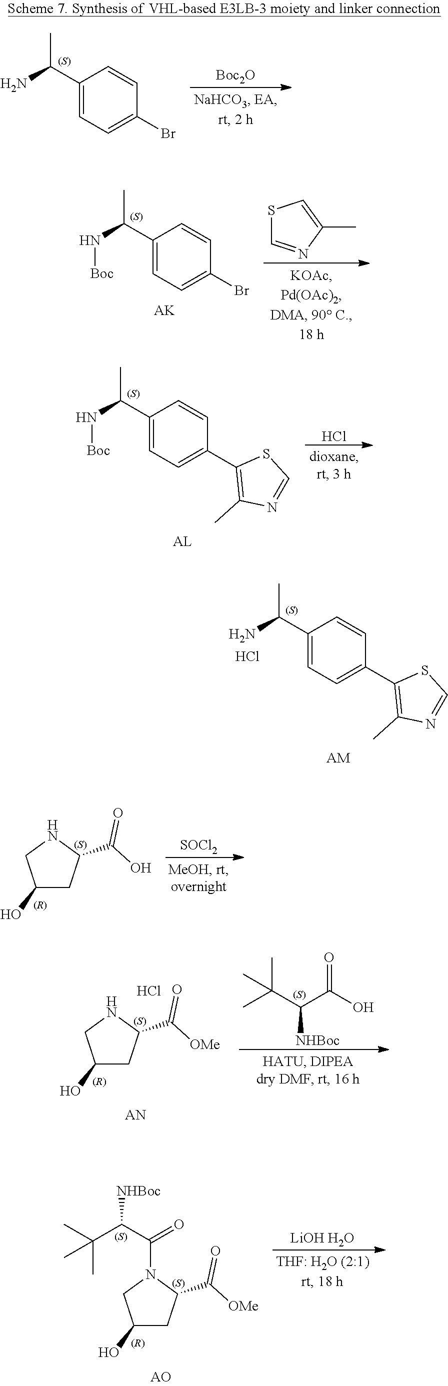

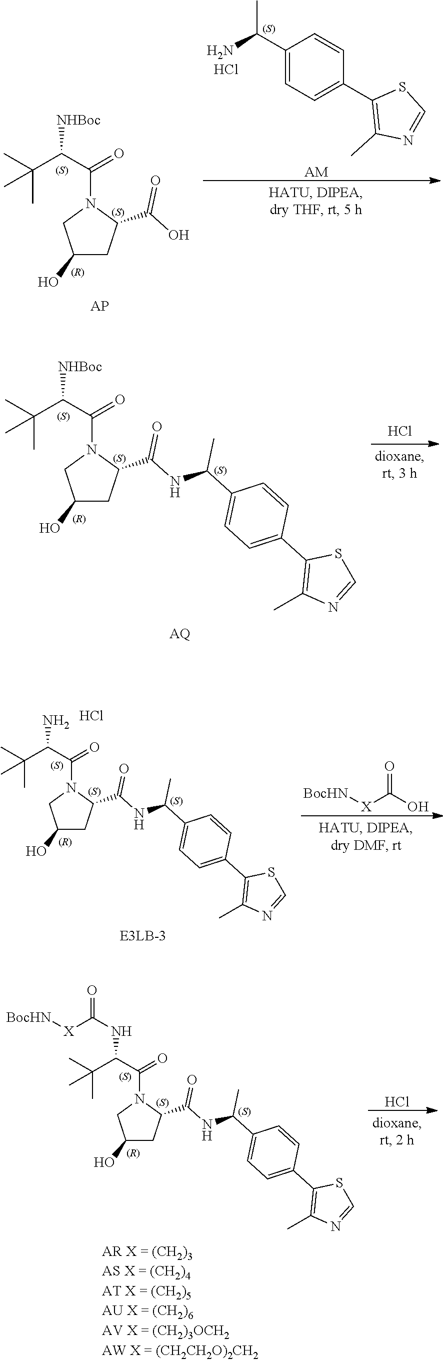

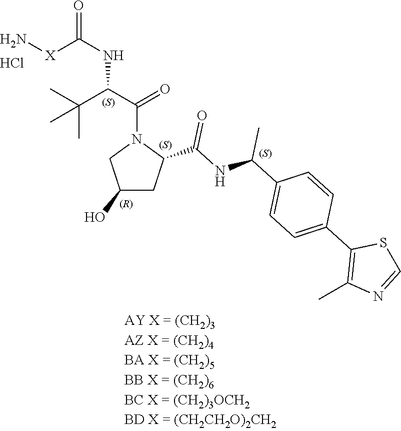

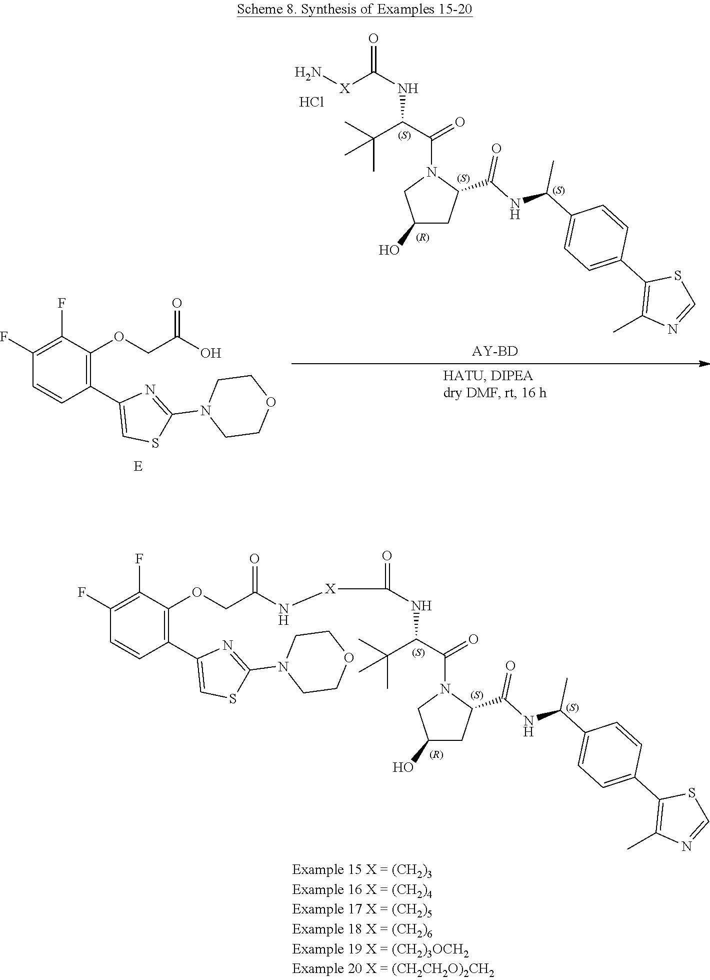

### (2S,4R)-1-((S)-2-(5-(2-(2,3-Difluoro-6-(2-morpholinothiazol-4yl)phenoxy)acetamido)pentanamido)-3,3-dimethylbutanoyl)-4-hydroxy-N—((S)-1-(4-(4-methylthiazol-5-yl)phenyl)ethyl)pyrrolidine-2-carboxamide (Example 16)

General Procedure III (4 hours) was followed by using (2-(2,3-difluoro-6-(2-morpholinothiazol-4-yl)phenoxy)acetic acid (E) (0.67 g, 1.71 mmol), (2S,4R)-1-((S)-2-(5-aminopentanamido)-3,3-dimethylbutanoyl)-4-hydroxy-N—((S)-1-(4-(4-methylthiazol-5-yl)phenyl)ethyl)pyrrolidine-2 carboxamide hydrochloride (AZ) (0.99 g, 1.71 mmol), DIPEA (1.19 mL, 6.82 mmol), and HATU (0.81 g, 2.13 mmol) in dry DMF (7.5 mL) to afford the titled compound as white solid (0.604 g, 40% yield) following purification by flash column chromatography on SiO₂ (DCM/Acetone/MeOH, 60:37:3). ¹H NMR (400 MHz, CDCl₃): δ 8.70 (s, 1H), 7.60 (ddd, J=8.5, 6.0, 2.1 Hz, 1H), 7.48-7.34 (m, 5H), 7.12 (t, J=5.6 Hz, 1H), 7.05-6.92 (m, 2H), 6.32 (d, J=8.6 Hz, 1H), 5.15-5.05 (m, 1H), 4.75 (t, J=8.0 Hz, 1H), 4.66-4.53 (m, 3H), 4.49 (bs, 1H), 4.11 (d, J=11.4 Hz, 1H), 3.90-3.80 (m, 4H), 3.59 (dd, J=11.4, 3.5 Hz, 1H), 3.55-3.46 (m, 4H), 3.45-3.17 (m, 3H), 2.61-2.50 (m, 4H), 2.39-2.21 (m, 2H), 2.07 (dd, J=13.6, 8.3 Hz, 1H), 1.83-1.52 (m, 4H), 1.48 (d, J=6.9 Hz, 3H), 1.06 (s, 9H); ¹³C NMR (101 MHz, CDCl₃): δ 173.34, 172.25, 170.85, 169.54, 168.31, 150.64 (dd, J=251.3, 11.3 Hz), 150.26, 148.50, 146.40 (d, J=1.7 Hz), 144.69-144.42 (m), 144.19 (dd, J=247.5, 14.0 Hz), 143.11, 131.58, 130.88, 129.56 (2C), 126.41 (2C), 125.32 (d, J=3.4 Hz), 124.48 (dd, J=7.7, 3.9 Hz), 112.49 (d, J=17.1 Hz), 106.25, 72.29 (d, J=5.0 Hz), 70.03, 66.13 (2C), 58.24, 57.67, 56.72, 48.86, 48.55 (2C), 38.35, 35.47, 35.35, 34.92, 28.73, 26.51 (3C), 22.40, 22.24, 16.10. HRMS (ESI) m/z [M+H]+ calcd for C₄₃H₅₃F₂N₇O₇S₂ 882.34887, found 882.3458.

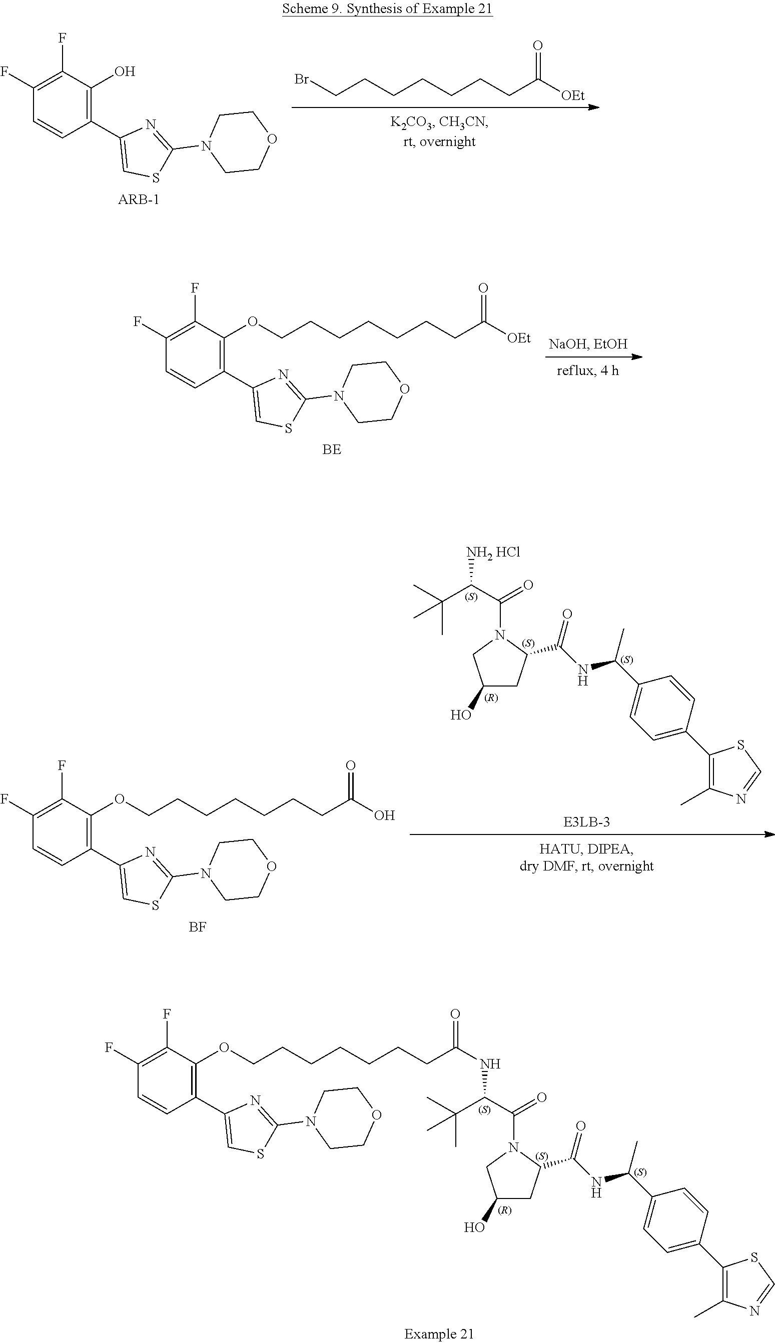

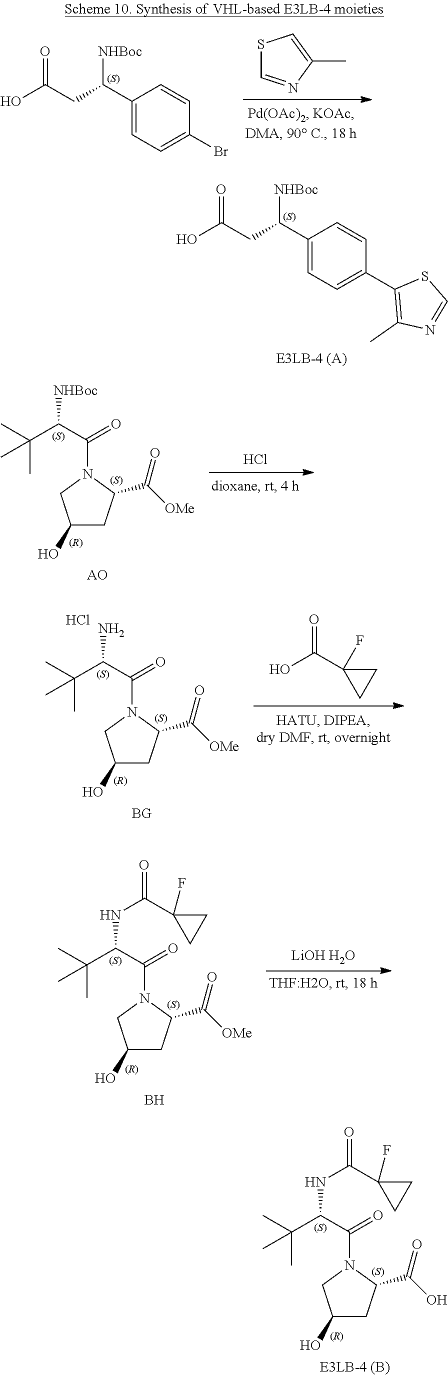

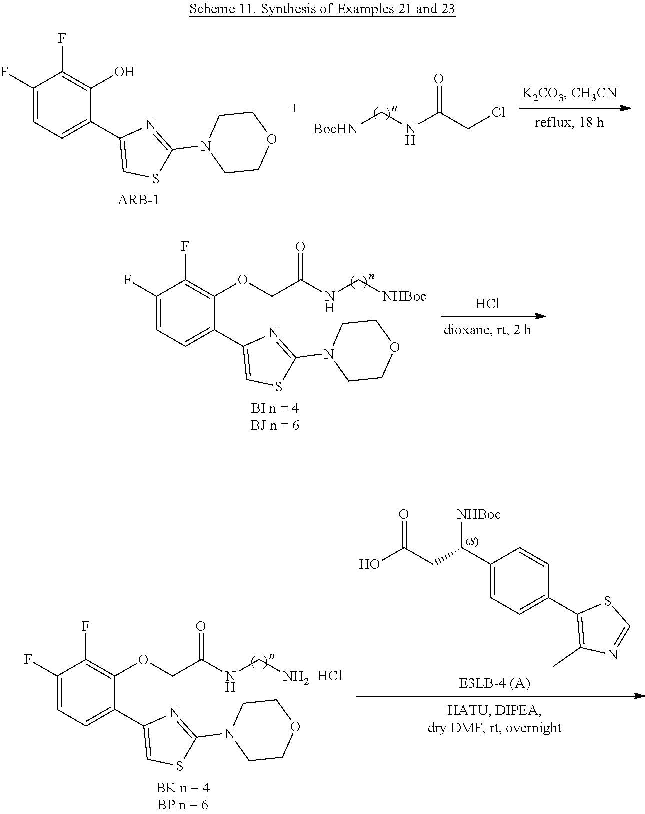

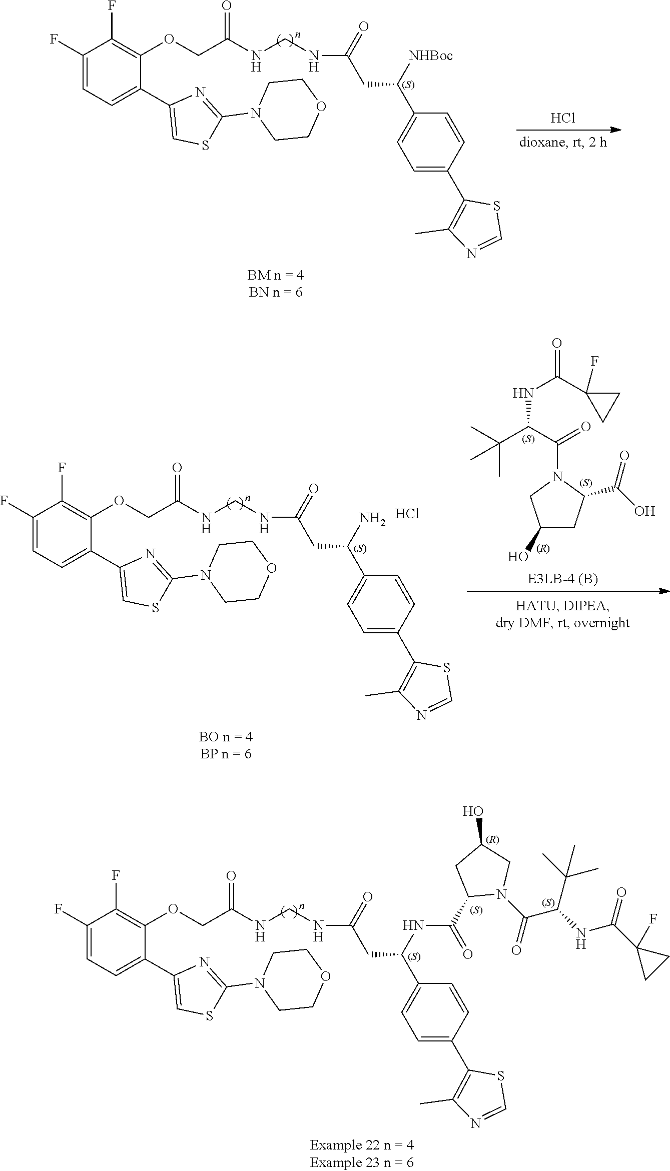

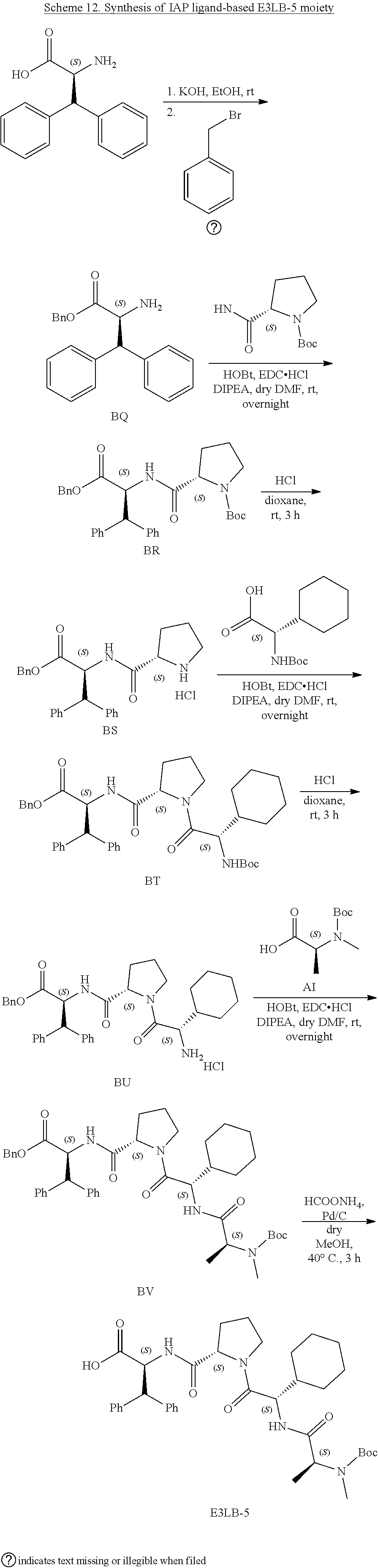

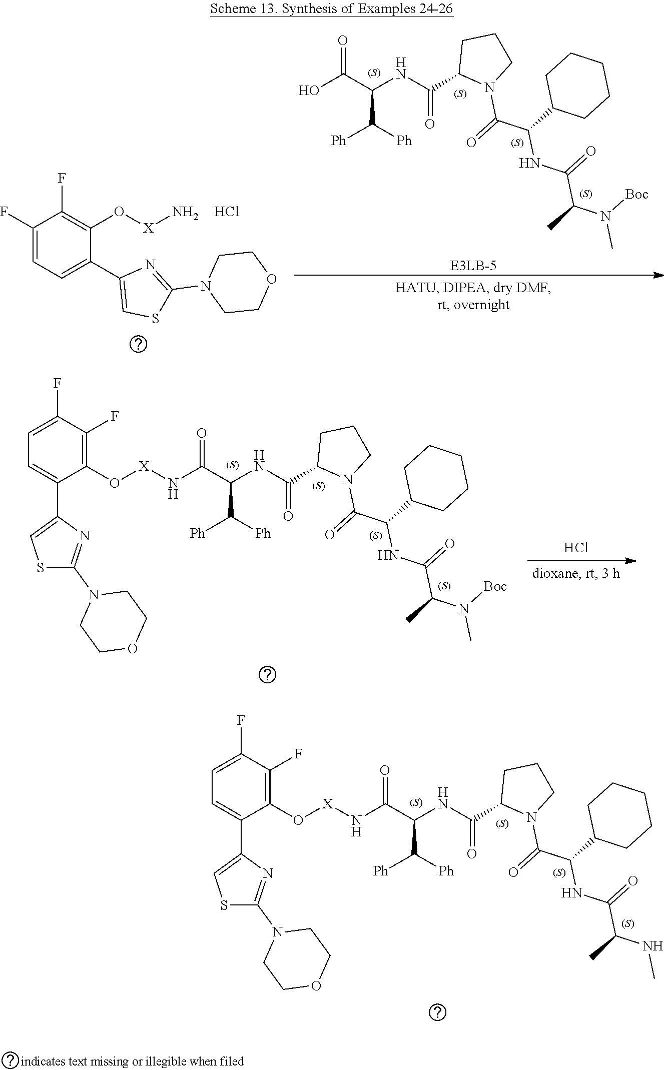

### (S)-1-((S)-2-Cyclohexyl-2-((S)-2-(methylamino)propanamido)acetyl)-N—((S)-1-((2-(2-(2-(2,3-difluoro-6-(2-morpholinothiazol-4-yl)phenoxy)ethoxy)ethoxy)ethyl)amino)-1-oxo-3,3-diphenylpropan-2-yl)pyrrolidine-2-carboxamide (Example 24)

To the solution of tert-butyl ((S)-1-(((S)-1-cyclohexyl-2-((S)-2-(((S)-1-((2-(2-(2-(2,3-difluoro-6-(2-morpholinothiazol-4-yl)phenoxy)ethoxy)ethoxy)ethyl)amino)-1-oxo-3,3-diphenylpropan-2-yl)carbamoyl)pyrrolidin-1-yl)-2-oxoethyl)amino)-1-oxopropan-2-yl)(methyl)carbamate (BW) (0.060 g, 0.056 mmol) in dry DCM (0.5 mL) was added a solution of 4N HCl in dioxane (0.5 mL) and the mixture was stirred at rt for 4 h. The solvent was evaporated to dryness and the residue was diluted with saturated solution of NaHCO₃ (10 mL) and extracted with EA (6 mL×3). The reunited organic phases were washed with brine (10 mL), dried over Na₂SO₄, and evaporated under reduced pressure affording a crude residue which was purified by flash column chromatography on SiO₂ (DCM/MeOH, 95:5 to 94:6) yielding the titled compound (0.041 g, 75% yield) as white solid. HRMS (ESI) m/z [M+Na]+ calcd for C₅₁H₆₅F₂N₇O₈S 996.44756, found 996.44769.

### (S)-1-((S)-2-Cyclohexyl-2-((S)-2-(methylamino)propanamido)acetyl)-N—((S)-1-((4-(2-(2,3-difluoro-6-(2-morpholinothiazol-4-yl)phenoxy)acetamido)butyl)amino)-1-oxo-3,3-diphenylpropan-2-yl)pyrrolidine-2-carboxamide (Example 25)

To the solution of tert-butyl ((S)-1-(((S)-1-cyclohexyl-2-((S)-2-(((S)-1-((4-(2-(2,3-difluoro-6-(2-morpholinothiazol-4-yl)phenoxy)acetamido)butyl)amino)-1-oxo-3,3-diphenylpropan-2-yl)carbamoyl)pyrrolidin-1-yl)-2-oxoethyl)amino)-1-oxopropan-2-yl)(methyl)carbamate (BY) (0.035 g, 0.033 mmol) in dry DCM (0.3 mL) was added a solution of 4N HCl in dioxane (0.3 mL) and the mixture was stirred at rt for 4 h. The solvent was evaporated to dryness and the residue was diluted with saturated solution of NaHCO₃ (10 mL) and extracted with EA (5 mL×3). The reunited organic phases were washed with brine (10 mL), dried over Na₂SO₄, and evaporated under reduced pressure affording a crude residue which was purified by flash column chromatography on SiO₂ (DCM/MeOH, 93:7 to 9:1) yielding the titled compound (0.010 g, 33% yield) as white solid. HRMS (ESI) m/z [M+Na]+ calcd for C₅₁H₆₄F₂N₈O₇S 993.44789, found 993.44843.

### (S)-1-((S)-2-Cyclohexyl-2-((S)-2-(methylamino)propanamido)acetyl)-N—((S)-1-((6-(2-(2,3-difluoro-6-(2-morpholinothiazol-4-yl)phenoxy)acetamido)hexyl)amino)-1-oxo-3,3-diphenylpropan-2-yl)pyrrolidine-2-carboxamide (Example 26)

To the solution of tert-butyl ((S)-1-(((S)-1-cyclohexyl-2-((S)-2-(((S)-1-((6-(2-(2,3-difluoro-6-(2-morpholinothiazol-4-yl)phenoxy)acetamido)hexyl)amino)-1-oxo-3,3-diphenylpropan-2-yl)carbamoyl)pyrrolidin-1-yl)-2-oxoethyl)amino)-1-oxopropan-2-yl)(methyl)carbamate (BZ) (0.030 g, 0.027 mmol) in dry DCM (0.3 mL) was added a solution of 4N HCl in dioxane (0.3 mL) and the mixture was stirred at rt for 4 h. The solvent was evaporated to dryness and the residue was diluted with saturated solution of NaHCO₃ (10 mL) and extracted with EA (5 mL×3). The reunited organic phases were washed with brine (10 mL), dried over Na₂SO₄, and evaporated under reduced pressure affording a crude residue which was purified by flash column chromatography on SiO₂ (DCM/MeOH, 95:5 to 93:7) yielding the titled compound (0.017 g, 63% yield) as white solid. HRMS (ESI) m/z [M+H]+ calcd for C₅₃H₆₈F₂N₈O₇S 999.49725, found 999.49979.

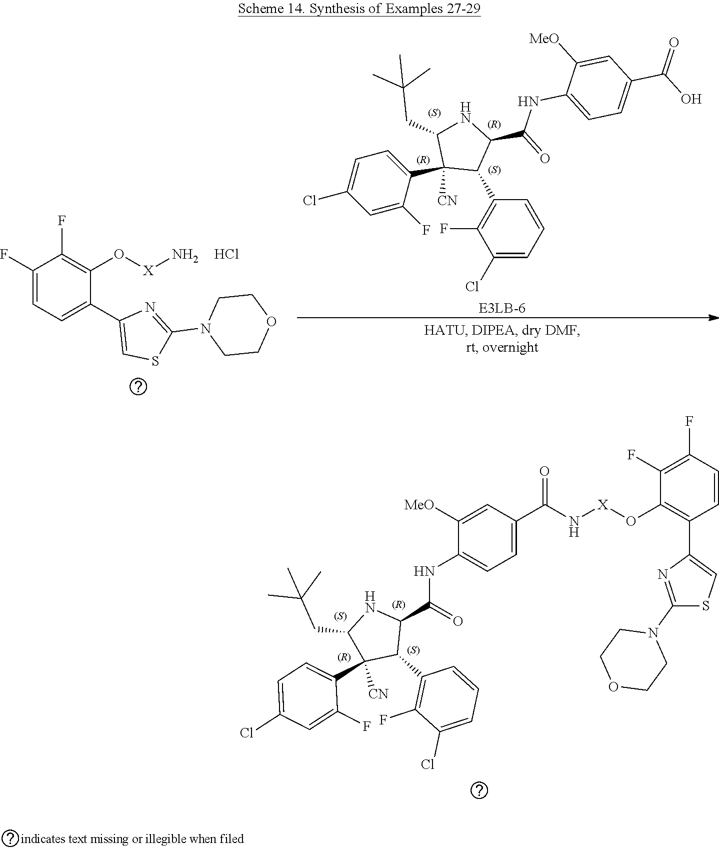

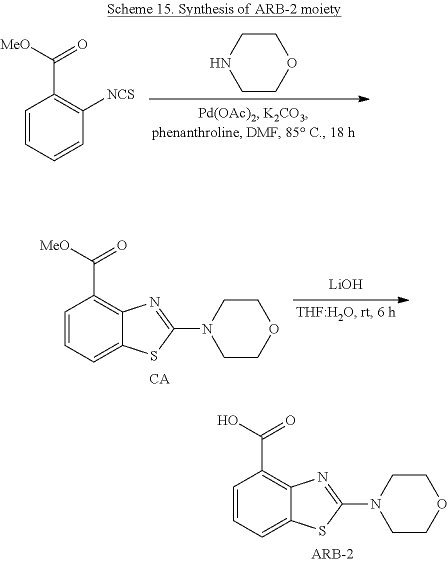

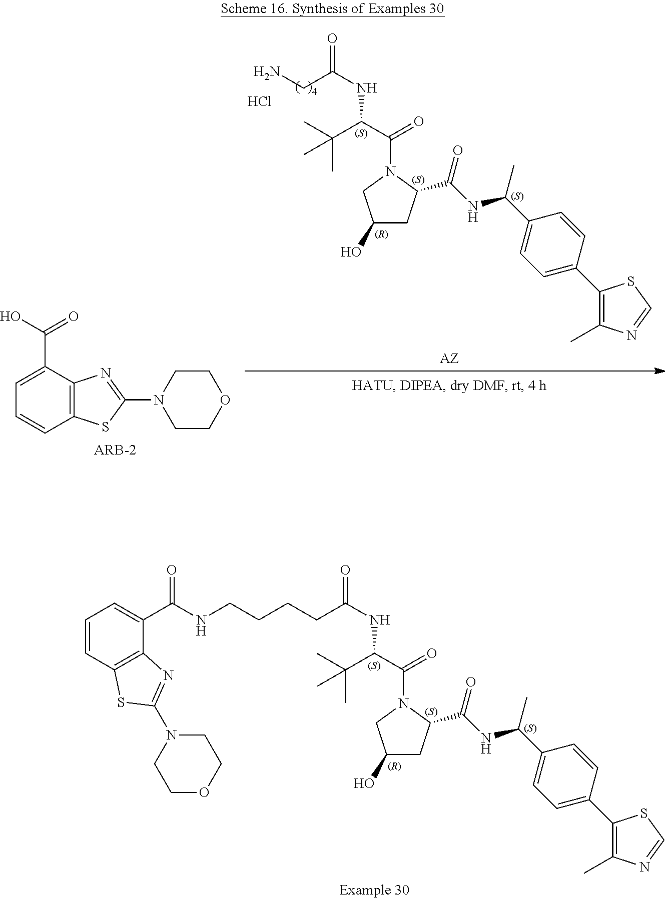

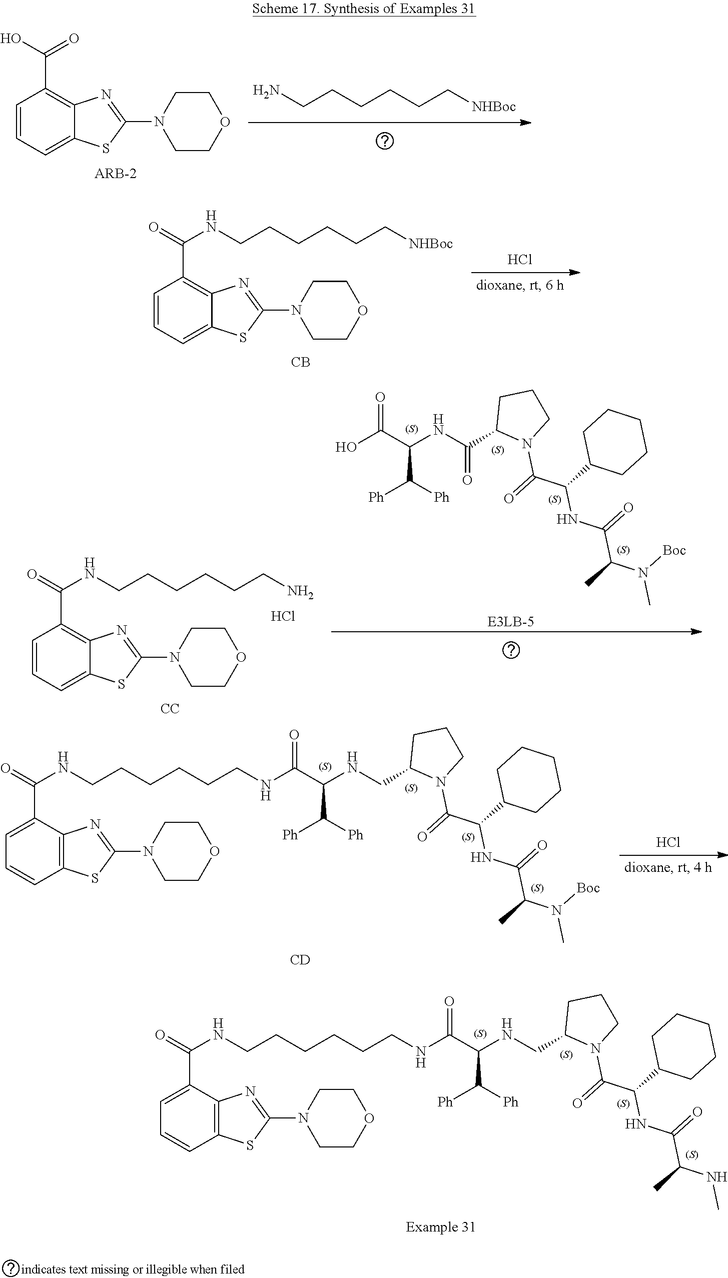

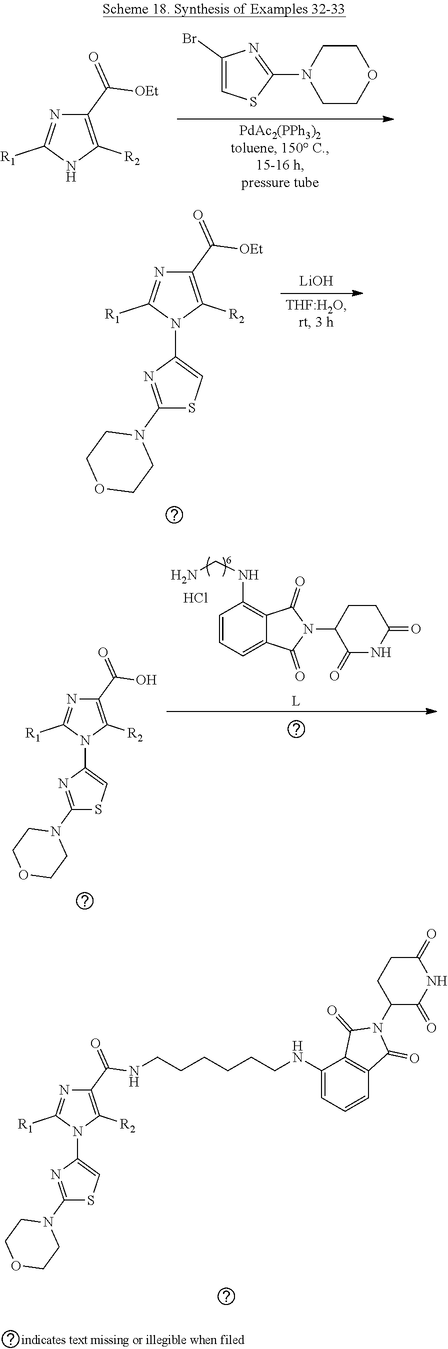

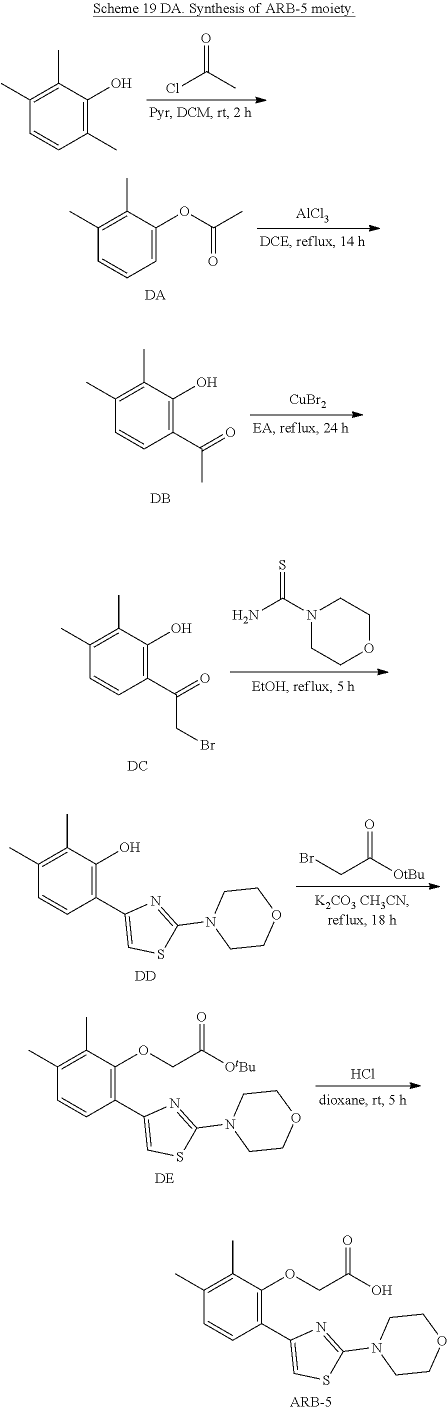

### 2,3-Dimethylphenyl Acetate (DA)

The titled compound can be prepared according to the process described by Singh, V. et al. Tetrahedron Lett. 2015, 56, 1982-1985.

Acetyl chloride (commercially available from, for example, Fluorochem) (0.582 mL, 8.185 mmol) was slowly added at rt to a stirred solution of 2,3-dimethylphenol (commercially available from, for example, Fluorochem) (1.0 g, 8.185 mmol) and pyridine (0.728 mL, 9.004 mmol) in dry DCM (6.0 mL). After 2 h, the mixture was diluted with 2N HCl (20 mL) and the aqueous layer was separated and extracted with DCM (10 mL×4). The reunited organic phases were washed with brine (20 mL×2), dried over anhydrous Na₂SO₄, and concentrated under reduced pressure to afford the titled compound (1.15 g, 86% yield) as a colorless oil. ¹H NMR (400 MHz, CDCl₃) δ 7.11 (t, J=7.7 Hz, 1H), 7.05 (d, J=7.2 Hz, 1H), 6.86 (d, J=7.9 Hz, 1H), 2.33 (s, 3H), 2.30 (s, 3H), 2.08 (s, 3H).

### 1-(2-Hydroxy-3,4-dimethylphenyl)ethan-1-one (DB)

The titled compound can be prepared according to the process described by Nolan K. A. et al. J. Med. Chem. 2009, 52, 7142-7156.

AlCl₃ (0.934 g, 7.003 mmol) was added under nitrogen at 0° C. in small portions to a stirred solution of 2,3-dimethylphenyl acetate (DA) (1.15 g, 7.003 mmol) in DCE (4.0 mL). After addition was completed, the mixture was refluxed for 14 h. After cooling at rt, the solvent was evaporated and the residue diluted with DCM (20 mL). 2N HCl (10 mL) was added dropwise, and the mixture was stirred for 20 min. Organic phase was separated, the water extracted with DCM (10 mL×3), and the reunited organic phases were dried over anhydrous Na₂SO₄ and concentered under reduced pressure to give a brownish oil which was purified by flash column chromatography on SiO₂ (PE/EA, 99:1 to 80:20) affording the titled compound (0.42 g, 36% yield) as a colorless oil. ¹H NMR (400 MHz, CDCl₃) δ 12.68 (s, 1H), 7.49 (d, J=8.2 Hz, 1H), 6.71 (d, J=8.2 Hz, 1H), 2.60 (s, 3H), 2.31 (s, 3H), 2.17 (s, 3H).

### 2,3-Dimethyl-6-(2-morpholinothiazol-4-yl)phenol (DD)

A solution of 1-(2-hydroxy-3,4-dimethylphenyl)ethan-1-one (DB) (0.40 g, 2.436 mmol) in EA (5.0 mL) was added dropwise at rt to a stirred suspension of CuBr₂ (0.816 g, 3.654 mmol) in EA (10 mL). After 24 h of reflux, the mixture was allowed to cool at rt, filtered over Celite, and the filtrate evaporated to dryness. The crude residue was purified by flash column chromatography on SiO₂ (PE/EA, 99:1) to give 2-bromo-1-(2-hydroxy-3,4-dimethylphenyl)ethan-1-one (DC) (0.464 g, 78% yield) as yellow solid, which was used directly in the successive step.

Thus, morpholine-4-carbothioamide (commercially available from, for example, Fluorochem) (0.273 g, 1.872 mmol) was added in small portions to a stirred solution of 2-bromo-1-(2-hydroxy-3,4-dimethylphenyl)ethan-1-one (DC) (0.455 g, 1.872 mmol) in absolute EtOH (6 mL) at 0° C. When the addition was completed, the mixture was refluxed for 5 h and then rt overnight. Then, the mixture was evaporated to dryness and NaHCO₃ saturated solution (20 mL) was added to pH 8. The aqueous phase was extracted with EA (10 mL×3), the reunited organic phases were dried over anhydrous Na₂SO₄ and concentered under reduced pressure. The crude residue was purified by flash column chromatography on SiO₂ (PE/EA, 80:20) to give the titled compound (0.220 g, 40% yield) as white solid. ¹H NMR (400 MHz, CDCl₃) δ 11.95 (bs, 1H), 7.29 (d, J=8.0 Hz, 1H), 6.73 (s, 1H), 6.68 (d, J=8.1 Hz, 1H), 3.88-3.79 (m, 4H), 3.57-3.48 (m, 4H), 2.28 (s, 3H), 2.21 (s, 3H).

### Tert-butyl 2-(2,3-dimethyl-6-(2-morpholinothiazol-4-yl)phenoxy)acetate (DE)

Tert-butyl bromoacetate (commercially available from, for example, Sigma-Aldrich) (0.084 mL, 0.568 mmol) was added to a stirred suspension of 2,3-dimethyl-6-(2-morpholinothiazol-4-yl)phenol (DD) (0.150 g, 0.516 mmol) and K₂CO₃ (0.178 g, 1.291 mmol) in ACN (4.0 mL). The suspension was refluxed for 18 h, filtered, and the filtrate evaporated to dryness. Residue was purified by automated flash chromatography on SiO₂ cartridge (PE/EA, 95:5 to 60:40) to afford the titled compound (0.180 g, 86% yield) as a colorless oil. ¹H NMR (400 MHz, CDCl₃) δ 7.75 (d, J=7.9 Hz, 1H), 7.38 (s, 1H), 6.96 (d, J=7.9 Hz, 1H), 4.19 (s, 2H), 3.84-3.76 (m, 4H), 3.52-3.46 (m, 4H), 2.25 (s, 6H), 1.49 (s, 9H).

### 2-(2,3-Dimethyl-6-(2-morpholinothiazol-4-yl)phenoxy)acetic acid (ARB-5)

A solution of 4N HCl in dioxane (2.0 mL) was added at 0° C. to tert-butyl 2-(2,3-dimethyl-6-(2-morpholinothiazol-4-yl)phenoxy)acetate (DE) (0.170 g, 0.420 mmol) and the resulting suspension was stirred at rt for 5 h. The solvent was evaporated to dryness and the residue was triturated with DEE. The solids were collected by filtration and dried under vacuo to afford the titled compound (0.130 g, 89% yield) as white solid. ¹H NMR (400 MHz, CDCl₃) δ 7.31 (d, J=7.8 Hz, 1H), 7.03 (d, J=7.9 Hz, 1H), 6.84 (s, 1H), 4.50 (s, 2H), 3.95-3.83 (m, 8H), 2.29 (s, 3H), 2.24 (s, 3H).

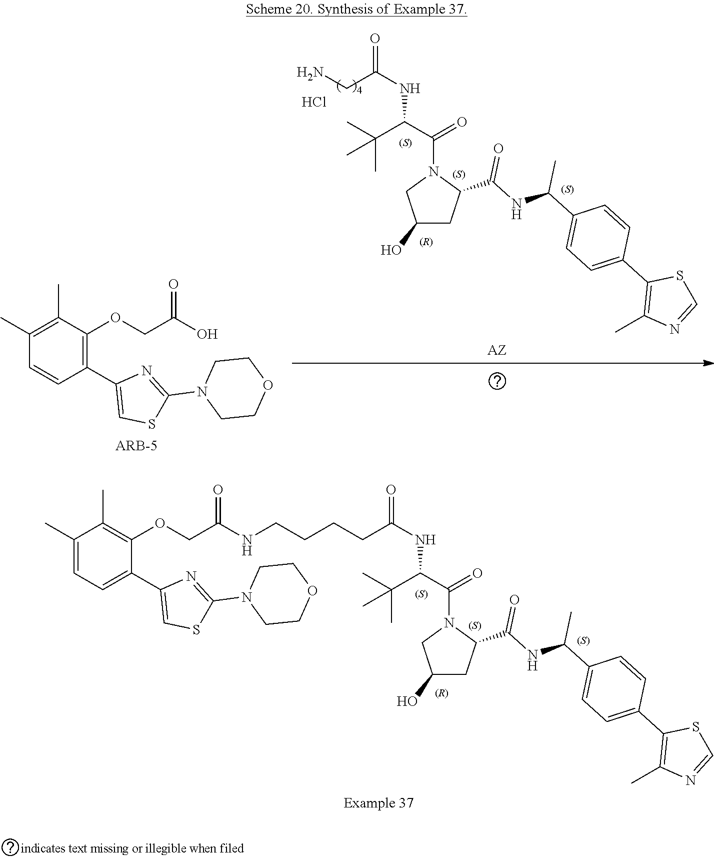

### (2S,4R)-1-((S)-2-(5-(2-(2,3-dimethyl-6-(2-morpholinothiazol-4-yl)phenoxy)acetamido)pentanamido)-3,3-dimethylbutanoyl)-4-hydroxy-N—((S)-1-(4-(4-methylthiazol-5-yl)phenyl)ethyl)pyrrolidine-2-carboxamide (Example 37)

In an oven-dried round-bottom flask, under nitrogen atmosphere, to a stirred solution of 2-(2,3-dimethyl-6-(2-morpholinothiazol-4-yl)phenoxy)acetic acid (ARB-5) (0.027 g, 0.077 mmol), (2S,4R)-1-((S)-2-(5-Aminopentanamido)-3,3-dimethylbutanoyl)-4-hydroxy-N—((S)-1-(4-(4-methylthiazol-5-yl)phenyl)ethyl)pyrrolidine-2-carboxamide hydrochloride (AZ) (0.045 g, 0.077 mmol) and DIPEA (0.054 mL, 0.310 mmol) in dry DMF (1.0 mL) was added HATU (0.036 g, 0.096 mmol). Stirring was continued at rt for 5 h. The reaction mixture was diluted with water (30 mL) and extracted with EA (10 mL×3). The reunited organic layers were washed with water (10 mL×3), brine (10 mL×3), dried over anhydrous Na₂SO₄ and then concentrated under reduced pressure to give a crude residue, which was purified by automated flash chromatography on SiO₂ cartridge (DCM/MeOH, 99:1 to 90:10) affording the titled compound (18.6 mg, 27% yield) as white solid. ¹H NMR (400 MHz, CDCl₃) δ 8.66 (s, 1H), 7.48 (d, J=7.9 Hz, 1H), 7.43 (d, J=7.8 Hz, 1H), 7.40-7.31 (m, 4H), 7.11 (t, J=6.0 Hz, 1H), 6.97 (d, J=7.9 Hz, 1H), 6.82 (s, 1H), 6.45 (d, J=8.5 Hz, 1H), 5.10-5.01 (m, 1H), 4.69 (t, J=8.0 Hz, 1H), 4.56 (d, J=8.8 Hz, 1H), 4.42 (s, 1H), 4.29-4.20 (m, 2H), 4.05 (d, J=11.3 Hz, 1H), 3.84-3.78 (m, 4H), 3.55 (dd, J=11.2, 3.5 Hz, 1H), 3.49-3.44 (m, 4H), 3.37-3.23 (m, 2H), 2.52 (s, 3H), 2.46-2.29 (m, 2H), 2.28-2.21 (m, 4H), 2.18 (s, 3H), 2.03-1.97 (m, 1H), 1.74-1.61 (m, 2H), 1.61-1.51 (m, 2H), 1.44 (d, J=6.9 Hz, 3H), 1.04 (s, 9H). HRMS (ESI) m/z [M+Na]+ calcd for C₄₅H₅₉N₇O₇S₂ 896.38096, found 896.38278.

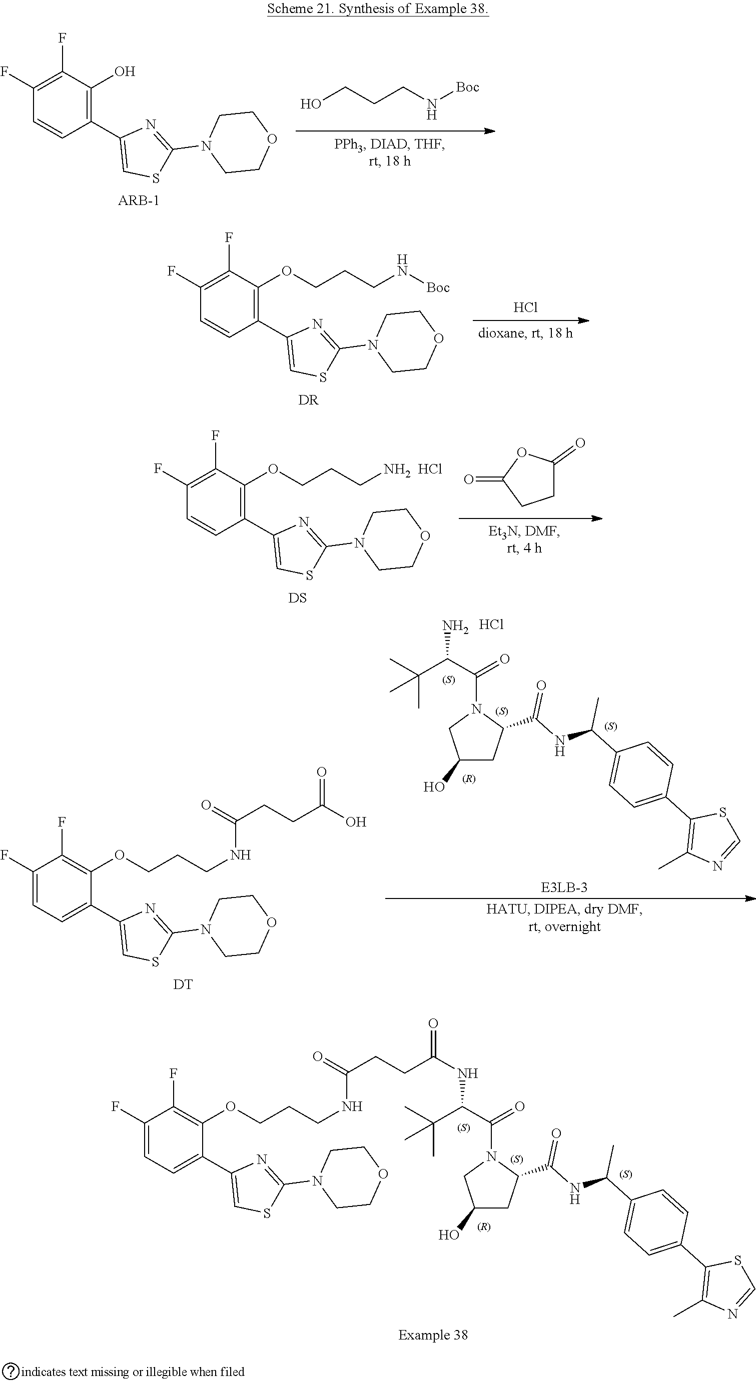

### Tert-butyl (3-(2,3-difluoro-6-(2-morpholinothiazol-4-yl)phenoxy)propyl) carbamate (DR)

DIAD (0.109 mL, 0.553 mmol) was slowly added to a stirred ice-cooled solution of 2,3-difluoro-6-(2-morpholinothiazol-4-yl)phenol (ARB-1) (0.150 g, 0.502 mmol), tert-butyl (3-hydroxypropyl)carbamate (0.097 g, 0.553 mmol), and PPh₃ (0.134 g, 0.553 mmol) in dry THF (2.0 mL). The solution was stirred at 0° C. for 30 min, then at rt for 18 h. The reaction mixture was evaporated to dryness to afford a crude residue which was purified by flash column chromatography on SiO₂ (DCM/MeOH, 99:1) to give the titled compound (0.135 g, 59% yield) as colorless oil. ¹H NMR (400 MHz, CDCl₃) δ 7.75 (ddd, J=8.8, 6.2, 2.3 Hz, 1H), 7.17 (s, 1H), 6.97-6.86 (m, 1H), 4.81 (bs, 1H), 4.11 (t, J=5.9 Hz, 2H), 3.88-3.77 (m, 4H), 3.56-3.44 (m, 4H), 3.38-3.26 (m, 2H), 2.01-1.89 (m, 2H), 1.43 (s, 9H).

### 3-(2,3-Difluoro-6-(2-morpholinothiazol-4-yl)phenoxy)propan-1-amine hydrochloride (DS)

A solution of 4N HCl in dioxane (1.5 mL) was added at 0° C. to a solution of tert-butyl (3-(2,3-difluoro-6-(2-morpholinothiazol-4-yl)phenoxy)propyl)carbamate (DR) (0.130 g, 0.285 mmol) in dry DCM (1.0 mL) and the resulting solution was stirred at rt overnight. The solvent was evaporated to dryness and the residue was triturated with DEE, collected by filtration, and dried under vacuo to afford the titled compound (0.094 g, 84% yield) as white solid. ¹H NMR (400 MHz, CD₃OD) δ 7.49-7.40 (m, 1H), 7.20 (s, 1H), 7.19-7.12 (m, 1H), 4.25 (t, J=6.0 Hz, 2H), 3.94-3.85 (m, 4H), 3.76-3.67 (m, 4H), 3.15-3.06 (m, 2H), 2.17-2.06 (m, 2H).

### 4-((3-(2,3-Difluoro-6-(2-morpholinothiazol-4-yl)phenoxy)propyl)amino)-4-oxobutanoic acid (DT)

Under nitrogen atmosphere, to a stirred solution of 3-(2,3-difluoro-6-(2-morpholinothiazol-4-yl)phenoxy)propan-1-amine hydrochloride (DS) (0.088 g, 0.224 mmol) and Et₃N (0.094 mL, 0.672 mmol) in dry DMF (1.0 mL) was added succinic anhydride (0.027 mg, 0.269 mmol) and the reaction mixture was stirred a rt for 4 h. The reaction mixture was poured in ice-water (30 mL) and extracted with EA (10 mL×3). The reunited organic layers were washed with water (10 mL×3), brine (10 mL×3), dried over anhydrous Na₂SO₄ and then concentrated under reduced pressure to give affording the titled compound (82.0 mg, 80% yield) as white solid. ¹H NMR (400 MHz, CDCl₃) δ 7.69 (ddd, J=8.7, 6.2, 2.2 Hz, 1H), 7.11 (s, 1H), 6.96-6.86 (m, 1H), 6.41 (t, J=5.6 Hz, 1H), 4.15-4.06 (m, 2H), 3.88-3.77 (m, 4H), 3.53-3.41 (m, 6H), 2.69-2.61 (m, 2H), 2.44 (t, J=6.7 Hz, 2H), 2.01-1.91 (m, 2H).

### N¹-(3-(2,3-Difluoro-6-(2-morpholinothiazol-4-yl)phenoxy)propyl)-N—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)succinamide (Example 38)

In an oven-dried round-bottom flask, under nitrogen atmosphere, to a stirred solution of 4-((3-(2,3-difluoro-6-(2-morpholinothiazol-4-yl)phenoxy)propyl)amino)-4-oxobutanoic acid (DT) (0.077 g, 0.169 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (E3LB-3) (0.082 g, 0.169 mmol) and DIPEA (0.120 mL, 0.676 mmol) in dry DMF (1.0 mL) was added HATU (0.081 g, 0.213 mmol). Stirring was continued at rt overnight. The reaction mixture was diluted with water (30 mL) and extracted with EA (10 mL×3). The reunited organic layers were washed with water (10 mL×3), brine (10 mL×3), dried over anhydrous Na₂SO₄ and then concentrated under reduced pressure to give a crude residue, which was purified by flash column chromatography on SiO₂ (DCM/Acetone/MeOH, 75:20:5) affording the titled compound (43.0 mg, 29% yield) as white solid. ¹H NMR (400 MHz, CDCl₃) δ 8.72 (s, 1H), 7.73-7.62 (m, 1H), 7.54 (d, J=7.7 Hz, 1H), 7.44-7.33 (m, 4H), 7.07 (s, 1H), 6.98-6.88 (m, 2H), 6.39 (bs, 1H), 5.14-5.02 (m, 1H), 4.74 (t, J=8.0 Hz, 1H), 4.50 (d, J=8.4 Hz, 1H), 4.45 (s, 1H), 4.15 (t, J=5.6 Hz, 2H), 4.03 (d, J=11.4 Hz, 1H), 3.91-3.77 (m, 4H), 3.68-3.48 (m, 5H), 3.48-3.38 (m, 2H), 2.64-2.38 (m, 8H), 2.13-2.02 (m, 1H), 2.01-1.91 (m, 2H), 1.46 (d, J=7.0 Hz, 3H), 1.04 (s, 9H). HRMS (ESI) m/z [M+H]+ calcd for C₄₃H₅₃F₂N₇O₇S₂ 882.34887, found 882.35058.

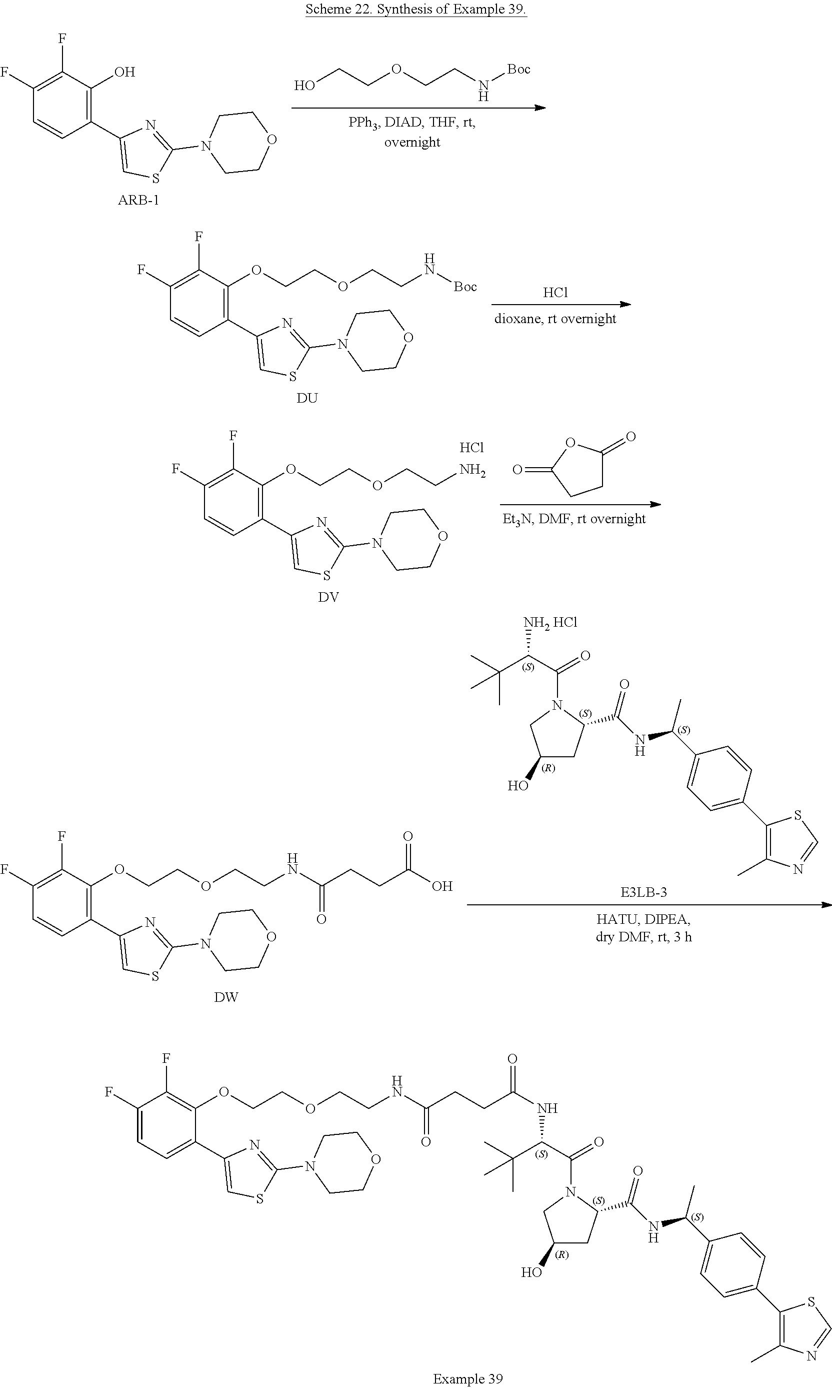

### Tert-butyl (2-(2-(2,3-difluoro-6-(2-morpholinothiazol-4-yl)phenoxy)ethoxy)ethyl) carbamate (DU)

DIAD (0.180 mL, 0.920 mmol) was slowly added to a stirred ice-cooled solution of 2,3-difluoro-6-(2-morpholinothiazol-4-yl)phenol (ARB-1) (0.250 g, 0.838 mmol), tert-butyl (2-(2-hydroxyethoxy)ethyl)carbamate (0.189 g, 0.920 mmol), and PPh₃ (0.223 g, 0.920 mmol) in dry THF (2.0 mL). The solution was stirred at 0° C. for 30 min, then at rt overnight. The reaction mixture was evaporated to dryness to afford a crude residue which was purified by flash column chromatography on SiO₂ (DCM/MeOH, 99:1) to give the titled compound (0.236 g, 58% yield) as colourless oil. ¹H NMR (400 MHz, CDCl₃) δ 7.85 (ddd, J=8.8, 6.2, 2.3 Hz, 1H), 7.44 (s, 1H), 6.98-6.89 (m, 1H), 4.90 (bs, 1H), 4.31-4.23 (m, 2H), 3.88-3.81 (m, 4H), 3.80-3.75 (m, 2H), 3.58-3.51 (m, 6H), 3.37-3.27 (m, 2H), 1.44 (s, 9H).

### 2-(2-(2,3-Difluoro-6-(2-morpholinothiazol-4-yl)phenoxy)ethoxy)ethan-1-amine hydrochloride (DV)

A solution of 4N HCl in dioxane (3.5 mL) was added at 0° C. to a solution of tert-butyl (2-(2-(2,3-difluoro-6-(2-morpholinothiazol-4-yl)phenoxy)ethoxy)ethyl)carbamate (DU) (0.236 g, 0.486 mmol) in dry DCM (1.0 mL) and the resulting solution was stirred at rt overnight. The solvent was evaporated to dryness and the residue was triturated with DEE, collected by filtration, and dried under vacuo to afford the titled compound (0.160 g, 86% yield) as colorless oil. ¹H NMR (400 MHz, CD₃OD) δ 7.83 (ddd, J=8.8, 6.3, 2.3 Hz, 1H), 7.51 (s, 1H), 7.04-6.95 (m, 1H), 4.33-4.24 (m, 2H), 3.84-3.74 (m, 6H), 3.53-3.43 (m, 6H), 2.77 (t, J=5.3 Hz, 2H).

### 4-((2-(2-(2,3-Difluoro-6-(2-morpholinothiazol-4-yl)phenoxy)ethoxy)ethyl)amino)-4-oxobutanoic acid (DW)

Under nitrogen atmosphere, to a stirred solution of 2-(2-(2,3-Difluoro-6-(2-morpholinothiazol-4-yl)phenoxy)ethoxy)ethan-1-amine hydrochloride (DV) (0.096 g, 0.227 mmol) and Et₃N (0.096 mL, 0.681 mmol) in dry DMF (1.0 mL) was added succinic anhydride (0.028 mg, 0.272 mmol) and the reaction mixture was stirred a rt overnight. The reaction mixture was poured in ice-water (30 mL) and extracted with EA (10 mL×3). The reunited organic layers were washed with water (10 mL×3), brine (10 mL×3), dried over anhydrous Na₂SO₄ and then concentrated under reduced pressure to give affording the titled compound (55 mg, 50% yield) as white solid. ¹H NMR (400 MHz, DMSO-d₆) δ 12.05 (bs, 1H), 7.97-7.80 (m, 2H), 7.59 (s, 1H), 7.25-7.15 (m, 1H), 4.32-4.19 (m, 2H), 3.86-3.65 (m, 6H), 3.53-3.37 (m, 6H), 3.25-3.14 (m, 2H), 2.45-2.36 (m, 2H), 2.35-2.26 (m, 2H).

### N¹-(2-(2-(2,3-difluoro-6-(2-morpholinothiazol-4-yl)phenoxy)ethoxy)ethyl)-N⁴—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)succinamide (Example 39)

In an oven-dried round-bottom flask, under nitrogen atmosphere, to a stirred solution of 4-((2-(2-(2,3-difluoro-6-(2-morpholinothiazol-4-yl)phenoxy)ethoxy)ethyl)amino)-4-oxobutanoic acid (DW) (0.051 g, 0.105 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (E3LB-3) (0.050 g, 0.105 mmol) and DIPEA (0.073 mL, 0.420 mmol) in dry DMF (1.0 mL) was added HATU (0.050 g, 0.131 mmol). Stirring was continued at rt 3 h. The reaction mixture was diluted with water (30 mL) and extracted with EA (10 mL×3). The reunited organic layers were washed with water (10 mL×3), brine (10 mL×3), dried over anhydrous Na₂SO₄ and then concentrated under reduced pressure to give a crude residue, which was purified by flash column chromatography on SiO₂ (DCM/Acetone/MeOH, 75:20:5) affording the titled compound (36.0 mg, 37% yield) as white solid. ¹H NMR (400 MHz, CDCl₃) δ 8.77 (s, 1H), 7.82 (ddd, J=8.5, 6.2, 1.9 Hz, 1H), 7.56 (d, J=7.6 Hz, 1H), 7.43-7.33 (m, 5H), 7.03-6.89 (m, 2H), 6.24 (bs, 1H), 5.12-5.02 (m, 1H), 4.74 (t, J=8.0 Hz, 1H), 4.49 (d, J=8.4 Hz, 1H), 4.44 (s, 1H), 4.33-4.24 (m, 2H), 4.05 (d, J=11.4 Hz, 1H), 3.91-3.78 (m, 4H), 3.78-3.70 (m, 2H), 3.69-3.61 (m, 1H), 3.58-3.49 (m, 6H), 3.44-3.37 (m, 2H), 2.59-2.40 (m, 8H), 2.12-2.01 (m, 1H), 1.46 (d, J=6.9 Hz, 3H), 1.04 (s, 9H). HRMS (ESI) m/z [M+H]+ calcd for C₄₄H₅₅F₂N₇O₈S₂ 912.35944, found 912.36178.

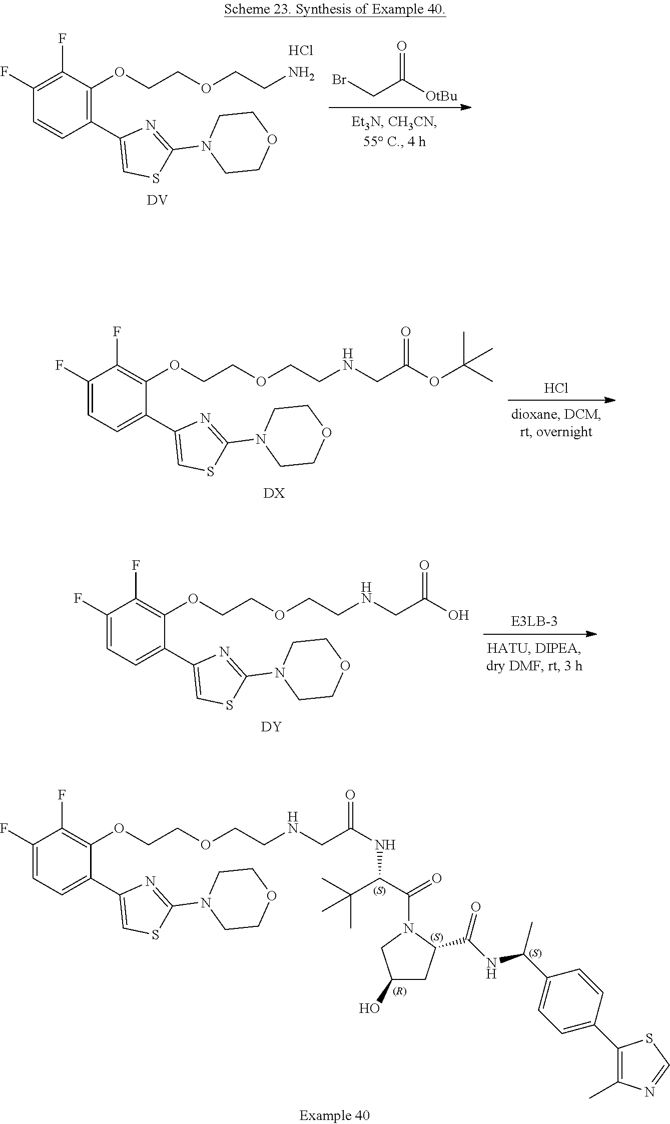

### Tert-butyl (2-(2-(2,3-difluoro-6-(2-morpholinothiazol-4-yl)phenoxy)ethoxy)ethyl) glycinate (DX)

Tert-butyl bromoacetate (0.021 mL, 0.143 mmol) was added to a stirred solution of 2-(2-(2,3-difluoro-6-(2-morpholinothiazol-4-yl)phenoxy)ethoxy)ethan-1-amine hydrochloride (DV) (0.055 g, 0.130 mmol) and Et₃N (0.045 mL, 0.325 mmol) in ACN (2.0 mL). The solution was stirred at 55° C. for 4 h, filtered, and the filtrate evaporated to dryness. Residue was purified by automated flash chromatography on SiO₂ cartridge (DCM/MeOH, 99:1 to 95:5) to afford the titled compound (0.0300 g, 46% yield) as a colorless oil. ¹H NMR (400 MHz, CDCl₃) δ 7.86 (ddd, J=8.7, 6.2, 2.2 Hz, 1H), 7.51 (s, 1H), 6.97-6.86 (m, 1H), 4.33-4.22 (m, 2H), 3.89-3.76 (m, 6H), 3.64 (t, J=5.2 Hz, 2H), 3.54-3.46 (m, 4H), 3.37 (s, 2H), 2.90-2.81 (m, 2H), 1.46 (s, 9H).

### (2-(2-(2,3-Difluoro-6-(2-morpholinothiazol-4-yl)phenoxy)ethoxy)ethyl)glycine (DY)

A solution of 4N HCl in dioxane (0.80 mL) was added at 0° C. to a solution of tert-butyl (2-(2-(2,3-difluoro-6-(2-morpholinothiazol-4-yl)phenoxy)ethoxy)ethyl)glycinate (DX) (0.042 g, 0.084 mmol) in dry DCM (1.0 mL) and the resulting solution was stirred at rt overnight. The solvent was evaporated to dryness and the residue was triturated with DEE to afford the titled compound (0.040 g, 99% yield) as colorless oil. ¹H NMR (400 MHz, CD₃OD) δ 7.70-7.50 (m, 1H), 7.32 (s, 1H), 7.11 (dd, J=16.9, 9.1 Hz, 1H), 4.36 (s, 2H), 3.95 (s, 2H), 3.89-3.76 (m, 8H), 3.68-3.61 (m, 4H).

### (2S,4R)-1-((S)-2-(2-((2-(2-(2,3-difluoro-6-(2-morpholinothiazol-4-yl)phenoxy)ethoxy)ethyl)amino)acetamido)-3,3-dimethylbutanoyl)-4-hydroxy-N—((S)-1-(4-(4-methylthiazol-5-yl)phenyl)ethyl)pyrrolidine-2-carboxamide (Example 40)

In an oven-dried round-bottom flask, under nitrogen atmosphere, to a stirred solution of (2-(2-(2,3-difluoro-6-(2-morpholinothiazol-4-yl)phenoxy)ethoxy)ethyl)glycine (DY) (0.040 g, 0.090 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (E3LB-3) (0.043 g, 0.090 mmol) and DIPEA (0.063 mL, 0.0361 mmol) in dry DMF (1.0 mL) was added HATU (0.042 g, 0.110 mmol). Stirring was continued at rt 3 h. The reaction mixture was diluted with water (30 mL) and extracted with EA (10 mL×3). The reunited organic layers were washed with water (10 mL×3), brine (10 mL×3), dried over anhydrous Na₂SO₄ and then concentrated under reduced pressure to give a crude residue, which was purified by flash column chromatography on SiO₂ (DCM/MeOH, 95:5) affording the titled compound (9.0 mg, 12% yield) as white solid. ¹H NMR (400 MHz, CDCl₃) δ 8.69 (s, 1H), 7.94-7.82 (m, 2H), 7.54 (d, J=7.4 Hz, 1H), 7.47 (s, 1H), 7.44-7.32 (m, 4H), 7.00-6.90 (m, 1H), 5.13-5.03 (m, 1H), 4.77 (t, J=7.9 Hz, 1H), 4.50 (s, 1H), 4.44 (d, J=8.0 Hz, 1H), 4.29 (s, 2H), 4.20 (d, J=11.1 Hz, 1H), 3.91-3.77 (m, 6H), 3.67-3.56 (m, 3H), 3.55-3.47 (m, 4H), 3.33 (s, 2H), 2.91-2.77 (m, 2H), 2.60-2.48 (m, 5H), 2.13-2.02 (m, 1H), 1.48 (d, J=6.9 Hz, 3H), 1.08 (s, 9H). HRMS (ESI) m/z [M+H]+ calcd for C₄₂H₅₃F₂N₇O₇S₂ 870.34887, found 870.35050.

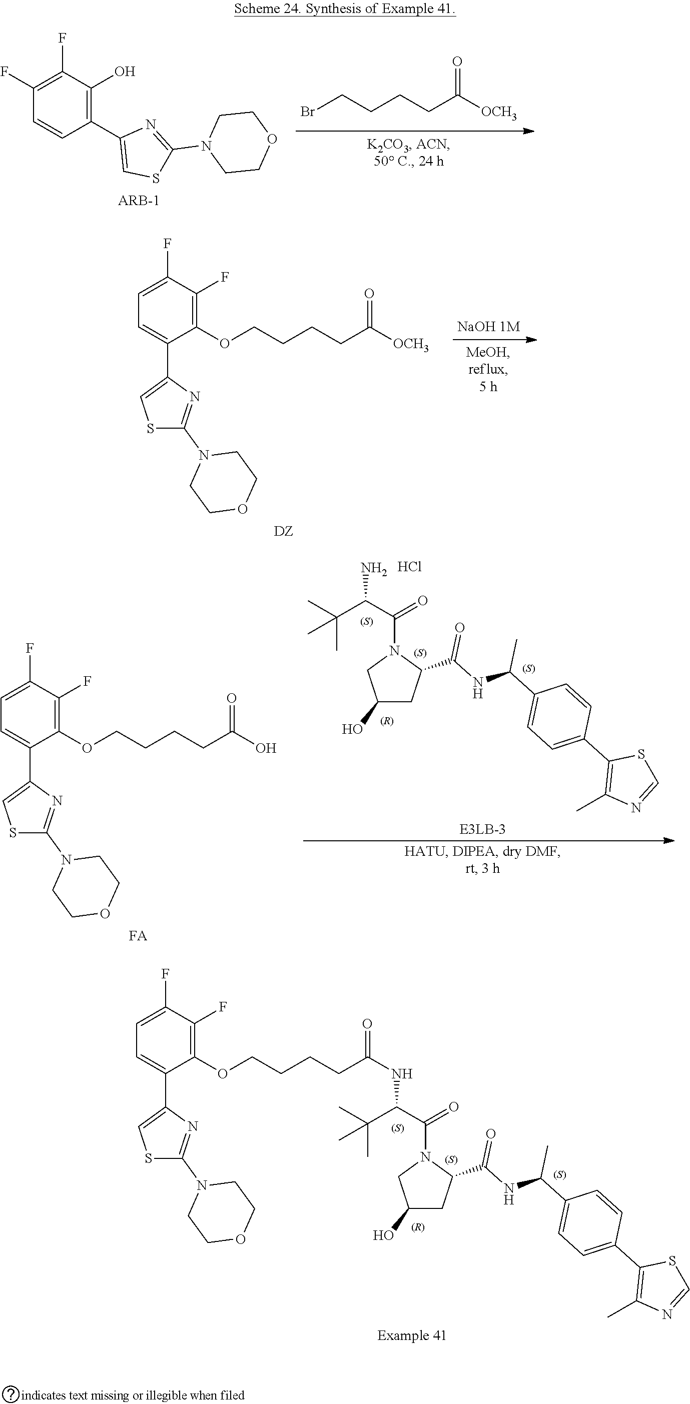

### Methyl 5-(2,3-difluoro-6-(2-morpholinothiazol-4-yl)phenoxy)pentanoate (DZ)

Methyl 5-bromopentanoate (0.030 mL, 0.428 mmol) was added to a stirred solution of 2,3-difluoro-6-(2-morpholinothiazol-4-yl)phenol (ARB-1) (0.064 g, 0.214 mmol) and K₂CO₃ (0.148 mL, 1.070 mmol) in ACN (3.0 mL). The solution was stirred at 50° C. for 24 h. After cooling, the reaction mixture was evaporated to dryness, diluted with water (20 mL) and extracted with EA (8 mL×3). The reunited organic layers were washed with brine (10 mL×1), dried over anhydrous Na₂SO₄ and then concentrated under reduced pressure to afford the titled compound (0.085 g, 97% yield) as a yellow oil. ¹H NMR (400 MHz, CDCl₃) δ 7.79 (ddd, J=8.8, 6.2, 2.3 Hz, 1H), 7.21 (s, 1H), 6.97-6.86 (m, 1H), 4.16-4.05 (m, 2H), 3.89-3.80 (m, 4H), 3.68 (s, 3H), 3.60-3.50 (m, 4H), 2.42-2.35 (m, 2H), 1.87-1.77 (m, 4H).

### 5-(2,3-Difluoro-6-(2-morpholinothiazol-4-yl)phenoxy)pentanoic acid (FA)

To the solution of methyl 5-(2,3-difluoro-6-(2-morpholinothiazol-4-yl)phenoxy)pentanoate (DZ) (0.088 g, 0.213 mmol) in MeOH (0.6 mL) at 0° C. was added a solution of sodium hydroxide 1M (0.610 mL). The resulting mixture was refluxed for 5 h. The organic solvent was removed under vacuo, the residue was diluted with ice-water (20 mL) and the pH was slowly adjusted to 2-3 with 2N HCl. The mixture was extracted with EA (8 mL×3). The reunited organic layers were washed with brine (10 mL×1), dried over anhydrous Na₂SO₄ and then concentrated under reduced pressure to afford the titled compound (0.056 g, 67% yield) as a white solid. ¹H NMR (400 MHz, DMSO-d₆) δ 12.03 (bs, 1H), 7.80 (ddd, J=8.8, 6.4, 2.1 Hz, 1H), 7.34 (s, 1H), 7.24-7.15 (m, 1H), 4.08 (t, J=6.2 Hz, 2H), 3.80-3.68 (m, 4H), 3.47-3.40 (m, 4H), 2.27 (t, J=7.3 Hz, 2H), 1.83-1.71 (m, 2H), 1.70-1.59 (m, 2H).

### (2S,4R)-1-((S)-2-(5-(2,3-difluoro-6-(2-morpholinothiazol-4-yl)phenoxy)pentanamido)-3,3-dimethylbutanoyl)-4-hydroxy-N—((S)-1-(4-(4-methylthiazol-5-yl)phenyl)ethyl)pyrrolidine-2-carboxamide (Example 41)

In an oven-dried round-bottom flask, under nitrogen atmosphere, to a stirred solution of 5-(2,3-difluoro-6-(2-morpholinothiazol-4-yl)phenoxy)pentanoic acid (FA) (0.047 g, 0.117 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (E3LB-3) (0.058 g, 0.117 mmol) and DIPEA (0.084 mL, 0.479 mmol) in dry DMF (1.0 mL) was added HATU (0.057 g, 0.150 mmol). Stirring was continued at rt 3 h. The reaction mixture was poured in ice-water yielding a precipitate which was collected by filtration, dried, and purified by flash column chromatography on SiO₂ (DCM/Acetone/MeOH, 77:20:3) affording the titled compound (23 mg, 23% yield) as white solid. ¹H NMR (400 MHz, DMSO-d₆) δ 8.98 (s, 1H), 8.37 (d, J=7.8 Hz, 1H), 7.89-7.77 (m, 2H), 7.45-7.35 (m, 4H), 7.34 (s, 1H), 7.23-7.15 (m, 1H), 5.09 (d, J=3.5 Hz, 1H), 4.96-4.88 (m, 1H), 4.54 (d, J=4.9 Hz, 1H), 4.42 (t, J=8.0 Hz, 1H), 4.27 (s, 1H), 4.09 (t, J=6.1 Hz, 2H), 3.77-3.70 (m, 4H), 3.66-3.56 (m, 2H), 3.48-3.40 (m, 4H), 2.45 (s, 3H), 2.38-2.27 (m, 1H), 2.22-2.10 (m, 1H), 2.04-1.96 (m, 1H), 1.82-1.60 (m, 5H), 1.37 (d, J=7.0 Hz, 3H), 0.93 (s, J=5.7 Hz, 9H). HRMS (ESI) m/z [M+H]+ calcd for C₄₁H₅₀F₂N₆O₆S₂ 825.32741, found 825.32833.

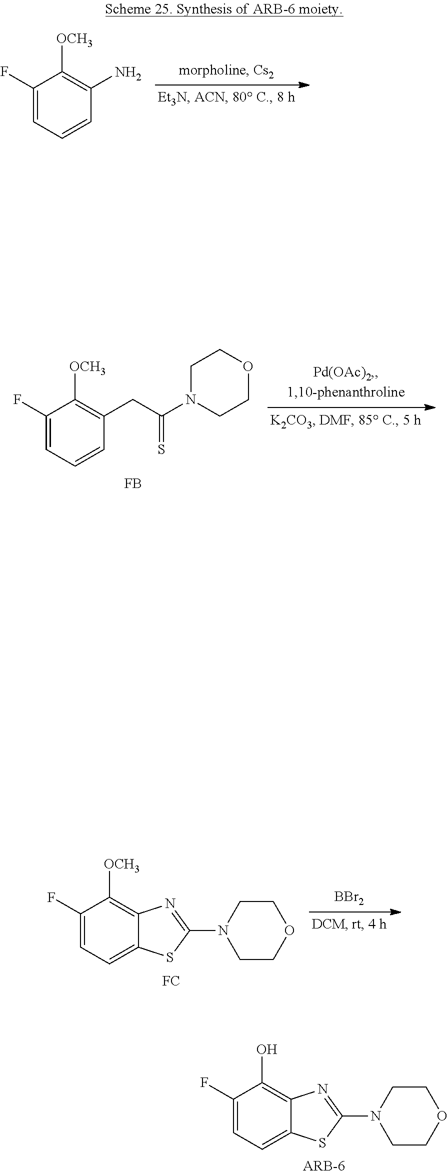

### N-(3-Fluoro-2-methoxyphenyl)morpholine-4-carbothioamide (FB)

To stirred of morpholine (0.250 mL, 2.835 mmol) and Et₃N (0.414 mL, 3.122 mmol) in ACN (4.0 mL) was added CS₂ (0.200 mL, 3.408 mmol) dropwise over a period of 5 min at 0° C., and the stirring was continued for 10 min at 0° C. After allowing the reaction mixture to warm to rt, stirring was continued for additional 10 min. Thus, 3-fluoro-2-methoxyaniline (0.171 mL, 1.417 mmol) was added and the reaction mixture was stirred at 80° C. for 8 h. The solvent was evaporated to dryness and the crude residue was diluted with 2N HCl (8.0 mL) yielding a brown precipitate, which was collected by filtration, washed with cyclohexane (16.0 mL) and then purified by automated flash chromatography on SiO₂ cartridge (PE/EA, 7:3) to give the title compound (0.185 g, 48%) as beige solid. ¹H NMR (400 MHz, DMSO-d₆) δ 9.05 (s, 1H), 7.17-7.08 (m, 1H), 7.08-6.98 (m, 2H), 3.94-3.86 (m, 4H), 3.81 (s, 3H), 3.69-3.62 (m, 4H); ¹³C NMR (101 MHz, DMSO-d₆) δ 183.17, 156.99, 154.56, 143.68 (d, J=11.5 Hz), 135.86 (d, J=4.1 Hz), 126.13 (d, J=2.8 Hz), 123.12 (d, J=9.1 Hz), 114.79 (d, J=19.0 Hz), 66.24 (2C), 61.43 (d, J=4.4 Hz), 48.94 (2C).

### 4-(5-Fluoro-4-methoxybenzo[d]thiazol-2-yl)morpholine (FC)

To the solution of N-(3-fluoro-2-methoxyphenyl)morpholine-4-carbothioamide (FB) (0.300 g, 1.109 mmol) in DMF (2.0 mL) was added K₂CO₃ (0.231 g, 1.664 mmol), 1,10-phenanthroline (0.030 g, 0.166 mmol) and Pd(OAc)₂ (0.020 g, 0.088 mmol) and the reaction mixture was stirred at 80° C. for 5 h. After cooling the mixture was filtered over Celite, and the filtrate was diluted with water (30 mL) and extracted with EA (10 mL×3). The reunited organic layers were washed with water (10 mL×2), brine (10 mL×1), dried over anhydrous Na₂SO₄ and then concentrated under reduced pressure to afford a crude residue which was purified by automated flash chromatography on SiO₂ cartridge (PE/EA, 70:30) to give the titled compound (0.125 g, 42% yield) as white solid. ¹H NMR (400 MHz, DMSO-d₆) δ 7.47 (dd, J=8.7, 4.7 Hz, 1H), 6.98 (dd, J=11.4, 8.7 Hz, 1H), 4.03 (s, 3H), 3.77-3.70 (m, 4H), 3.60-3.53 (m, 4H); ¹³C NMR (101 MHz, DMSO-d₆) δ 169.57, 154.44, 152.07, 146.37 (d, J=5.1 Hz), 138.21 (d, J=13.3 Hz), 127.60 (d, J=1.8 Hz), 115.93 (d, J=9.4 Hz), 110.39 (d, J=22.2 Hz), 65.92 (2C), 61.33 (d, J=1.9 Hz), 48.49 (2C).

### 5-Fluoro-2-morpholinobenzo[d]thiazol-4-ol (ARB-6)

Under nitrogen atmosphere, to the solution of 4-(5-fluoro-4-methoxybenzo[d]thiazol-2-yl)morpholine (FC) (0.050 g, 0.186 mmol) in dry DCM (2.0 mL) a 1.0 M BBr₃ solution in DCM (0.093 mL, 0.930 mmol) was added dropwise at 0° C. The reaction mixture was stirred at rt for 4 h and then quenched with MeOH (10 mL) and water (20 mL). The mixture was extracted with DCM (10 mL×3). The reunited organic layers were washed brine (10 mL×1), dried over anhydrous Na₂SO₄ and then concentrated under reduced pressure to afford a crude residue which was triturated with DEE and collected by filtration to give the titled compound (0.030 g, 63% yield) as white solid. ¹H NMR (400 MHz, DMSO-d₆) δ 9.54 (s, 1H), 7.19 (dd, J=8.6, 4.5 Hz, 1H), 6.92 (dd, J=11.3, 8.7 Hz, 1H), 3.77-3.70 (m, 4H), 3.59-3.51 (m, 4H); ¹³C NMR (101 MHz, DMSO-d₆) δ 168.79, 150.13 (d, J=233.5 Hz), 143.76 (d, J=6.5 Hz), 136.19 (d, J=14.8 Hz), 126.68 (d, J=1.6 Hz), 111.44 (d, J=8.9 Hz), 110.38 (d, J=21.5 Hz), 65.95 (2C), 48.57 (2C).

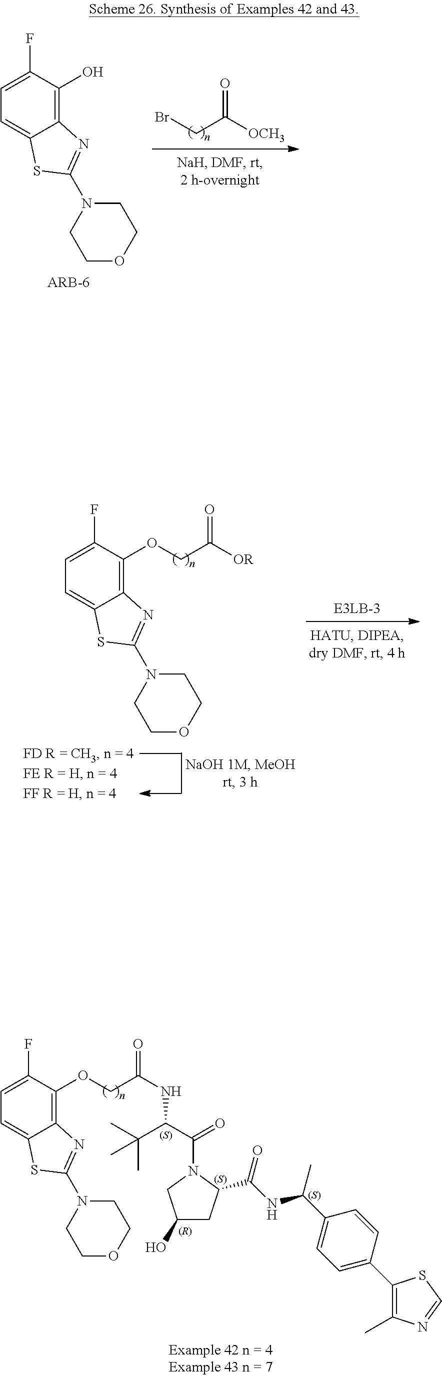

### Methyl 5-((5-fluoro-2-morpholinobenzo[d]thiazol-4-yl)oxy)pentanoate (FD)

Under nitrogen atmosphere, to the suspension of NaH 60% (0.028 g, 0.708 mmol) in dry DMF (0.5 mL) a solution of 5-fluoro-2-morpholinobenzo[d]thiazol-4-ol (ARB-6) (0.060 g, 0.236 mmol) in dry DMF (0.5 mL) was added dropwise at 0° C. After 30 min at 0° C., a solution of methyl 5-bromopentanoate (0.068 mL, 0.472 mmol) in dry DMF (0.5 mL) was added and the reaction mixture was stirred at 50° C. for 2 h. The mixture was quenched with EtOAc (5 mL) and water (20 mL) and extracted with EA (8 mL×3). The reunited organic layers were washed brine (10 mL×1), dried over anhydrous Na₂SO₄ and then concentrated under reduced pressure to afford a crude residue which was purified by automated flash chromatography on SiO₂ cartridge (PE/EA, 70:30) to afford the titled compound (0.050 g, 29% yield) as a colorless oil. ¹H NMR (400 MHz, CDCl₃) δ 7.19 (dd, J=8.6, 4.4 Hz, 1H), 6.86 (dd, J=11.0, 8.6 Hz, 1H), 4.35 (t, J=6.1 Hz, 2H), 3.89-3.79 (m, 4H), 3.66 (s, 3H), 3.65-3.58 (m, 4H), 2.44 (t, J=7.4 Hz, 2H), 1.96-1.77 (m, 4H).

### 8-((5-Fluoro-2-morpholinobenzo[d]thiazol-4-yl)oxy)octanoic acid (FE)

Under nitrogen atmosphere, to the suspension of NaH 60% (0.028 g, 0.708 mmol) in dry DMF (0.5 mL) a solution of 5-fluoro-2-morpholinobenzo[d]thiazol-4-ol (ARB-6) (0.060 g, 0.236 mmol) in dry DMF (0.5 mL) was added dropwise at 0° C. After 30 min at 0° C., a solution of ethyl 8-bromooctanoate (0.119 g, 0.472 mmol) in dry DMF (1.0 mL) was added and the reaction mixture was stirred at 50° C. overnight. The mixture was quenched with EtOAc (5 mL) and water (20 mL), the pH was slowly adjusted to 3-4 with 2N HCl, and extracted with EA (10 mL×3). The reunited organic layers were washed brine (10 mL×1), dried over anhydrous Na₂SO₄ and then concentrated under reduced pressure to afford the titled compound (0.042 g, 45% yield) as a white solid. ¹H NMR (400 MHz, CDCl₃) δ 7.18 (dd, J=8.6, 4.4 Hz, 1H), 6.86 (dd, J=11.0, 8.6 Hz, 1H), 4.34 (t, J=6.5 Hz, 2H), 3.85-3.80 (m, 4H), 3.65-3.59 (m, 4H), 2.37-2.31 (m, 2H), 1.83-1.73 (m, 2H), 1.67-1.62 (m, 2H), 1.55-1.48 (m, 2H), 1.36 (dd, J=7.4, 3.7 Hz, 4H).

### 5-((5-Fluoro-2-morpholinobenzo[d]thiazol-4-yl)oxy)pentanoic acid (DF)

To the solution of methyl 5-((5-fluoro-2-morpholinobenzo[d]thiazol-4-yl)oxy)pentanoate (DD) (0.034 g, 0.092 mmol) in MeOH (0.21 mL) at 0° C. was added a solution of sodium hydroxide 1M (0.23 mL). The resulting mixture was stirred at rt for 3 h. The organic solvent was removed under vacuo, the residue was diluted with ice-water (10 mL) and the pH was slowly adjusted to 2-3 with 2N HCl. The mixture was extracted with EA (5 mL×3). The reunited organic layers were washed with brine (10 mL×1), dried over anhydrous Na₂SO₄ and then concentrated under reduced pressure to afford the titled compound (0.024 g, 74% yield) as a white solid. ¹H NMR (400 MHz, CDCl₃) δ 7.19 (dd, J=8.6, 4.4 Hz, 1H), 6.87 (dd, J=11.1, 8.6 Hz, 1H), 4.37 (t, J=5.9 Hz, 2H), 3.87-3.79 (m, 4H), 3.66-3.60 (m, 4H), 2.52 (t, J=7.3 Hz, 2H), 2.00-1.82 (m, 4H).

### (2S,4R)-1-((S)-2-(5-((5-fluoro-2-morpholinobenzo[d]thiazol-4-yl)oxy)pentanamido)-3,3-dimethylbutanoyl)-4-hydroxy-N—((S)-1-(4-(4-methylthiazol-5-yl)phenyl)ethyl)pyrrolidine-2-carboxamide (Example 42)

In an oven-dried round-bottom flask, under nitrogen atmosphere, to a stirred solution of 5-((5-fluoro-2-morpholinobenzo[d]thiazol-4-yl)oxy)pentanoic acid (DF) (0.023 g, 0.065 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (E3LB-3) (0.031 g, 0.065 mmol) and DIPEA (0.046 mL, 0.260 mmol) in dry DMF (1.0 mL) was added HATU (0.031 g, 0.081 mmol). Stirring was continued at rt 4 h. The reaction mixture was diluted with water (20 mL) and extracted with EA (8 mL×3). The reunited organic layers were washed with water (8 mL×3), brine (10 mL×3), dried over anhydrous Na₂SO₄ and then concentrated under reduced pressure to give a crude residue, which was purified by preparative TLC on SiO₂ (DCM/MeOH, 95:5) affording the titled compound (13.0 mg, 25% yield) as white solid. ¹H NMR (400 MHz, CDCl₃) δ 8.69 (s, 1H), 7.52 (d, J=7.7 Hz, 1H), 7.45-7.33 (m, 4H), 7.19 (dd, J=8.6, 4.4 Hz, 1H), 6.86 (dd, J=11.2, 8.7 Hz, 1H), 6.50 (d, J=8.1 Hz, 1H), 5.12-5.02 (m, 1H), 4.75 (t, J=8.0 Hz, 1H), 4.53-4.47 (m, 2H), 4.46-4.38 (m, 1H), 4.34-4.26 (m, 1H), 4.20 (d, J=11.5 Hz, 1H), 3.87-3.78 (m, 4H), 3.68-3.52 (m 5H), 2.64-2.46 (m, 5H), 2.42-2.32 (m, 1H), 2.10-2.01 (m, 1H), 1.95-1.79 (m, 4H), 1.46 (d, J=6.9 Hz, 3H), 1.03 (s, 9H). HRMS (ESI) m/z [M+Na]+ calcd for C₃₉H₄₉FN₆O₆S₂ 803.30312, found 803.30448.

### (2S,4R)-1-((S)-2-(8-((5-fluoro-2-morpholinobenzo[d]thiazol-4-yl)oxy)octanamido)-3,3-dimethylbutanoyl)-4-hydroxy-N—((S)-1-(4-(4-methylthiazol-5-yl)phenyl)ethyl)pyrrolidine-2-carboxamide (Example 43)

In an oven-dried round-bottom flask, under nitrogen atmosphere, to a stirred solution of 8-((5-fluoro-2-morpholinobenzo[d]thiazol-4-yl)oxy)octanoic acid (DE) (0.032 g, 0.082 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (E3LB-3) (0.039 g, 0.082 mmol) and DIPEA (0.057 mL, 0.324 mmol) in dry DMF (1.0 mL) was added HATU (0.038 g, 0.101 mmol). Stirring was continued at rt 4 h. The reaction mixture was diluted with water (20 mL) and extracted with EA (8 mL×3). The reunited organic layers were washed with water (8 mL×3), brine (10 mL×3), dried over anhydrous Na₂SO₄ and then concentrated under reduced pressure to give a crude residue, which was purified by automated flash chromatography on SiO₂ cartridge (DCM/Acetone/MeOH, 85:10:5) to afford the titled compound (0.010 g, 15% yield) as a white solid. ¹H NMR (400 MHz, CDCl₃) δ 8.69 (s, 1H), 7.46-7.31 (m, 5H), 7.18 (dd, J=8.6, 4.4 Hz, 1H), 6.85 (dd, J=11.0, 8.6 Hz, 1H), 6.09 (d, J=8.5 Hz, 1H), 5.13-5.03 (m, 1H), 4.75 (t, J=7.9 Hz, 1H), 4.58-4.47 (m, 2H), 4.32 (t, J=6.6 Hz, 2H), 4.12 (d, J=11.1 Hz, 1H), 3.88-3.76 (m, 4H), 3.66-3.54 (m, 5H), 2.60-2.48 (m, 4H), 2.21 (t, J=7.3 Hz, 2H), 2.09-2.00 (m, 1H), 1.81-1.71 (m, 2H), 1.66-1.57 (m, 2H), 1.54-1.44 (m, 5H), 1.39-1.27 (m, 4H), 1.05 (s, 9H). HRMS (ESI) m/z [M+H]+ calcd for C₄₂H₅₅FN₆O₆S₂ 823.36813, found 823.37064.

### Experimental Example 1: 22Rv1 Cell Proliferation Assays

The human prostate cancer cell line, 22Rv1 has been reported to express a high level of AR-V7. Thus, 22Rv1 was seeded at 50,000 cells/well on a 24-well plate in quadruplicates and treated with test compound in concentrations ranging up to 20 μM for four days. Standard culture media was RPMI-1640 supplemented with 10% fetal bovine serum. The test compound initially was dissolved in DMSO at 50 mM. This stock solution was then diluted as needed for the indicated concentrations. At the end of the four-day period, cells were harvested using 1% trypsin and counted using an automated cell counter.

The results as shown in Table 1 below demonstrate that the test compounds decreased cell count in a concentration dependent manner. In the Table: (+)—the cell count decreased between 0 and 20%; (++)—the cell count decreased less than 50%; (+++)—the cell count decreased by more than 50%.

TABLE 1

| Compound | 22Rv1 cell count |
|---|---|
| of | decrease at 10 μM |
| Example | for 48 hours |
| 1 | ++ |
| 2 | + |
| 3 | ++ |
| 4 | + |
| 5 | + |
| 6 | + |
| 7 | + |
| 8 | + |
| 9 | + |
| 10 | + |
| 11 | + |
| 12 | + |
| 13 | + |
| 14 | + |
| 15 | ++ |
| 16 | ++ |
| 17 | ++ |
| 18 | ++ |
| 19 | ++ |
| 20 | ++ |
| 21 | ++ |
| 22 | + |
| 23 | + |
| 24 | +++ |
| 25 | +++ |
| 26 | +++ |
| 27 | +++ |
| 28 | +++ |
| 29 | ++ |
| 30 | ++ |
| 31 | +++ |
| 32 | + |
| 33 | + |
| 34 | ++ |
| 35 | +++ |
| 36 | +++ |

### Experimental Example 2: Immunoblot

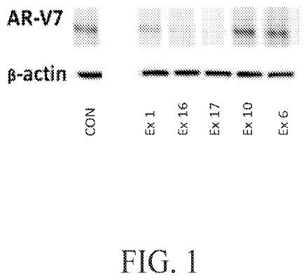

Immunoblot was carried out to determine the effect of the test compound on AR-V7. 22Rv1 was plated at 200,000 cell/well on a 6-well plate and cultured as described with 10 μM test compound. After four days of treatment, cells were harvested using a cell scraper and lysed in a standard fashion using SDS. After removing debris via centrifuge, 30 μg of protein were loaded onto SDS-PAGE gel. After electrophoresis, protein was transferred to a nylon membrane and ECL was carried out using primary antibody against AR-V7 (Thermofisher Scientific, cat #NC0752138). Protein bands were visualized using the commercially available Enhanced Chemiluminescence (ECL) kit (Thermofisher). As shown in FIG. 1, the results demonstrated a dramatically decreased level of AR-V7 protein.

### Experimental Example 3

Compounds according to the present disclosure (“Test Compound”) are tested for in vitro efficacy against various CaP cell lines.

Cell Culture. Human CaP cell lines, LNCaP, 22Rv1, VCaP, PC3, and DU145 are obtained from the American Type Culture Collection (ATCC) and maintained in the standard culture media: RPMI-1640 supplemented with 10% fetal bovine serum (FBS). LNCaP, 22Rv1, and VCaP are androgen-responsive cell lines, while PC3 and DU145 are not. To establish SAT resistant CaP cell lines, LNCaP, 22Rv1, and VCaP are treated continuously with 10-50 μM abiraterone, apalutamide, darolutamide, or enzalutamide. After 3-6 months, stable cell lines are established and designated as LNCaP-Abi^{R}, LNCaP-Apa^{R}, LNCaP-Daro^{R}, LNCaP-Enz^{R}, VCaP-Abi^{R}, VCaP-Apa^{R}, VCaP-Daro^{R}, VCaP-Enz^{R}, 22Rv1-Abi^{R}, 22Rv1-Apa^{R}, 22Rv1-Daro^{R} and 22Rv1-Enz^{R}. Unless otherwise specified, the standard culture media for these SAT-resistant cell lines included 10 μM of their respective SAT. For the proteasome inhibitor study, the inhibitors MG132 and Epoxomicin are used. The E3 ligase inhibitors Heclin, Nutlin 3a, Thalidomide, and VH298 are used. Cell lines obtained from ATCC are confirmed by checking their morphology using optical microscopy, establishing baselines for cell proliferation, verifying species of origin using isoenzymology, and characterizing the cell's DNA fingerprint using short tandem repeat (STR) profiling. Mycoplasma contamination is also assessed using a PCR based detection system.

Apoptosis Assay. An apoptosis assay is carried out using the Thermo Fisher ApoDETECT Annexin V-FITC kit following the protocol recommended by the vendor. Briefly, after treatment with 1 μM of Test Compound for 3 to 24 hours, cells are fixed with 80% ethanol and washed with PBS three times. Then, fixed cells are incubated with Annexin V-FITC in PBS solution for 30 minutes at room temperature. After washing three times with PBS, cells are treated with 300 nM DAPI in PBS for 5 minutes at room temperature. Finally, after washing three times with PBS, mounting solution is added and the cells are visualized using immunofluorescence microscopy. Next, a TUNEL assay is performed using Promega DeadEnd Fluorometric TUNEL system. After treatment with Test Compound and fixation as described above in the Annexin-V experiment, 100 μl of equilibration buffer is incubated for 10 min. Then, 50 μl of TdT reaction mix is added and incubated for 60 min at 37° C. in a humidified chamber. Finally, stop solution is added and samples are mounted on slides using mounting medium. To assess non-specific cytotoxicity, an LDH assay kit is used.

Transient Transfections. One μg of a plasmid containing cDNA of AR-V7 or AR-FL is transfected into indicated the CaP cell lines on 6-well plates. Three μl of lipofectamine 3000 is used for each transfection.

Immunoblot Analysis. CaP cells are collected and lysed with the lysis buffer (20 mM Tris-HCl (pH 7.5), 150 mM NaCl, 1 mM Na₂EDTA, 1 mM EGTA, 1% Triton, 2.5 mM sodium pyrophosphate, 1 mM beta-glycerophosphate, 1 mM Na₃VO₄, and 1 μg/ml leupeptin) containing 1 mM phenylmethylsulfonyl fluoride (PMSF). Cell lysates are then centrifuged and protein in the supernatant is quantified. After separating 25-50 μg of protein using SDS-PAGE, samples are incubated with AR-V7, GR, PR, ERα, AR-FL, ubiquitin, or β-actin antibodies. For AR-V7, AR-FL, PR, GR, and ERα immunoblots, primary antibody is diluted 1:1000 in 5% skim milk. For the β-actin immunoblot, 1:10000 diluted primary antibody is used. All membranes are incubated overnight at 4° C. Following the incubation with appropriate secondary antibody, immunoblots are analyzed using SuperSignal West Femto Maximum Sensitivity Substrate (ThermoFisher).

In Vivo Study. To explore the therapeutic potential of Test Compound, 22Rv1, 22Rv1-Enz^{R}, VCaP, and VCaP-Enz^{R} are injected into nu/nu immunodeficient mice. When the resulting tumors reached an average size of 3 mm in diameter, all animals are surgically castrated via bilateral orchiectomy and divided into four groups of five mice each. For anesthesia, 3% isoflurane gas inhalation is used. Tumor size was measured using calipers and tumor volume was calculated using the formula: tumor volume=length×width²/0.361.

Mice are then treated daily with Test Compound with or without enzalutamide via the indicated route (intratumoral, intraperitoneal, or oral) for five to six weeks. At the end of the study, all animals are sacrificed and tumors are harvested and analyzed. Statistical significance is calculated using the Student's t-test for paired comparisons of experimental groups and, where appropriate, by Wilcoxon rank sum test, and by 2-way ANOVA. In vitro experiments are repeated a minimum of three times.

Results. Treatment of the AR-V7-expressing CaP cell line 22Rv1 with the Compound of Example 16 (0, 0.01, 0.1, 1 and 10 μM) for 24 hours, immunoblot demonstrated decreased AR-V7 and AR-FL protein expression levels in 22Rv1 cells starting at concentration as low as 0.1 μM and 1 μM, respectively. Example 16's effect on AR-V7 and AR-FL protein levels was concentration-dependent and AR-specific, as there was no visible effect on the expression levels of the proteins glucocorticoid receptor (GR), progesterone receptor (PR) a and b, and estrogen receptor alpha (ER). The concentration of Compound of Example 16 at which 50% of AR-V7 and AR-FL degraded in 24 hours (DC₅₀) is determined to be 0.37 and 2 μM, respectively. These results demonstrate that the Compound is able to degrade both AR-V7 and AR-FL, although the degradation effect is more efficient against AR-V7 compared to AR-FL.

Along with degrading AR-V7 and AR-FL, in the cell proliferation assay, Compound of Example 16 decreased the cell count of 22Rv1 in a concentration dependent manner over 6 days (cell counts are approximately 90%, 70% and 65% of control at 0.01, 0.1 and 1 μM, respectively). As additional controls, constructs comprising Example 16's DBD binding motif with its linker (Control 1, C1) and Example 16's VHL domain with its linker (Control 2, C2) are prepared, as shown below:

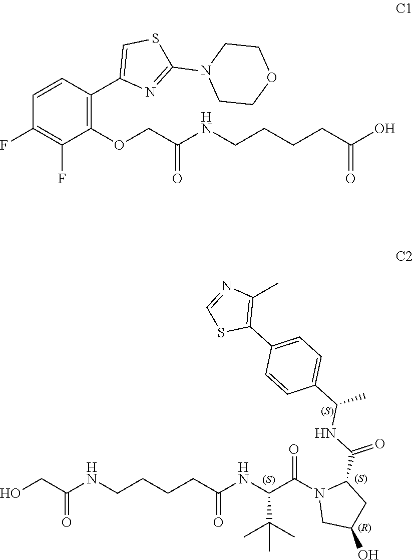

Treatment with C1 or C2 does not result in any significant changes in cell count at up to 1 μM concentration compared to the control (cell counts are 95-105% of control cell counts).

In another set of experiments, several cell lines (22Rv1, PC3, DU145, LNCaP, VCaP) are compared side to side for the effect of the Compound of Example 16. At 1 μM of Compound of Example 16, 22Rv1 cell count is approximately 40% of the control. The Compound only inhibits the proliferation of androgen-responsive cells (22Rv1, LNCaP, and VCaP). The AR-negative cell lines, PC3 and DU145, are not affected by the Compound. Upon transfecting AR-V7 or AR-FL into 22Rv1 cells, there is seen a partial resistance to the Compound's inhibitory effect (at 1 μM). However, with the transfection of both AR-V7 and AR-FL, the Compound's effect was completely abrogated in 22Rv1 cells. These results suggest that the Compound of Example 16 inhibits CaP cellular proliferation by degrading AR-V7 and AR-FL.

After treating 22Rv1 cells with Compound 16 at 1 μM, the annexin-V assay is carried out to assess the effect on apoptosis over a 0 to 48 hours period. Starting approximately three hours after treatment, an increase in annexin-V staining is observed via fluorescence microscopy, and it continues to increase through 48 hours. This result is confirmed by the TUNEL assay. As a negative control, the C1 control is compared, and no change in apoptosis is observed. In addition, using the lactate dehydrogenase (LDH) assay it is observed that the Compound at up to 20 μM had no non-specific cytotoxic effect in 22Rv1 after six days treatment (0.01, 0.1, 1 10 and 20 μM tested).

To determine the mechanism of the cell proliferation inhibition, 22Rv1 cells are pretreated for 2 hours with the proteasome inhibitors MG-132 (5 μM) and epoxomicin (1 μM) prior to treatment with Compound of Example 16 at 1 μM. It is found that AR-V7 degradation is completely blocked, as shown by immunoblotting. The E3 ubiquitin ligase inhibitors are also examined: VH 298 (VHL inhibitor; 20 μM), heclin (HECT inhibitor; 10 μM), nutlin 3a (MDM2 inhibitor; 0.1 μM), and thalidomide (cereblon inhibitor; 10 μM). It is found that when 22Rv1 cells are pretreated with each of these inhibitors for two hours prior to incubation with 1 μM Compound of Example 16, only VH298 pretreatment inhibits AR-V7 degradation.

To determine the ubiquitination status of AR proteins, 22Rv1 is pretreated with MG132 for two hours prior to adding Compound of Example 16 (at 1 μM). Immunoblot analysis demonstrates polyubiquitination of AR-V7 starting at 6 hours after Compound treatment. AR-FL polyubiquitination is also detected, but occurs significantly later, at 24 hours after treatment. Collectively, these results suggest that the Compound of Example 16 stimulates AR-V7 and AR-FL ubiquitination and degradation specifically via the VHL E3 ligase/proteasome axis.

To assess the potential therapeutic role of the Compound, twelve human CaP cell lines are generated that are resistant to the four FDA-approved SAT agents: abiraterone, apalutamide, enzalutamide, and darolutamide. Specifically, LNCaP, VCaP, and 22Rv1 cells are cultured with each of the SAT agents for three to six months until resistance emerges. The resulting cells are designated LNCaP-Abi^{R}, LNCaP-Apal^{R}, LNCaP-Darol^{R}, LNCaP-Enz^{R}, VcaP-Abi^{R}, VcaP-Apal^{R}, VcaP-Darol^{R}, VcaP-Enz^{R}, 22Rv1-Abi^{R}, 22Rv1-Apal^{R}, 22Rv1-Darol^{R}, and 22Rv1-Enz^{R}. Quantitative PCR demonstrates that all twelve SAT-resistant CaP cell lines express decreased and increased mRNA and protein levels of AR-FL and AR-V7, respectively. Using the cell proliferation assay above, it is found that all twelve cell lines' cell counts decreased after treatment with the Compound of Example 16 at 1 μM for 6 days (cell counts were reduced generally to 6-80% of control for all lines, except 22Rv1-EnzR, which was decreased to about 45% of control). Immunoblot assays with 22Rv1, VCaP and LNCaP cells demonstrate that the Compound decreases the protein expression levels of both AR-V7 and AR-FL in the parental and SAT-resistant cells. In all three cell lines, the effect on the protein levels of AR-V7 is greater than that on AR-FL. As negative controls, the AR-negative PC3 and DU145 cells are also compared. As predicted based on the suspected mechanism of action, the Compound had no major effect on the mRNA levels of AR-FL and AR-V7 in these cells.

The in vivo effects of the Compound of Example 16 are assessed in mice using enzalutamide-resistant CaP xenografts. First, 22Rv1-Enz^{R} tumor xenografts are established in nu/nu mice. Upon CaP xenograft formation (average diameter of 3 mm), mice are randomized into controls or treatment. Control mice are treated with 100 μl vehicle containing 10 mg/ml enzalutamide (n=5). The treatment group is injected with 100 μl vehicle containing 2.5 mg/ml Compound of Example 16 and 10 mg/ml enzalutamide daily directly into tumors (n=5) and tumor volumes were followed. The results demonstrate that mice treated with the Compound have a significantly smaller tumor volume at the end of 5 weeks (approximately 500 mm³ for treatment, and 800 mm³ for control). During the treatment period, weight of the mice did not significantly change, suggesting that the Compound does not have major toxicity in mice. Tumors are harvested and analyzed at the end of the study period. It is found that immunoblot demonstrates a significant decrease in AR-V7 and AR-FL protein levels in all treated tumors. Similar results are obtained when the Compound of Example 16 is injected intratumorally into VCaP-Enz^{R} xenografts.

To test whether the Compound is active systemically, an identical study is carried out as above except for delivering the Compound intraperitoneally (IP) or orally for four weeks. Enzalutamide-resistant CaP tumor xenografts are established in twelve mice and three each are assigned to the following four groups: control IP (vehicle), control oral (vehicle), 8.3 mg/kg IP, and 8.3 mg/kg oral. Because these cells are resistant to enzalutamide, all animals are administered enzalutamide. The results demonstrate that the Compound is effective when administered IP (tumor size approximately 1000 mm³ for treatment group, and 2200 mm³ for control group). Again, no significant change in weight was detected following treatment. Immunoblot shows that IP administration of the Compound decreased AR-V7 and AR-FL protein expression in Enz^{R} CaP tumors. Similar results are found when the Compound is delivered orally (PO) (tumor size approximately 700 mm³ for treatment group, and 2200 mm³ for control group).

Some of the above studies are carried out on additional compounds of the disclosure. For example, it is found that for the Compound of Example 26, the concentration of Compound at which 50% of AR-V7 and AR-FL is degraded in 24 hours (DC₅₀), as determined by immunoblot, is less than 50 nM and less than 500 nM, respectively. In cell proliferation assays, the Compounds of Examples 26, 34 and 35 at 10 μM reduces cell counts by substantially more than 50% after 4 days of treatment of 22Rv1-Enz^{R} cells, an effect substantially blocked by pretreatment with MG-132 (5 μM). Similar results are obtained for the Compound of Example 24. In the in vivo mouse tumor model described above, using 22Rv1-Enz^{R} tumors, the Compound of Example 26 substantially reduces tumor mass beginning at around 3 weeks of treatment through 7 weeks of treatment at 8.3 mg/kg and 0.83 mg/kg (tumor mass at 7 weeks, control approx. 1500 mm³; treatment groups <600 mm³). No significant changes in animal body mass observed.

### Experimental Example 4: 22Rv1-Enz^{R} Cell Proliferation Assays

Selected compounds were tested in the human prostate cancer cell line, 22Rv1-Enz^{R} as described in Geun Taek Lee et al., Molecular Cancer Therapeutics, 20:490-9 (2021). These procedures are similar to that described in Experimental Example 1, above, except that the assay conditions are a 6-day culture with cell media changed every 24 hours.

The results, as shown in Table 2 below (half maximal growth inhibition constant, GIC₅₀) demonstrate that the test compounds inhibited proliferation in a concentration dependent manner. In the Table: (+)—GIC₅₀<20 μM; (++)—GIC₅₀<10 μM; (+++)—GIC₅₀<1 μM.

TABLE 2

| Compound | 22Rv1-Enz^{R} |
|---|---|
| of | proliferation assay |
| Example | GIC₅₀ μM |
| 1 | + |
| 3 | + |
| 16 | +++ |
| 37 | +++ |
| 38 | + |
| 39 | + |
| 40 | +++ |
| 41 | ++ |
| 42 | ++ |
| 43 | + |

1. A compound having a chemical structure ARB-L-E3LB, wherein ARB is an AR binding moiety that does not bind to a ligand binding domain, E3LB is an E3 ligase binding moiety, and L is a linker coupling the AR binding moiety to the E3 ligase binding moiety, and wherein:
the AR binding moiety is selected from: 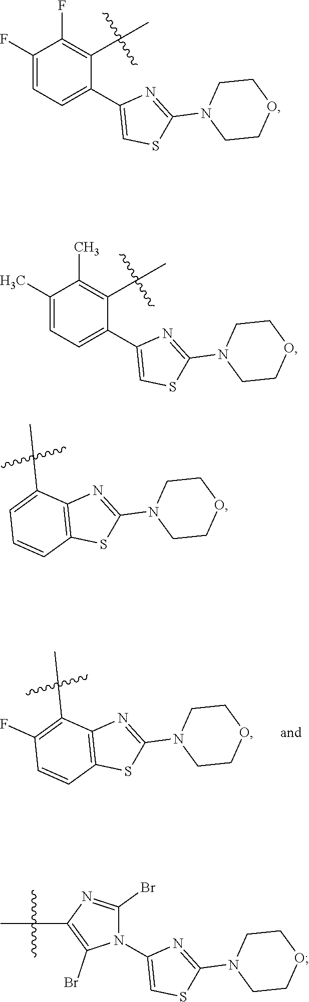 [SMILES: *c1c(-c2csc(N3CCOCC3)n2)ccc(F)c1F ; *c1c(-c2csc(N3CCOCC3)n2)ccc(C)c1C ; *c1cccc2sc(N3CCOCC3)nc12 ; *c1c(F)ccc2sc(N3CCOCC3)nc12 ; "and" ; *c1nc(Br)n(-c2csc(N3CCOCC3)n2)c1Br]
and
wherein the E3 ligase binding moiety is selected from:
(a) a structure selected from the group consisting of: 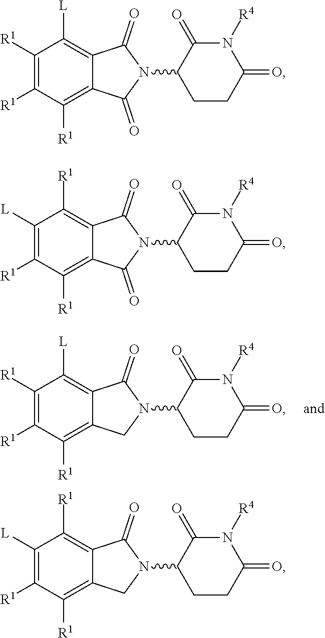
wherein “ [symbol] ” in the above structures, represents a bond that may be stereospecific ((R) or (S)), or non-stereospecific, optionally wherein the bond is oriented to form the (R)-chiral carbon, and wherein:
R¹ is each independently H or halo (e.g., F); and
R⁴ is H; or
(b) 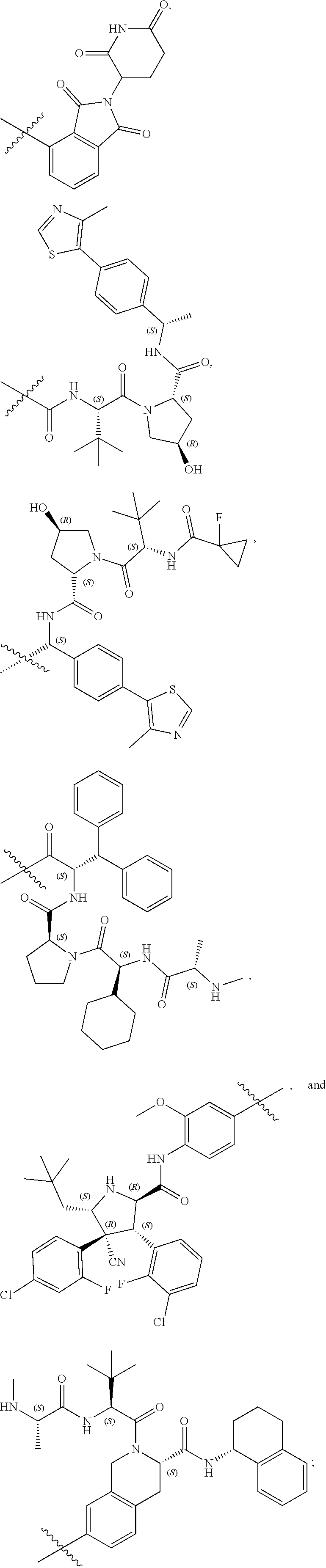
and
wherein the linker (“L” or “Link”) comprises a chemical structure represented by -A_{q}-, in which q is an integer greater than 1, and A is independently selected from the group consisting of a bond, CR^{L1}R^{L2}, O, NR^{L3}, CONR^{L3}, and CO; wherein R^{L1}, R^{L2}, and R^{L3} are each independently selected from the group consisting of H, halo, and C₁₋₈ alkyl.

2. The compound of claim 1, wherein the linker group (e.g., “L” or “Link”) is selected from: 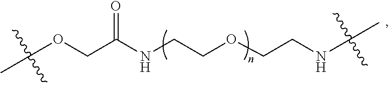
wherein n is from 1-5; 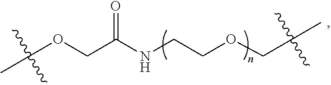
wherein n is from 1-5; 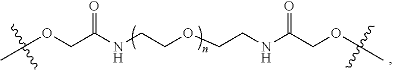
wherein n is from 1-5; 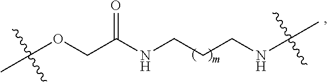
wherein m is from 0-12; 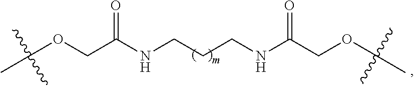
wherein m is rom 0-12; 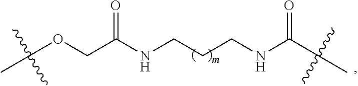
wherein m is from 0-12; 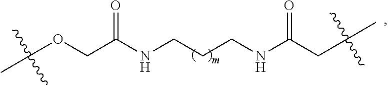
wherein m is from 2-4; 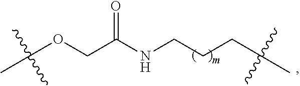
wherein m is from 0-12; 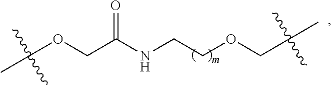
wherein n is from 0-10; 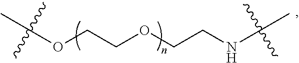
wherein n is from 1-5; 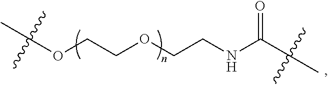
wherein n is from 1-5; 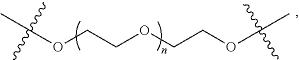
wherein n is from 1-5 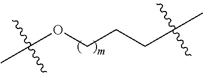
wherein m is from 0-10; 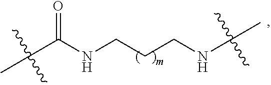
wherein m is from 0-10; 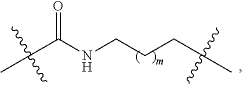
wherein m is from 0-10; 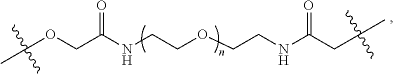
wherein n is from 1-5; 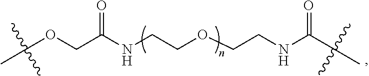
wherein n is from 1-5; 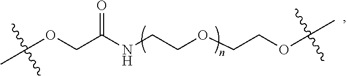
wherein n is from 1-5; 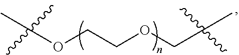
wherein n is from 1-5; 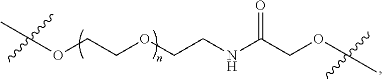
wherein n is from 1-5; 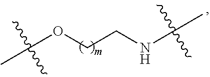 [SMILES: *NCCO*]
n wherein m is from 1-12; 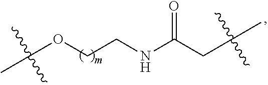
wherein m is from 1-12; 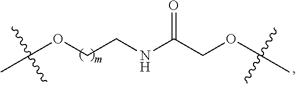
wherein m is from 1-12; and 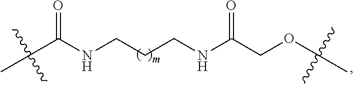
wherein m is from 0-10.

3. The compound of claim 1, wherein the linker group (e.g., “L” or “Link”) is selected from: 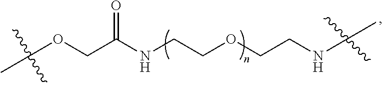
wherein n is from 2-4; 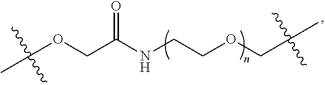
wherein n is from 2-4; 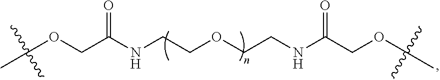
wherein n is from 2-3; 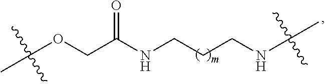
wherein m is from 2-8; 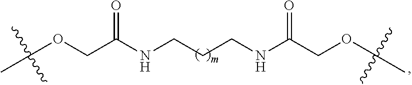
wherein m is from 4-8; 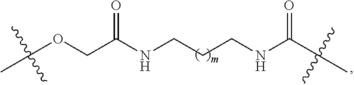
wherein m is from 2-4; 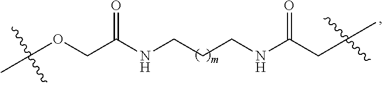
wherein m is from 2-4; 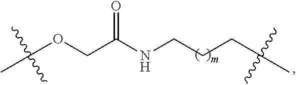
wherein m is from 1-4; 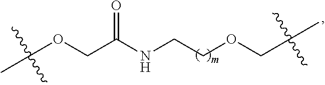
wherein m is from 1-4; 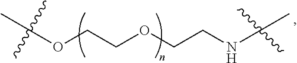
wherein n is from 2-4; 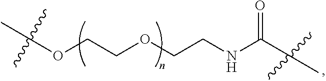
wherein n is from 1-3; 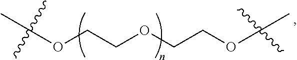
wherein n is from 1-2; 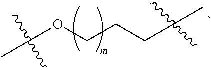
wherein m is from 4-6; 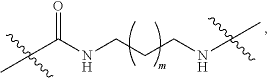
wherein m is from 2-4; and 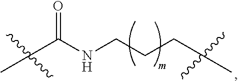
wherein m is from 2-4.

4. The compound of claim 1, wherein the linker group (e.g., “L” or “Link”) is selected from: 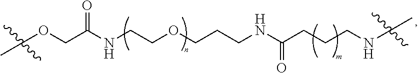
wherein n is from 0-4 (e.g., 1) and m is from 0-4 (e.g., 0); 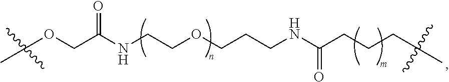
wherein n is from 0-4 (e.g., 1) and m is 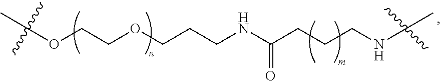
wherein n is from 0-4 (e.g., 1) and m is 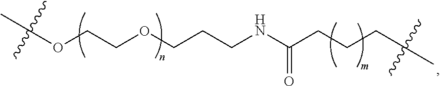
wherein n is from 0-4 (e.g., 1) and m is from 0-4 (e.g., 0); 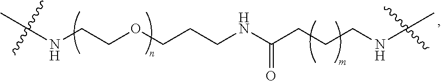
wherein n is from 0-4 (e.g., 1) and m is from 0-4 (e.g., 0); 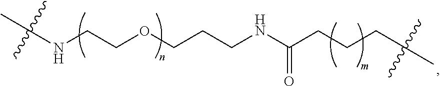
wherein n is from 0-4 (e.g., 1) and m is from 0-4 (e.g., 0); 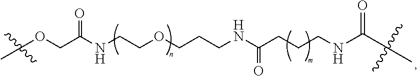
wherein n is from 0-4 (e.g., 1) and m is from 0-4 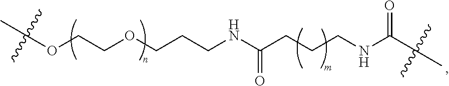
(e.g., 0); 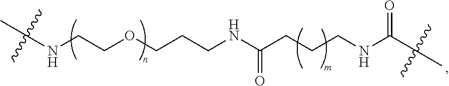
wherein n is from 0-4 (e.g., 1) and m is from 0-4 (e.g., 0); 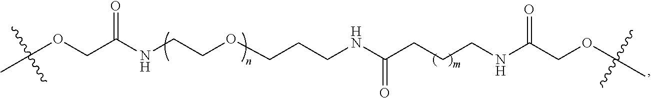
wherein n is from 0-4 (e.g., 1) and m is from 0-4 (e.g., 0); 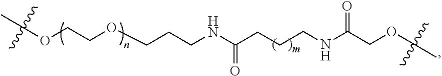
wherein n is from 0-4 (e.g., 1) and m is from 0-4 (e.g., 0); 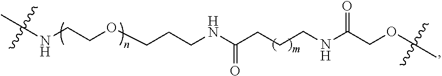
wherein n is from 0-4 (e.g., 1) and m is from 0-4 (e.g., 0); 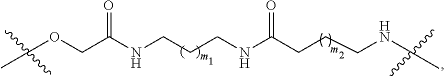
wherein m₁ is from 0-6 (e.g., 0 or 1) and m₂ is from 0-4 (e.g., 0); 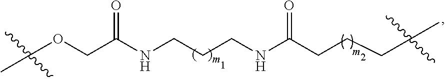
wherein m₁ is from 0-6 (e.g., 0 or 1) and m₂ is from 0-4 (e.g., 0); 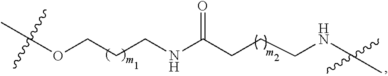
wherein m₁ is from 0-6 (e.g., 0 or 1) and m₂ is from 0-4 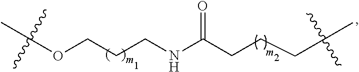
wherein m₁ is from 0-6 (e.g., 0 or 1) and m₂ is from 0-4 (e.g., 0); 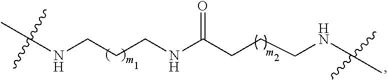
wherein m₁ is from 0-6 (e.g., 0 or 1) and m₂ is from 0-4 (e.g., 0); 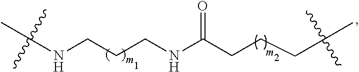
wherein m₁ is from 0-6 (e.g., 0 or 1) and m₂ is from 0-4 (e.g., 0); 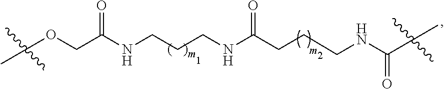
wherein m₁ is from 0-6 (e.g., 0 or 1) and m₂ is from 0-4 (e.g., 0); 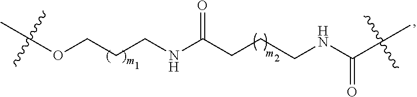
wherein m₁ is from 0-6 (e.g., 0 or 1) and m₂ is from 0-4 (e.g. 0 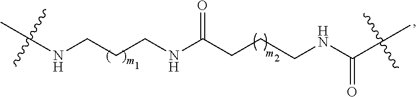
wherein m₁ is from 0-6 (e.g., 0 or 1) and m₂ is from 0-4 (e.g., 0); 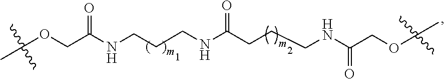
wherein m₁ is from 0-6 (e.g., 0 or 1) and m₂ is from 0-4 (e.g., 0); 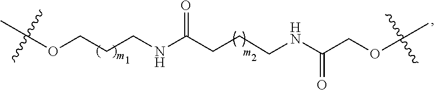
wherein m₁ is from 0-6 (e.g., 0 or 1) and m₂ is from 0-4 (e.g., 0); 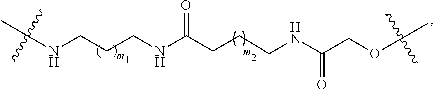
wherein m₁ is from 0-6 (e.g., 0 or 1) and m₂ is from 0-4 (e.g., 0).

5. The compound of claim 1, wherein the linker group (e.g., “L” or “Link”) is selected from: 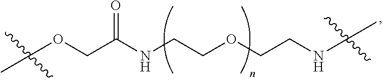
wherein n is from 1 to 4 (e.g., 2, 3, or 4), 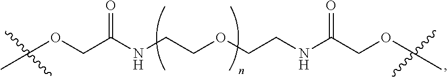
wherein n is from 1 to 4 (e.g., 2, 3, or 4); 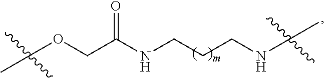
wherein m is from 2 to 8 (e.g., 4, 6, or 8); 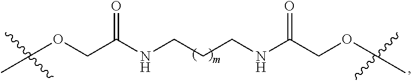
wherein m is from 2 to 8 (e.g., 4, 6, or 8); 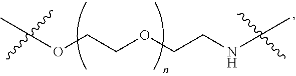
wherein n is from 1 to 4 (e.g., 2, 3, or 4), 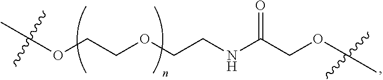
wherein n is from 1 to 4 (e.g., 2, 3, or 4); 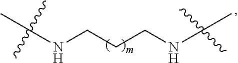
wherein m is from 2 to 8 (e.g., 4, 6, or 8); 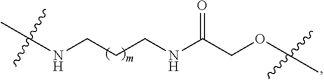
wherein m is from 2 to 8 (e.g., 4, 6, or 8); 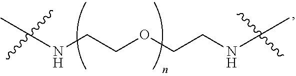
wherein n is from 1 to 4 (e.g., 2, 3, or 4); and 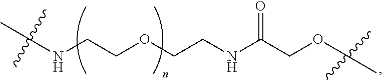
wherein n is from 1 to 4 (e.g., 2, 3, or 4).

6. The compound of claim 5, wherein the compound comprises:
the AR binding moiety 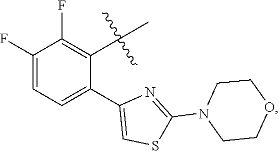 [SMILES: *c1c(-c2csc(N3CCOCC3)n2)ccc(F)c1F]
and
wherein the compound has an E3 ligase binding moiety selected from: 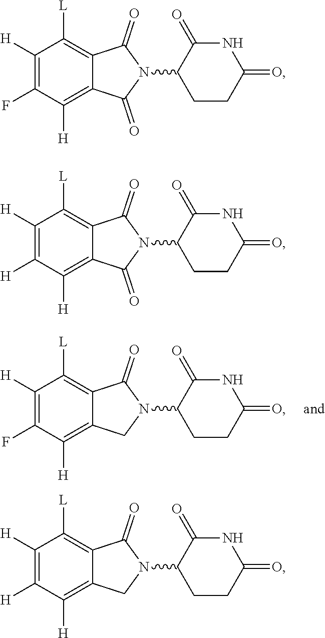
optionally wherein, in each of said compounds, the “ [symbol] ” bond is oriented to form the (R)-chiral carbon.

7. The compound of claim 1, wherein the linker group (e.g., “L” or “Link”) is selected from: 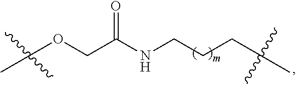
wherein m is from 0-6 (e.g., 1, 2, 3, or 4); 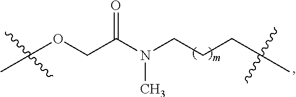
wherein m is from 0-6 (e.g., 1, 2, 3, or 4); 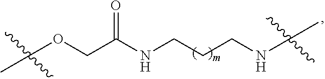
wherein m is from 0-6 (e.g., 1, 2, 3, or 4); 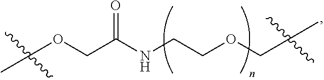
wherein n is from 1-5 (e.g., 1 or 2); 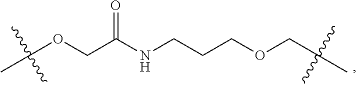
wherein n is from 1-5; 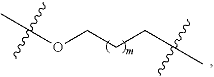
wherein m is from 0-6 (e.g., 2, 3, 4, or 5); 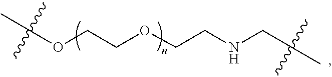
wherein n is from 1-6 (e.g., 1, 2, or 3); 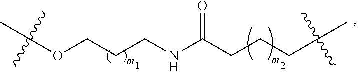
wherein m₁ is from 0-6 (e.g., 1) and m₂ is from 0-4 (e.g., 0); and 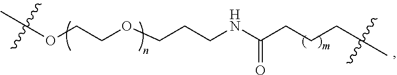
wherein n is from 0-4 (e.g., 1) and m is from 0-4 (e.g., 0).

8. The compound of claim 7, wherein the compound comprises:
the AR binding moiety 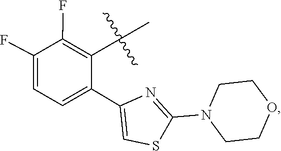 [SMILES: *c1c(-c2csc(N3CCOCC3)n2)ccc(F)c1F]
and
wherein the compound has the E3 ligase binding moiety

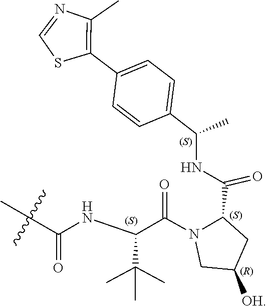

9. The compound of claim 7, wherein the compound comprises:
the AR binding moiety 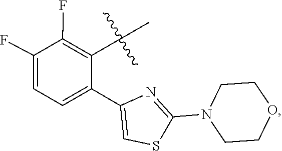 [SMILES: *c1c(-c2csc(N3CCOCC3)n2)ccc(F)c1F]
and
wherein the compound has the E3 ligase binding

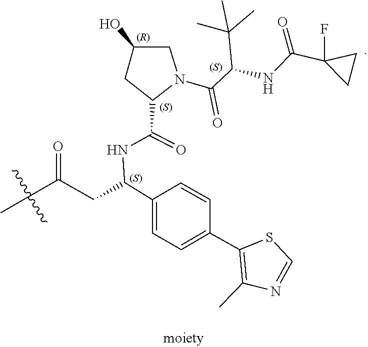

10. The compound of claim 1, wherein the linker group (e.g., “L” or “Link”) is selected from: 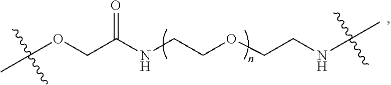
wherein n is from 1 to 4 (e.g., 1, 2, or 3); 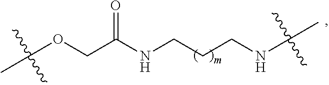
wherein m is from 2 to 8 (e.g., 2, 4, or 6); 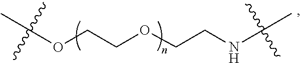
wherein n is 1 to 4 (e.g., 2 or 3); and 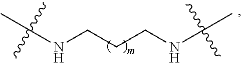
wherein m is from 2 to 8 (e.g., 2, 4, or 6).

11. The compound of claim 10, wherein the compound comprises:
the AR binding moiety 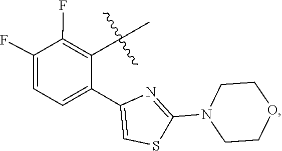 [SMILES: *c1c(-c2csc(N3CCOCC3)n2)ccc(F)c1F]
and
wherein the compound has the E3 ligase binding moiety

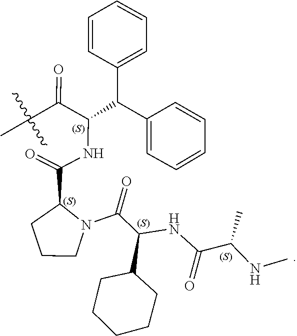

12. A compound selected from the group consisting of:

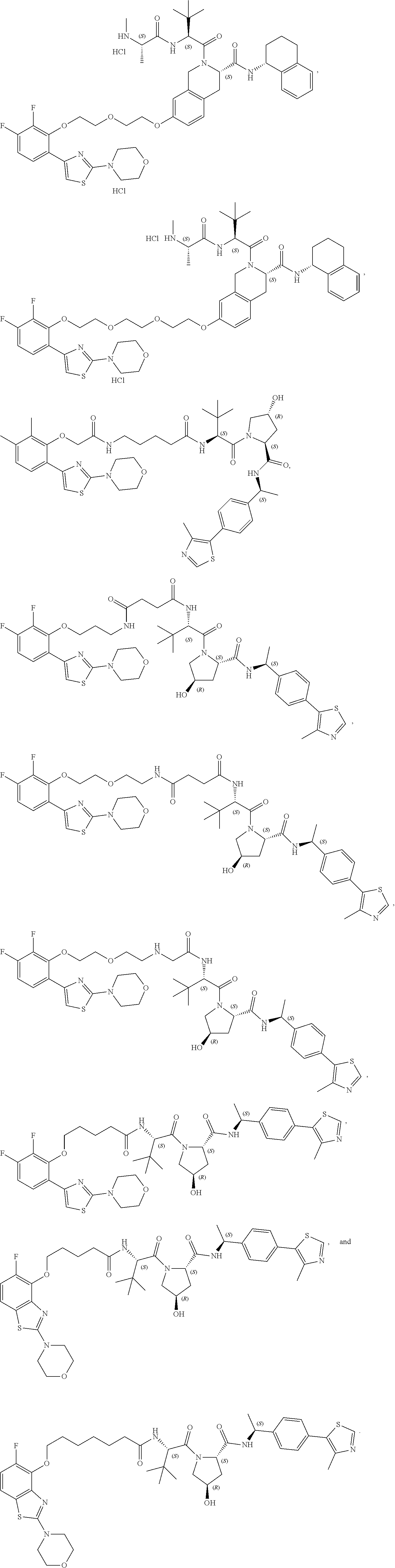

13. A pharmaceutical composition comprising a compound according to claim 1, and a pharmaceutically acceptable carrier, additive and/or excipient.

14. A method of treating a disease state or condition in a patient wherein dysregulated protein activity is responsible for said disease or condition, said method comprising administering an effective amount of a compound according to claim 1, to a patient in need thereof.

15. A method of degrading an androgen receptor in a cell, e.g., a mutated AR such as any AR-V1 to AR-V15 splice variant, e.g., the AR-V7 splice variant, said method comprising administering an effective amount of a compound according to claim 1, to such cell, e.g., a cancer cell.

16. A method of inducing apoptosis in a cell, e.g., a cancer cell, said method comprising administering an effective amount of a compound according to claim 1, to such cell.

17. (canceled)

18. (canceled)

19. A method of treating a disease state or condition in a patient wherein dysregulated protein activity is responsible for said disease or condition, said method comprising administering an effective amount of a compound according to claim 12, to a patient in need thereof.

20. A method of degrading an androgen receptor in a cell, e.g., a mutated AR such as any AR-V1 to AR-V15 splice variant, e.g., the AR-V7 splice variant, said method comprising administering an effective amount of a compound according to claim 12, to such cell, e.g., a cancer cell.

21. A method of inducing apoptosis in a cell, e.g., a cancer cell, said method comprising administering an effective amount of a compound according to claim 12, to such cell.
